SEQUENCE LISTING (38 sequences)

SEQ ID NO: 1 - protein, 31 aa, Homo sapiens
HAEGTFTSDVSSYLEGQAAKEFIAWLVKGRG

SEQ ID NO: 2 - protein, 180 aa, Homo sapiens
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPPRGPARFLPLPGLPPALPEPPGILAPQPPDVGSSDPLSMVGPSQGRSPSYAS

SEQ ID NO: 3 - protein, 31 aa, synthetic construct
  REGION 1..31: note = Synthetic Construct
  SITE 2: note = Xaa - Xaa is G
  SITE 16: note = Xaa - Xaa is E
  SITE 20: note = Xaa - Xaa is R or K or C
  SITE 21: note = Xaa - Xaa is C or E
  SITE 30: note = Xaa - Xaa is G or C
HXEGTFTSDVSSYLEXQAAXXFIAWLVKGXG

SEQ ID NO: 4 - protein, 31 aa, synthetic construct
  REGION 1..31: note = Synthetic Construct
HGEGTFTSDVSSYLEEQAAREFIAWLVKGRG

SEQ ID NO: 5 - protein, 31 aa, synthetic construct
  REGION 1..31: note = Synthetic Construct
HGEGTFTSDVSSYLEEQAACEFIAWLVKGGG

SEQ ID NO: 6 - protein, 31 aa, synthetic construct
  REGION 1..31: note = Synthetic Construct
HGEGTFTSDVSSYLEEQAARCFIAWLVKGGG

SEQ ID NO: 7 - protein, 31 aa, synthetic construct
  REGION 1..31: note = Synthetic Construct
HGEGTFTSDVSSYLEEQAAREFIAWLVKGCG

SEQ ID NO: 8 - protein, 31 aa, synthetic construct
  REGION 1..31: note = Synthetic Construct
HGEGTFTSDVSSYLEEQAAKEFIAWLVKGGG

SEQ ID NO: 9 - protein, 31 aa, synthetic construct
  REGION 1..31: note = Synthetic Construct
HGEGTFTSDVSSYLEEQAAREFIAWLVKGGG

SEQ ID NO: 10 - protein, 180 aa, synthetic construct
  REGION 1..180: note = Synthetic Construct
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPQNKSPHRDPPRGPARFLPLPGLPPALPEPPGILAPQPPDVGSSDPLSLVGPSQGRSPSYCS

SEQ ID NO: 11 - protein, 180 aa, synthetic construct
  REGION 1..180: note = Synthetic Construct
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPQNKSPHRDPPRGPARFLPLPGLPPALPEPPGILAPQPPDVGSSDPLSLVGGSQGRSPSYAS

SEQ ID NO: 12 - protein, 180 aa, synthetic construct
  REGION 1..180: note = Synthetic Construct
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPQNKSPHRDPPRGPARFLPLPGLPPALPEPPGILAPQPPDVGSSDPLSLVGGSQGRSPSYES

SEQ ID NO: 13 - protein, 17 aa, synthetic construct
  REGION 1..17: note = Synthetic Construct
GAQPGAQPGAQPGAQPA

SEQ ID NO: 14 - protein, 33 aa, synthetic construct
  REGION 1..33: note = Synthetic Construct
GAQPGAQPGAQPGAQPGAQPGAQPGAQPGAQPA

SEQ ID NO: 15 - protein, 65 aa, synthetic construct
  REGION 1..65: note = Synthetic Construct
GAQPGAQPGAQPGAQPGAQPGAQPGAQPGAQPGAQPGAQPGAQPGAQPGAQPGAQPGAQPGAQPA

SEQ ID NO: 16 - protein, 129 aa, synthetic construct
  REGION 1..129: note = Synthetic Construct
GAQPGAQPGAQPGAQPGAQPGAQPGAQPGAQPGAQPGAQPGAQPGAQPGAQPGAQPGAQPGAQPGAQPGAQPGAQPGAQPGAQPGAQPGAQPGAQPGAQPGAQPGAQPGAQPGAQPGAQPGAQPGAQPA

SEQ ID NO: 17 - protein, 18 aa, synthetic construct
  REGION 1..18: note = Synthetic Construct
GAQPGAQPCGAQPGAQPA

SEQ ID NO: 18 - protein, 14 aa, synthetic construct
  REGION 1..14: note = Synthetic Construct
GAQPGAQPCGAQPA

SEQ ID NO: 19 - protein, 123 aa, synthetic construct
  REGION 1..123: note = Synthetic Construct
HGEGTFTSDVSSYLEEQAAREFIAWLVKGRGGAQPGAQPGAQPGAQPAHPIPDSSPLLQFGGQVRQRYLYTDDAQQTEAHLEIREDGTVGGAADQSPESLLQLKALKPGVIQILGVKTSRFLC

SEQ ID NO: 20 - protein, 229 aa, synthetic construct
  REGION 1..229: note = Synthetic Construct
HGEGTFTSDVSSYLEEQAACEFIAWLVKGGGGAQPGAQPGAQPGAQPAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGQKSPHRDPAPRGPARFLPLPGLPPALPEPPGILAPQPPDVGSSDPLSLVGGSQGRSPSYAS

SEQ ID NO: 21 - protein, 229 aa, synthetic construct
  REGION 1..229: note = Synthetic Construct
HGEGTFTSDVSSYLEEQAAKCFIAWLVKGGGGAQPGAQPGAQPGAQPAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGQKSPHRDPAPRGPARFLPLPGLPPALPEPPGILAPQPPDVGSSDPLSLVGGSQGRSPSYAS

SEQ ID NO: 22 - protein, 229 aa, synthetic construct
  REGION 1..229: note = Synthetic Construct
HGEGTFTSDVSSYLEEQAAKCFIAWLVKGGGGAQPGAQPGAQPGAQPAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGQKSPHRDPAPRGPARFLPLPGLPPALPEPPGILAPQPPDVGSSDPLSLVGGSQGRSPSYES

SEQ ID NO: 23 - protein, 229 aa, synthetic construct
  REGION 1..229: note = Synthetic Construct
HGEGTFTSDVSSYLEEQAARCFIAWLVKGGGGAQPGAQPGAQPGAQPAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGQKSPHRDPAPRGPARFLPLPGLPPALPEPPGILAPQPPDVGSSDPLSLVGGSQGRSPSYES

SEQ ID NO: 24 - protein, 245 aa, synthetic construct
  REGION 1..245: note = Synthetic Construct
HGEGTFTSDVSSYLEEQAAKCFIAWLVKGGGGAQPGAQPGAQPGAQPGAQPGAQPGAQPGAQPAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGQKSPHRDPAPRGPARFLPLPGLPPALPEPPGILAPQPPDVGSSDPLSLVGGSQGRSPSYAS

SEQ ID NO: 25 - protein, 245 aa, synthetic construct
  REGION 1..245: note = Synthetic Construct
HGEGTFTSDVSSYLEEQAAKCFIAWLVKGGGGAQPGAQPGAQPGAQPGAQPGAQPGAQPGAQPAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGQKSPHRDPAPRGPARFLPLPGLPPALPEPPGILAPQPPDVGSSDPLSLVGGSQGRSPSYES

SEQ ID NO: 26 - protein, 277 aa, synthetic construct
  REGION 1..277: note = Synthetic Construct
HGEGTFTSDVSSYLEEQAAKCFIAWLVKGGGGAQPGAQPGAQPGAQPGAQPGAQPGAQPGAQPGAQPGAQPGAQPGAQPGAQPGAQPGAQPGAQPAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGQKSPHRDPAPRGPARFLPLPGLPPALPEPPGILAPQPPDVGSSDPLSLVGGSQGRSPSYES

SEQ ID NO: 27 - protein, 341 aa, synthetic construct
  REGION 1..341: note = Synthetic Construct
HGEGTFTSDVSSYLEEQAAKCFIAWLVKGGGGAQPGAQPGAQPGAQPGAQPGAQPGAQPGAQPGAQPGAQPGAQPGAQPGAQPGAQPGAQPGAQPGAQPGAQPGAQPGAQPGAQPGAQPGAQPGAQPGAQPGAQPGAQPGAQPGAQPGAQPGAQPGAQPAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGQKSPHRDPAPRGPARFLPLPGLPPALPEPPGILAPQPPDVGSSDPLSLVGGSQGRSPSYES

SEQ ID NO: 28 - protein, 229 aa, synthetic construct
  REGION 1..229: note = Synthetic Construct
HGEGTFTSDVSSYLEEQAAREFIAWLVKGCGGAQPGAQPGAQPGAQPAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGQKSPHRDPAPRGPARFLPLPGLPPALPEPPGILAPQPPDVGSSDPLSLVGGSQGRSPSYES

SEQ ID NO: 29 - protein, 230 aa, synthetic construct
  REGION 1..230: note = Synthetic Construct
HGEGTFTSDVSSYLEEQAAKEFIAWLVKGGGGAQPGAQPCGAQPGAQPAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGQKSPHRDPAPRGPARFLPLPGLPPALPEPPGILAPQPPDVGSSDPLSLVGGSQGRSPSYAS

SEQ ID NO: 30 - protein, 226 aa, synthetic construct
  REGION 1..226: note = Synthetic Construct
HGEGTFTSDVSSYLEEQAAREFIAWLVKGGGGAQPGAQPCGAQPAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGQKSPHRDPAPRGPARFLPLPGLPPALPEPPGILAPQPPDVGSSDPLSLVGGSQGRSPSYAS

SEQ ID NO: 31 - protein, 229 aa, synthetic construct
  REGION 1..229: note = Synthetic Construct
HGEGTFTSDVSSYLEEQAACEFIAWLVKGGGGAQPGAQPGAQPGAQPAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGQKSPHRDPAPRGPARFLPLPGLPPALPEPPGILAPQPPDVGSSDPLSLVGPSQGRSPSYCS

SEQ ID NO: 32 - protein, 229 aa, synthetic construct
  REGION 1..229: note = Synthetic Construct
HGEGTFTSDVSSYLEEQAAKCFIAWLVKGGGGAQPGAQPGAQPGAQPAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGQKSPHRDPAPRGPARFLPLPGLPPALPEPPGILAPQPPDVGSSDPLSLVGPSQGRSPSYCS

SEQ ID NO: 33 - protein, 229 aa, synthetic construct
  REGION 1..229: note = Synthetic Construct
HGEGTFTSDVSSYLEEQAARCFIAWLVKGGGGAQPGAQPGAQPGAQPAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGQKSPHRDPAPRGPARFLPLPGLPPALPEPPGILAPQPPDVGSSDPLSLVGPSQGRSPSYCS

SEQ ID NO: 34 - protein, 460 aa, synthetic construct
  REGION 1..460: note = Synthetic Construct
HGEGTFTSDVSSYLEEQAAREFIAWLVKGGGGAQPGAQPCGAQPGAQPAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGQKSPHRDPAPRGPARFLPLPGLPPALPEPPGILAPQPPDVGSSDPLSLVGPSQGRSPSYCSHGEGTFTSDVSSYLEEQAAREFIAWLVKGGGGAQPGAQPCGAQPGAQPAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGQKSPHRDPAPRGPARFLPLPGLPPALPEPPGILAPQPPDVGSSDPLSLVGPSQGRSPSYCS

SEQ ID NO: 35 - protein, 31 aa, synthetic construct
  REGION 1..31: note = Synthetic Construct
HGEGTFTSDVSSYLEEQAAKCFIAWLVKGGG

SEQ ID NO: 36 - protein, 182 aa, synthetic construct
  REGION 1..182: note = Synthetic Construct
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 37 - protein, 31 aa, synthetic construct
  REGION 1..31: note = Synthetic Construct
  SITE 2: note = Xaa - Xaa is Aib
  SITE 20: note = Lys - Lys is substituted
HXEGTFTSDVSSYLEGQAAKEFIAWLVRGRG

SEQ ID NO: 38 - protein, 182 aa, synthetic construct
  REGION 1..182: note = Synthetic Construct
  SITE 181: note = Cys - Cys is substituted
AHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGQKSPHRDPAPRGPARFLPLPGLPPALPEPPGILAPQPPDVGSSDPLSLVGPSQGRSPSYCS

(19) United States
(12) Patent Application Publication
(10) Pub. No.: US 2024/0400635 A1
(43) Pub. Date: Dec. 5, 2024

(54) FUSION COMPOUNDS AND USES THEREOF

(71) Applicant: Novo Nordisk A/S, Bagsvaerd (DK)

(72) Inventors: Birgitte Andersen, Maaloev (DK); Tina Moeller Tagmose, Ballerup (DK)

(21) Appl. No.: 18/778,518

(22) Filed: Jul. 19, 2024

Related U.S. Application Data
(63) Continuation of application No. PCT/EP2024/025136, filed on Mar. 27, 2024.

(30) Foreign Application Priority Data
Mar. 30, 2023 (EP) 23165485.6

(51) Int. Cl. C07K 14/605; A61K 38/00; A61P 3/04; C07K 14/50

(52) U.S. Cl. CPC C07K 14/605; A61P 3/04; C07K 14/50; A61K 38/00; C07K 2319/00

(57) ABSTRACT

The invention relates to fusion proteins comprising a GLP-1 polypeptide and an FGF21 polypeptide separated by spacers. The invention also relates to fusion compounds comprising the fusion proteins and at least one substituent. The invention also relates to the pharmaceutical use of the fusion compounds.

### TECHNICAL FIELD

The present invention relates to fusion compounds comprising a GLP-1 polypeptide and an FGF21 polypeptide. Furthermore, the invention relates to pharmaceutical compositions comprising such fusion compounds.

### CROSS-REFERENCE TO RELATED APPLICATIONS

This application is a Track 1 Continuation of International Application PCT/EP2024/025136, filed Mar. 27, 2024, which claims priority to European Patent Application 23165485.6, filed Mar. 30, 2023; the contents of which are incorporated herein by reference.

### INCORPORATION-BY-REFERENCE OF THE SEQUENCE LISTING

The instant application contains a Sequence Listing which has been submitted in XML format via the USPTO patent electronic filing system and is hereby incorporated by reference in its entirety. Said XML file, created on Jul. 15, 2024, is named 230018US01_Seq_List.xml and is 55 kilobytes in size.

### BACKGROUND

Fusion proteins can be generated by joining two or more genes by genetic engineering that originally coded for separate proteins. The result is a single polypeptide with functional properties of both parent proteins, i.e. a bifunctional fusion protein. The combination of unrelated proteins is challenging as it may prove difficult to manufacture due to incompatible properties. This can for instance cause aggregation or misfolding in one domain, while the conditions being perfect for the other domain. In addition, the two or more proteins (or polypeptide) that are fused together may have conflicting stability requirements which makes formulation challenging. Furthermore, it may be difficult to control and adjust the relative amounts of each component thus complicating dosing for optimal efficacy and safety. In addition, the different polypeptide moieties in a fusion protein may impact each other such that bioactivity is reduced, which is not desirable. Direct fusion of polypeptide moieties without a linker may lead to many undesirable outcomes, including misfolding of the fusion proteins, low yield in protein production, or impaired bioactivity.

GLP-1 is an incretin hormone produced by the endocrine cells of the intestine following ingestion of food. GLP-1 is a regulator of glucose metabolism, and the secretion of insulin from the beta cells of the islets of Langerhans in the pancreas. GLP-1 also causes insulin secretion in the diabetic state. Furthermore, via its ability to enhance satiety, GLP-1 reduces food intake, thereby limiting weight gain, and may even cause weight loss. Taken together, these actions give GLP-1 a unique profile, considered highly desirable for an antidiabetic agent, particularly since the glucose dependency of its antihyperglycemic effects should minimize any risk of severe hypoglycemia. However, its pharmacokinetic/pharmacodynamic profile is such that native GLP-1 is not therapeutically useful. GLP-1 is highly susceptible to enzymatic degradation in vivo, and cleavage by dipeptidyl peptidase IV (DPP-IV) is probably the most relevant, since this occurs rapidly and generates a noninsulinotropic metabolite. Thus, ways of prolonging the half-life of GLP-1 in vivo has attracted much attention. A range of different approaches have been used for modifying the structure of glucagon-like peptide 1 (GLP-1) compounds in order to provide a longer duration of action in vivo, which led to the development of once a week products like Albiglutide, Dulaglutide, and Semaglutide.

FGF21 belongs to the FGF19 subfamily of atypical fibroblast growth factors (FGFs) with metabolic rather than mitogenic effects. FGF21 binds and activates FGF receptors (FGFR1c, FGFR2c and FGFR3c) but only in the presence of the non-signaling co-receptor beta-klotho (BKL). Tissue specific expression of BKL determines the metabolic activity of FGF21. FGF21 transgenic mice are resistant towards diet-induced obesity and have increased longevity. FGF21 is a metabolic regulator of energy expenditure, glucose and lipid metabolism. FGF21 may have potential to reverse bodyweight, hyperglycaemia and dyslipidaemia in obese patients with diabetes and dyslipidaemia.

FGF21 suffers from in vivo instability due to proteolysis, and as much as half of the endogenous circulating human FGF21 is inactive. The loss of activity is due to degradation of the C-terminal, the majority of these metabolites terminate at P171 rather than S181. Protection against metabolic breakdown in the C-terminal region is therefore desirable for a therapeutic FGF21 molecule. Various approaches have been reported in attempting to increase the in vivo half-life of FGF21 recombinant proteins. One such as example is PEGylation. PEGylation in position 179 of [−1M, 179C]FGF21 results however in dramatic reduction in in vitro activity (J. Xu et al, Bioconjugate Chemistry (2013), 24, 915-925). Fc fusion technology has also been used. The Fc fusion protein resulting from attaching Fc to the C-terminus of FGF21 is however much less potent than native FGF21 and the N-terminal Fc fusion of FGF21 (Hecht et al, PLoS One 2012, 7(11), e49345). An Fc moiety has a molecular weight of about 50 kDa. Incorporation of Fc in a fusion protein thus increases its molecular weight by at least 50 kDa. An increase of molecular weight is however often not desirable as it makes the corresponding pharmaceutical formulation more challenging due to e.g. increased viscosity. In addition, the incorporation of an Fc domain may cause steric hindrance between the polypeptide moieties, which may lead to decreased bioactivity, and altered biodistribution and metabolism of the protein moieties due to the interference between domains.

Combined administration of GLP-1 and FGF21 has been reported (see WO 2010/142665). Co-administration of a FGF21 protein and a GLP-1 compound requires either injections of two separate products or a single injection of a co-formulation of two different compositions. Two injections would permit flexibility of dose amount and timing, but are inconvenient to patients both for compliance and pain. A co-formulation might also provide some flexibility of dose amounts, but it is often quite challenging or impossible to find formulation conditions that permit chemical and physical stability of both compositions due to different molecular characteristics of the two different products.

Hence, there is a need for fusion compounds comprising a GLP-1 polypeptide and an FGF21-polypeptide having improved bioavailability, extended half-life, and/or increased potency.

### SUMMARY

The invention relates to fusion compounds comprising a GLP-1 polypeptide and an FGF21 polypeptide, wherein the GLP-1 polypeptides and the FGF21 polypeptide are separated by a spacer.

In a first aspect, the fusion compound comprises a GLP-1 polypeptide, which is an analogue of SEQ ID NO:1 and a FGF21 polypeptide, which is an analogue of FGF21(1-181) SEQ ID NO:2, wherein the GLP-1 polypeptide and the FGF21 polypeptide are separated by a spacer comprising 1-257 amino acids. The fusion compound may comprise a substituent. In a second aspect, the invention relates to a pharmaceutical composition comprising a fusion protein or a fusion compound. In a third aspect, the invention relates to the use of the fusion protein or fusion compound for use in medicine. In a fourth aspect, the invention relates to the medical use of the fusion protein or fusion compound e.g. in the treatment of obesity and/or for improving lipid parameters and/or non-alcoholic fatty liver disease (NAFLD) such as non-alcoholic steatohepatitis (NASH). In a fifth aspect, the invention relates to a method of preparing the fusion protein or fusion compound.

### BRIEF DESCRIPTION OF DRAWINGS

FIG. 1 shows the cumulative food intake (g) after single intravenous dose of vehicle or active compound in lean mice.

FIG. 2 shows baseline corrected body weight data in LDLr−/− mice treated with Chem. 24, Chem. 25, Chem. 14, Chem. 16, Chem. 19, Chem. 12, and Chem. 23 for 21 days. Data: mean+/−SEM (n=9-10/group)

### DESCRIPTION

In what follows, “a” as used herein can mean “one or more” or “at least one”. Greek letters may be represented by their symbol or the corresponding written name, for example: α=alpha; β=beta; ε=epsilon; γ=gamma; ω=omega; etc. Also, the Greek letter of μ may be represented by “u”, e.g. in μl=ul, or in μM=uM.

An asterisk (*) in a chemical formula designates a point of attachment. In what follows, unless otherwise indicated in the specification, terms presented in singular form also include the plural situation, e.g. when referring to the “fusion compound”, it is to be understood that this embraces all individual variants falling within a broad definition of said fusion compound.

As used herein, the term “about” or “approximately”, when used together with a numeric value (e.g. 5, 10%, ⅓), refers to a range of numeric values that can be less or more than the number. For example, “about 5” refers to a range of numeric values that are 10%, 5%, 2%, or 1% less or more that 5, e.g. a range of 4.5 to 5.5, or 4.75 to 5.25, or 4.9 to 5.1, or 4.95 to 5.05. In some instances, “about 5” refers to a range of numeric values that are 2% or 1% less or more than 5, e.g. a range of 4.9 to 5.1 or 4.95 to 5.05. In some embodiments, the term “about” as used herein means±10% of the value referred to, and includes the value.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates to the contrary.

In a first aspect, the invention relates to a fusion protein comprising a polypeptide of formula Chem. 1: A-B-C, wherein:
A is a GLP-1 polypeptide, which is an analogue of SEQ ID NO:1;
B is a spacer consisting of 1-257 amino acids;
C is an FGF21 polypeptide, which is an analogue of SEQ ID NO: 2;
   or a pharmaceutically acceptable salt, amide, or ester thereof.

Also, or alternatively, the invention relates to a fusion compound comprising a polypeptide of formula Chem. 1: A-B-C, wherein:
A is a GLP-1 polypeptide, which is an analogue of SEQ ID NO:1;
B is a spacer consisting of 1-257 amino acids; and
C is an FGF21 polypeptide, which is an analogue of SEQ ID NO: 2;
   and a substituent; or a pharmaceutically acceptable salt, amide, or ester thereof.

Also, or alternatively, the invention relates to a fusion compound which is capable of activating the human GLP-1 receptor and/or is capable of activating the FGFR complex.

In a second aspect, the invention relates to a pharmaceutical composition comprising a fusion protein or fusion compound (i.e. a fusion protein or fusion compound as defined in the first aspect of the invention, including all embodiments and particular features thereof) and optionally one or more pharmaceutically acceptable excipients.

In a third aspect, the invention relates to a fusion protein or a fusion compound (i.e. a fusion protein or fusion compound as defined in the first aspect of the invention, including all embodiments and particular features thereof) or a pharmaceutical composition (i.e. a composition as defined in the third aspect of the invention, including all embodiments and particular features thereof), for use as a pharmaceutical or for use in medicine.

In a fourth aspect, the invention relates to a fusion protein or a fusion compound (i.e. a fusion protein or fusion compound as defined in the first aspect of the invention, including all embodiments and particular features thereof) or a pharmaceutical composition (i.e. a composition as defined in the third aspect of the invention, including all embodiments and particular features thereof), for use in (i) prevention and/or treatment of all forms of diabetes; (ii) delaying or preventing diabetic disease progression, and/or delaying the progression of non-insulin requiring type 2 diabetes to insulin requiring type 2 diabetes; (iii) improving β-cell function; (iv) prevention and/or treatment of cognitive disorders and/or neurodegenerative disorders; (v) prevention and/or treatment of eating disorders; and/or prevention and/or treatment of comorbidities to obesity; (vi) prevention and/or treatment of diabetic complications; (vii) improving lipid parameters; (viii) prevention and/or treatment of cardiovascular diseases; and/or reduction of blood pressure; (ix) prevention and/or treatment of gastrointestinal diseases; (x) prevention and/or treatment of critical illness; prevention or reduction of the likelihood of a patient suffering from bacteraemia, septicaemia, and/or septic shock during hospitalisation; and/or stabilising blood glucose, insulin balance and optionally metabolism in intensive care unit patients with acute illness; (xi) prevention and/or treatment of polycystic ovary syndrome (PCOS); (xii) prevention and/or treatment of cerebral disease, such as cerebral ischemia, cerebral haemorrhage, and/or traumatic brain injury; (xiii) prevention and/or treatment of sleep apnoea; (xiv) prevention and/or treatment of abuse, such as alcohol abuse and/or drug abuse; (xv) prevention and/or treatment of dyslipidemia; (xv) treatment and/or prevention of hepatic steatosis and/or (xvi) non-alcoholic fatty liver disease (NAFLD) and/or acute and chronic pancreatitis.

In a fifth aspect, the invention relates to a method of preparing fusion proteins or fusion compounds (i.e. a fusion protein or fusion compound as defined in the first aspect of the invention, including all embodiments and particular features thereof).

### General Definitions

The term “compound” as used herein refers to a molecular entity, and “compounds” may thus have different structural elements besides the minimum element defined for each compound or group of compounds. The fusion compound may be referred to as “compound”, and the term “compound” is also meant to cover pharmaceutically relevant forms hereof, i.e. the invention relates to a compound as defined herein or a pharmaceutically acceptable salt, amide, or ester thereof.

The term “polypeptide” or “polypeptide sequence”, as used herein refers to a compound which comprises a series of amino acids interconnected via amide (or peptide) bonds. The term polypeptide is used interchangeably with the term “peptide” and the term “protein”.

The term “moiety” relates to a fragment or part of a molecule such as a fusion compound or a fusion protein.

The term “polypeptide moiety” relates to a polypeptide fragment or part of a molecules such as fusion compound or fusion protein. For example, “GLP-1 polypeptide moiety” refers to A in Chem. 1 while “FGF21 polypeptide moiety refers to C in Chem. 1. For example, the term “spacer moiety” refers to B in Chem. 1. Put differently, the term “GLP-1 polypeptide moiety” relates to the “GLP-1 polypeptide” fragment or part of Chem. 1.

The term “analogue” as used herein generally refers to a polypeptide, the sequence of which has one or more amino acid changes as compared to a reference amino acid sequence. Said amino acid changes may include amino acid additions, amino acid deletions, and/or amino acid substitutions. Amino acid “substitutions” may also be referred to as “mutations”. In particular embodiments, an analogue “comprises” specified changes. In other particular embodiments, an analogue “consists of” or “has” specified changes. When the term “comprises” or “comprising” is used in relation to amino acid changes in an analogue, it should be understood that the analogue may have further amino acid changes as compared to its reference sequence. When the term “consisting of” or “has” is used in relation to amino acid changes in an analogue, it should be understood that the specified amino acid mutations are the only amino acid changes in the analogue as compared to the reference sequence. In the context of this application, the term “analogue” designates analogues of human glucagon-like peptide-1 GLP-1(7-37) (SEQ ID NO: 1), analogues of human endogenous FGF21 (FGF21(1-181) (SEQ ID NO: 2) and/or analogues of fusion compounds.

The term “derivative” generally refers to a polypeptide which may be prepared from a native polypeptide or an analogue thereof by chemical modification, in particular by covalent attachment of one or more substituents. A derivative can also be referred to as an alkylated analogue. For example, fusion compounds as defined herein are derivatives of fusion proteins as derived herein.

The term “amino acid” as used herein refers to any amino acid, i.e. both proteinogenic amino acids and non-proteinogenic amino acids. The term “proteinogenic amino acids” as used herein refers to the 20 standard amino acids encoded by the genetic code in humans. The term “non-proteinogenic amino acids” as used herein refers to all amino acids which don't qualify as proteinogenic amino acids. In general, amino acid residues, e.g. in context of a polypeptide sequence, as used herein, may be identified by their full name, their one-letter code, and/or their three-letter code. These three ways are fully equivalent and interchangeable. In what follows, each amino acid of the peptides of the invention for which the optical isomer is not stated is to be understood to mean the L-isomer (unless otherwise specified).

The terms “fusion” and “fused” as used herein refers to a compound comprising two or more individually defined polypeptides which are covalently linked by a peptide bond or by a spacer. The term “spacer” as used herein refers to a molecular moiety which separates two individually defined polypeptides.

The term “bifunctional” means bifunctionally active. For example, in a bifunctional fusion compound both polypeptides of the bifunctional fusion protein—such as GLP-1 and FGF21—are active.

The term “sequence identity” as used herein refers to the extent to which two amino acid sequences (e.g. polypeptides) have the same residues at the same positions in an alignment. This may also be referred to merely as “identity”. The sequence identity is conveniently expressed as a percentage, i.e. if 85 amino acids out of 100 aligned positions between the two sequences are identical the degree of identity is 85%. For purposes of the present invention, the sequence identity between two amino acid sequences is determined by using simple handwriting and eyeballing; and/or a standard protein or peptide alignment program, such as “align” which is based on a Needleman-Wunsch algorithm. This algorithm is described in Needleman, S. B. and Wunsch, C. D., (1970), Journal of Molecular Biology, 48: 443-453, and the align program by Myers and W. Miller in “Optimal Alignments in Linear Space” CABIOS (computer applications in the biosciences) (1988) 4:11-17. For the alignment, the default scoring matrix BLOSUM62 and the default identity matrix may be used, and the penalty for the first residue in a gap may be set at −12, or preferably at −10, and the penalties for additional residues in a gap at −2, or preferably at −0.

The term “FGFR complex” “as used herein refers to the FGF receptor β-klotho (FGFR-BKL) complex such as FGFR1c, such as FGFR3, such as FGFR2.

### GLP-1 Polypeptide

The term “GLP-1 polypeptide” as used herein refers to an analogue (or variant) of the human glucagon-like peptide-1 (GLP-1(7-37)), the sequence of which is included in the sequence listing as SEQ ID NO: 1 HAEGTFTSDVSSYLEGQAAKEFIAWLVKGRG. The polypeptide of SEQ ID NO: 1 may also be designated “native human GLP-1”. The GLP-1 polypeptide has one or more amino acid changes as compared to native human GLP-1, wherein said amino acid changes may be in the form of amino acid additions, amino acid deletions, and/or amino acid substitutions. The numbering of amino acid residues in the GLP-1 polypeptides of the fusion compounds or fusion proteins of the invention follows the established practice in the art for native human GLP-1, namely that the first (N-terminal) amino acid residue is numbered or accorded position no. 7, and the subsequent amino acid residues downstream towards the C-terminus are numbered 8, 9, 10, and so on, until the last (C-terminal) amino acid residue. In native human GLP-1 the C-terminal amino acid residue is Gly, with number 37. The numbering is done differently in the sequence listing, where the first amino acid residue of SEQ ID NO: 1 (His) is assigned no. 1, and the last (Gly) no. 31. However, herein we follow the established numbering practice in the art, as explained above, i.e. the first (N-terminal) amino acid residue is given the number 7 in SEQ ID 1. GLP-1 polypeptides of the fusion compounds or fusion proteins of the invention, i.e. the GLP-1 polypeptide moiety of the fusion compound or fusion protein, may be described by reference to i) the nature of the actual change, and to ii) the position of the amino acid residue in native which is changed. Thus, an amino acid change in the form of a substitution may be referred to as “Xaa”, wherein aa is the amino acid introduced in the substituted position, and wherein the X is a number which corresponds to the position of the amino acid residue of SEQ ID NO: 1 which is substituted.

The GLP-1 polypeptide moiety of a fusion compound or fusion protein of the invention may e.g. be referred to with reference to amino acid changes relative to that of GLP-1(7-37) (SEQ ID NO: 1), e.g. as the following: ‘[8G, 22E, 27C, 36G]GLP-1(7-37)’. In this example the GLP-1 polypeptide is an analogue of GLP-1(7-37) (SEQ ID NO: 1), wherein the analogue has Gly in a position corresponding to position 8 of GLP-1(7-37), has Glu in a position corresponding to position 22 of GLP-1(7-37), has Cys in a position corresponding to position 27 of GLP-1(7-37), and has Gly in a position corresponding to position 36 of GLP-1(1-37) (SEQ ID NO: 1). If amino acid changes in a GLP-1 polypeptide are presented as relative to GLP-1(7-37) (SEQ ID NO: 1), it is understood that it refers to changes to the GLP-1 polypeptide moiety alone and does not involve any other moiety in the fusion compound or fusion protein such as the spacer or the FGF21 polypeptide moiety.

“GLP-1 polypeptide” is used interchangeably with “GLP-1 analogue” and “GLP-1 variant”. A GLP-1 polypeptide “comprising” certain specified changes may comprise further changes, when compared to GLP-1(7-37) (SEQ ID NO:1).

In some embodiments, the GLP-1 polypeptide moiety of the fusion protein or fusion compound is an analogue of SEQ ID NO:1. In some embodiments, the GLP-1 polypeptide moiety is at least 50%, preferably at least 55%, preferably at least 60%, preferably at least 65%, preferably at least 70%, preferably at least 75%, preferably at least 80%, preferably at least 85%, preferably at least 90% identical to SEQ ID NO: 1. In some embodiments, the GLP-1 polypeptide moiety has a maximum of 12 amino acid changes, preferably a maximum of 11 amino acid changes, preferably a maximum of 10 amino acid changes, preferably a maximum of 9 amino acid changes, preferably a maximum of 8 amino acid changes, preferably a maximum of 7 amino acid changes, preferably a maximum of 6 amino acid changes, preferably a maximum of 5 amino acid changes, preferably a maximum of 5 amino acid changes, preferably a maximum of 4 amino acid or preferably a maximum of 3 amino acid changes, as compared to SEQ ID NO: 1. In some embodiment, the GLP-1 polypeptide moiety has 3-7 changes as compared to SEQ ID NO: 1.

In some embodiments, the GLP-1 polypeptide comprises or consists of the amino acid sequence H-Xaa₈-E-G-T-F-T-S-D-V-S-S-Y-L-E-Xaa₂₂-Q-A-A-Xaa₂₆-Xaa₂₇-F—I-A-W-L-V-K-G-Xaa₃₆-G (SEQ ID NO: 3), wherein Xaa₈ is G; Xaa₂₂ is E; Xaa₂₆ is R, C or K; Xaa₂₇ is C or E; Xaa₃₆ is G or C.

In some embodiments, the GLP-1 polypeptide is capable of undergoing Cys-alkylation.

Non-limiting examples of GLP-1 polypeptides are provided in Table 1.

TABLE 1
Non-limiting examples of GLP-1 polypeptide moieties of
fusion proteins or fusion compounds of the invention.

| GLP-1 polypeptides | SEQ ID NO |
|---|---|
| [8G, 22E, 26R]GLP-1(7-37) | SEQ ID NO: 4 |
| [8G, 22E, 26C, 36G]GLP-1(7-37) | SEQ ID NO: 5 |
| [8G, 22E, 26R, 27C, 36G]GLP-1(7-37) | SEQ ID NO: 6 |
| [8G, 22E, 26R, 36C]GLP-1(7-37) | SEQ ID NO: 7 |
| [8G, 22E, 36G]GLP-1(7-37) | SEQ ID NO: 8 |
| [8G, 22E, 26R, 36G]GLP-1(7-37) | SEQ ID NO: 9 |
| [8G, 22E, 27C, 36G]GLP-1(7-37) | SEQ ID NO: 35 |

### FGF21 Polypeptide

The terms “human FGF21”, “native FGF21”, “wildtype FGF21”, “human endogenous FGF21”, and “FGF21(1-181)” are used interchangeably and refer to a polypeptide consisting of sequence SEQ ID NO: 2: HPIPDSSPLLQFGGQVRQRYLYTDDAQQTEAHLEIREDGTVGGAADQSPESLLQLKALKP GVIQILGVKTSRFLCQRPDGALYGSLHFDPEACSFRELLLEDGYNVYQSEAHGLPLHLPGNK SPHRDPPRGPARFLPLPGLPPALPEPPGILAPQPPDVGSSDPLSMVGPSQGRSPSYAS. In this formula the numbering of the amino acid residues follows the numbering for FGF21(1-181) (SEQ ID NO: 2), wherein the first (N-terminal) amino acid residue (H) is numbered according to position no. 1 and the subsequent amino acid residues towards the C-terminus are numbered 2, 3, 4 and so on, until the last (C-terminal) amino acid residue (S), which in FGF21(1-181) is position no. 181. In the sequence listing the first amino acid residue of SEQ ID NO: 2 (H) is assigned no. 1, and the last (S) no. 181, and thus the numbering herein and in the sequence listing is identical. The same applies for other FGF21 polypeptide sequences.

The term “FGF21 polypeptide” is used interchangeably with “FGF21 analogue” and “FGF21 variant”.

The term “FGF21 polypeptide” as used herein refers to a polypeptide which is capable of activating human FGF21 receptors. In some embodiments, the FGF21 polypeptide moiety of the fusion compound or fusion protein is capable of activating FGF21 receptors. The term “FGF21 analogue” as used herein refers to a polypeptide which is an analogue of FGF21(1-181). In some embodiments, the FGF21 polypeptide moiety of the fusion compound is an analogue of FGF21(1-181). In some embodiment, the FGF21 polypeptide moiety of the fusion compound is an analogue of SEQ ID NO: 2. ‘[121Q, 168L, 180C]FGF21(1-181)’. In this example the FGF21 polypeptide is an analogue of FGF21(1-181), wherein the analogue has Gln in a position corresponding to position 121 of FGF21(1-181), has Leu in a position corresponding to position 168 of FGF21(1-181), and has Cys in a position corresponding to position 180 of FGF21(1-181). The addition of Ala at the N-terminus may also be referred to as “−1A” or “−1Ala”, since it corresponds to position −1 of the FGF21(1-181). If amino acid changes in an FGF21 polypeptide is presented as relative to FGF21(1-181), it is understood that it refers to changes to the FGF21 polypeptide moiety alone and does not involve any other moiety in the fusion compound such as the spacer or the GLP-1 polypeptide moiety.

If amino acid changes in an FGF21 polypeptide is presented as relative to FGF21(1-181) (SEQ ID NO:2), it is understood that it refers to changes to the FGF21 polypeptide moiety alone and does not involve any other moiety in the fusion compound such as the spacer or the GLP-1 polypeptide moiety.

In some embodiments, the FGF21 polypeptide moiety of the fusion compound or fusion protein is an analogue of SEQ ID NO: 2. In some embodiments, the FGF21 polypeptide is at least 50%, preferably at least 55%, preferably at least 60%, preferably at least 65%, preferably at least 70%, preferably at least 75%, preferably at least 80%, preferably at least 85%, preferably at least 90%, or most preferably at least 95%, identical to SEQ ID NO: 2. In some embodiments, the FGF21 polypeptide comprises a maximum of 15 amino acid changes, preferably a maximum of 14 amino acid changes, preferably a maximum of 13 amino acid changes, preferably a maximum of 12 amino acid changes, preferably a maximum of 11 amino acid changes, preferably a maximum of 10 amino acid changes, preferably a maximum of 9 amino acid changes, preferably a maximum of 8 amino acid changes, preferably a maximum of 7 amino acid changes, preferably a maximum of 6 amino acid changes, preferably a maximum of 5 amino acid changes, preferably a maximum of 5 amino acid changes, preferably a maximum of 4 amino acid changes, or preferably a maximum of 3 amino acid changes, as compared to SEQ ID NO: 2. In some embodiments, the FGF21 polypeptide has 4 amino acid changes, as compared to SEQ ID NO: 2. In some embodiment, the FGF21 polypeptide comprises 180C. In some embodiments, the FGF21 polypeptide comprises or has the following amino acid changes as compared to SEQ ID NO: 2: [121Q, 168L, 180C] or [121Q, 168L, 171G], or [121Q, 168L, 171G, 180E]. In some embodiments, the FGF21 polypeptide moiety of the fusion compound is selected from a list consisting of SEQ ID NO: 10, SEQ ID NO: 11, and SEQ ID NO: 12. In some embodiments, the FGF21 polypeptide has FGF21 activity. In some embodiments, the FGF21 is capable of activating the FGFR1c. In some embodiments, the FGF21 is capable of activating the FGFR3c.

In some embodiments, the FGF21 polypeptide is capable of undergoing Cys-alkylation.

Non-limiting examples of FGF21 polypeptides are provided in Table 2.

TABLE 2
Non-limiting examples of FGF21 polypeptide moieties of the
fusion proteins or fusion compounds of the invention.

| FGF21 polypeptides | SEQ ID NO |
|---|---|
| [121Q, 168L, 180C]FGF21(1-181) | SEQ ID NO: 10 |
| [121Q, 168L, 171G]FGF21(1-181) | SEQ ID NO: 11 |
| [121Q, 168L, 171G, 180E]FGF21(1-181) | SEQ ID NO: 12 |

### Spacer

Frequently, fusion compounds or fusion proteins include a spacer that separates the bioactive moieties of the compound to ensure that any functionality residing in the bioactive moieties is not disturbed by the proximity of the other bioactive moiety.

The term “spacer”, as used herein, refers to an element that covalently connects the bioactive moieties of the fusion compounds or fusion proteins of the invention. The spacer may also be referred to as “spacer polypeptide”, “spacer moiety” or “spacer element”. In some embodiments, the spacer comprises an amino acid sequence whose N-terminus is connected to the C-terminus of the GLP-1 polypeptide moiety via an amide bond, and whose C-terminus is connected to the N-terminus of the FGF21 polypeptide moiety via an amide bond. In some embodiments, the spacer comprises repetitive elements of the formula Chem. 2 (GAQP)ₓ—C_{y}-(GAQP)ₕ-Aⱼ, wherein x is an integer in the range of 0-64, y is an integer in the range of 0-1, h is an integer in the range of 1-64 and j is an integer in the range of 0-1. An example of spacer nomenclature as used herein is: ‘[GAQP]₈-A’ or ‘(GAQP)x8, A’ which both are a short name for a polypeptide sequence consisting of 8 segments of GAQP followed by one segment of A. The sequence written in full GAQPGAQPGAQPGAQPGAQPGAQPGAQPGAQPA.

The spacer may impact the pharmacokinetic properties of the fusion compounds, e.g. by increasing the half-life of the fusion compound. In some embodiments, the spacer may be capable of improving the half-life of the fusion compound. The expression “improving half-life” of a fusion compound may mean that it extends the plasma half-life of a fusion compound such that it is suitable for a once daily injection or a twice-weekly injection, preferably for a once weekly injection.

In some embodiments, the spacer may consist of 1-257 amino acids, such as 5 to 257, such as 9 to 129. In some embodiments, the spacer may comprise at least two segments of GAQP. In some embodiments, the spacer may comprise (GAQP)₃₂-A. In some embodiments, the spacer is selected from a list consisting of: SEQ ID NO: 13, SEQ ID NO: 14, SEQ ID NO: 15, SEQ ID NO: 16, SEQ ID NO: 17, and SEQ ID NO: 18. In some embodiments, the spacer comprises or consists of SEQ ID NO: 13, SEQ ID NO:14, SEQ ID NO: 15, SEQ ID NO: 16, SEQ ID NO: 17 or SEQ ID NO: 18.

In some embodiments, the spacer is capable of undergoing Cys-alkylation.

Non-limiting examples of spacers are provided in Table 3.

TABLE 3
Non-limiting examples of spacers.

| Spacer | SEQ ID NO |
|---|---|
| 4 × GAQP-A | SEQ ID NO: 13 |
| 8 × GAQP-A | SEQ ID NO: 14 |
| 16 × GAQP-A | SEQ ID NO: 15 |
| 32 × GAQP-A | SEQ ID NO: 16 |
| 2 × GAQP-C-2 × GAQP-A | SEQ ID NO: 17 |
| 2 × GAQP-C-GAQPA | SEQ ID NO: 18 |

### Substituent

The term “substituent”, as used herein, refers to a moiety that is covalently attached to the GLP-1 polypeptide or FGF21 polypeptide or spacer of the compounds of the invention. The term “substituent” and “side chain” are used interchangeably. The substituent is capable of forming non-covalent binding with albumin, thereby promoting the circulation of the derivative in the blood stream, and also having the effect of protracting the time of action of the fusion compound, due to the fact that the association of the fusion compound to albumin is only slowly disassociated to release the free form of the derivative. Thus, the substituent as a whole, may also be referred to as an “albumin-binding moiety”. The substituent comprises a portion which is particularly relevant for the albumin binding and thereby the protraction, which portion may be referred to as a “protracting moiety” or “protractor”. The protracting moiety may be near, preferably at, the terminal (or distal, or free) end of the substituent, relative to its point of attachment to the peptide. The substituent may comprise a portion between the protracting moiety and the point of attachment to the peptide, which portion may be referred to as a “linker”. The “substituent”, may be lipophilic, and/or negatively charged at physiological pH (7.4). The “protractor” or “substituent”, may be covalently attached to the thiol group of a cysteine residue of the GLP-1 polypeptide, the FGF21 analogue or the spacer by alkylation. The substituent may be synthesised as and activated with a haloacetamide group, capable of reacting with the thiol group of a cysteine residue, under formation of a covalent thiol-carbon bond (this process being referred to as Cys-alkylation) which is also referred to as a thio-ether bond. The halogen atom is thus not present in the derivatives, and the substituent is linked through the sulfur atom. In cases where the thiol group is mentioned in relation to a derivative it must be understood as the sulfur atom which is part of the thiol group of the cysteine prior to Cys-alkylation. Alternatively, the substituent may be activated with a maleimide group, which reacts with the thiol group of a cysteine residue, under formation of a covalent thiol-carbon bond. The substituent may function as an albumin binder or albumin binding moiety. The substituent may be an albumin binder or albumin binding moiety.

Protractor: The protractor may be at, or near, the distant end of the side chain, relative to its point of attachment to the protein. In one aspect, each protractor comprises, or consists of, a protractor of formula Chem. 3: HOOC—(CH₂)x-CO—*. The length of the carbon chain defined by x may vary from 8-18, such as 14-18 or such as 14-16.

The nomenclature is as is usual in the art, for example in the above formulas *—CO—* refers to carbonyl (*—C(═O)—*). For example, in any formula (R—CO—*) herein (where R is as defined by each formula), R—CO—* refers to R—C(═O)—*.

Linker: The linker may comprise at least one of the following linker elements Chem. 4, Chem. 5, and Chem. 6. The elements Chem. 4 and Chem. 5 each individually hold a —NH— and CO-end allowing them to be linked by amide bonds to each other and to either —CO— or —NH— of Chem. 3 or Chem. 6.

Chem. 6 has a —NH— end (capable of forming an amide bond with either Chem. 3 or Chem. 4 or Chem. 5), and a —NH—CO—CH₂— end, which in its unreacted form is a haloacetamide capable of reacting with the thiol group of a cysteine incorporated in the GLP-1 polypeptide moiety, a cysteine incorporated in the spacer moiety or a cysteine incorporated in the FGF21 polypeptide moiety of the fusion protein or the fusion compound of the invention.

The linker elements Chem. 4, Chem. 5, and Chem. 6, are as follows:

Chem. 4 is *—NH—CH(COOH)—(CH₂)₂—CO—*;

Chem. 5 is *—NH—(CH₂)₂—[O—(CH₂)₂]ₖ—O—[CH₂]ₘ—CO—*, wherein

k is an integer in the range of 1 to 5, wherein m is an integer in the range of 1 to 5;

Chem. 6 is *—NH—(CH₂)n-NH—CO—CH₂—*, wherein n is an integer in the range of 1 to 5.

Chem. 4, Chem. 5, and Chem. 6 may be interconnected via amide bonds, connected at their *—NH end to the CO—* end of the protractor according to Chem. 3, and at their CH₂—* end to an amino acid such as cysteine of A, B or C.

Chem 4. may also be referred to herein as gGlu or gamma Glu or γGlu.

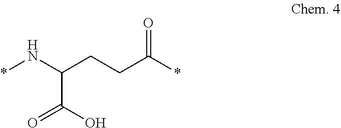 [SMILES: *NC(CCC(*)=O)C(=O)O]

Chem. 5 may also be referred to as “Ado” herein.

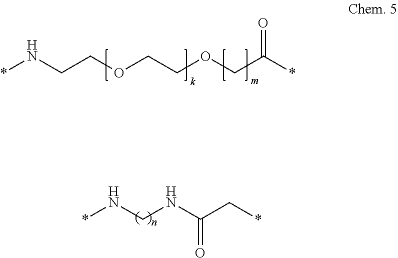

In some embodiments, the substituent is Chem. 7:

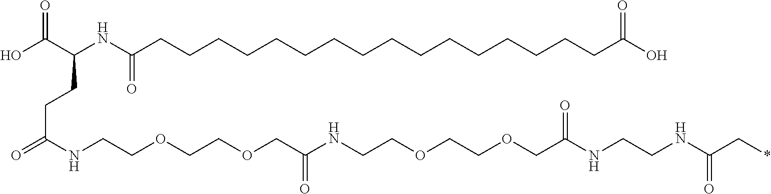 [SMILES: *CC(=O)NCCNC(=O)COCCOCCNC(=O)COCCOCCNC(=O)CC[C@H](NC(=O)CCCCCCCCCCCCCCCCC(=O)O)C(=O)O]

In one embodiment, the substituent is [2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl] (Chem. 7).

In some embodiments, a fusion compound comprises one or more substituents such as a first and a second substituent. In some embodiments, a fusion compound comprises at least one substituent, such as one, such as two, such as three, such as seven substituents. The substituent may be attached to any amino acid residue in a fusion compounds. In some embodiments, the substituent is attached to a Cys residue incorporated in a fusion compound. In some embodiments, the substituent is attached to a Cys residue of the GLP-1 polypeptide moiety of the fusion compound. In some embodiments, the substituent is attached to a Cys residue in the FGF21 polypeptide moiety of a fusion compound. In some embodiments, the substituent is attached to a Cys residue in the spacer moiety of a fusion compound. In some embodiments, a first substituent is attached to a Cys residue of the GLP-1 polypeptide moiety and a second substituent is attached to a Cys residue of the FGF21 polypeptide moiety of a fusion compound. In some embodiments, a first substituent is attached to a Cys in the spacer and a second substituent is attached to a Cys in the FGF21 polypeptide. In some embodiments, the substituent is attached to an amino acid residue of the polypeptide backbone of a fusion compound. In some embodiment, the substituent is attached to the spacer moiety or to the FGF21 polypeptide moiety of a fusion compound. In some embodiments, the substituent is attached to a Cys residue of the GLP-1 polypeptide moiety of a fusion compound, wherein said Cys residue is in position 27 or 26. In some embodiments, the substituent is attached to a Cys residue of the FGF21 polypeptide moiety of a fusion compound, wherein said Cys residue is in position 180.

### Fusion Protein

Fusion proteins, are proteins created through the joining of two or more genes which originally coded for separate proteins. Translation of this fusion gene results in a single polypeptide with functional properties derived from each of the original proteins. Fusion proteins may comprise two or more bioactive moieties, which exert their bioactivity mainly via interaction with two distinct sites. A GLP-1/FGF21 fusion protein therefore comprises a GLP-1 polypeptide and a FGF21 polypeptide. The GLP-1/FGF21 fusion protein may further comprise a peptide spacer. A GLP-1/FGF21 fusion protein therefore comprises a GLP-1 polypeptide moiety, a FGF21 polypeptide moiety and optionally a spacer moiety. Fusion proteins comprising a GLP-1 polypeptide and a FGF21 polypeptide may be fused such that the C-terminus of the GLP-1 polypeptide is fused to the N-terminus of a peptide spacer, and the C-terminus of the peptide spacer is fused to the N-terminus of the FGF21 polypeptide. The GLP-1 peptide exerts its bioactivity via the GLP-1R, and the latter exerts its bioactivity mainly via activation of an FGFR complex.

The complete linear string of amino acids forming the bioactive polypeptides (e.g. a GLP-1 polypeptide and an FGF21 polypeptide) and any spacer separating the bioactive polypeptides, may be referred to herein as the “polypeptide backbone”. The term “fusion protein” and “polypeptide backbone” is used interchangeably herein. As such the polypeptide backbone of the fusion protein does not include the substituent. If a substituent is present the compound is referred to herein as “fusion compound” instead of “fusion protein”. The difference between a fusion protein as defined herein and a fusion compound as defined herein is that the fusion compound may comprise one or more substituents. The fusion proteins of the invention may be incorporated in the fusion compounds of the invention.

In some embodiments, the fusion protein is a bifunctional fusion protein. In some embodiment, the fusion protein is a fusion protein according to A-B-C(Chem. 1), wherein A is a GLP-1 polypeptide, which is an analogue of SEQ ID NO: 1; B is a peptide spacer consisting 1-257 amino acids; and C is a FGF21 analogue, which is an analogue of SEQ ID NO: 2. In some embodiment, A is a GLP-1 polypeptide comprising or consisting of the amino acid sequence H-Xaa₈-E-G-T-F-T-S-D-V-S-S-Y-L-E-Xaa₂₂-Q-A-A-Xaa₂₆-Xaa₂₇-F—I-A-W-L-V-K-G-Xaa₃₆-G (SEQ ID NO: 3), wherein Xaa₈ is G; Xaa₂₂ is E; Xaa₂₆ is R, C or K; Xaa₂₇ is C or E; Xaa₃₆ is G or C. In some embodiments, B is a peptide spacer, wherein the peptide spacer is 5 to 257 amino acids in length. In some embodiments, the C-terminus of the GLP-1 polypeptide is fused to the N-terminus of the peptide spacer, and the C-terminus of the peptide spacer is fused to the N-terminus of the FGF21 analogue. In some embodiments, the fusion protein is selected from a list consisting of SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, and SEQ ID NO: 34. In some embodiments, the fusion protein is selected from a list consisting of SEQ ID NO: 26, SEQ ID NO: 27, SEQ ID NO: 32, and SEQ ID NO: 33. In some embodiments, the fusion protein is SEQ ID NO: 27.

In some embodiments, the fusion protein according to A-B-C(Chem. 1) is incorporated in the fusion compounds of the inventions.

Non-limiting examples of fusion proteins are provided in Table 4.

TABLE 4
Non-limiting examples of fusion proteins capable
of being incorporated in fusion compounds of
the invention.

| Fusion protein backbone | SEQ ID NO |
|---|---|
| HGEGTFTSDVSSYLEEQAAREFIAWLVKGRGGAQPGAQPGAQPGAQ | SEQ ID NO: 19 |
| PAHPIPDSSPLLQFGGQVRQRYLYTDDAQQTEAHLEIREDGTVGGAA | |
| DQSPESLLQLKALKPGVIQILGVKTSRFLCQRPDGALYGSLHFDPEACS | |
| FRELLLEDGYNVYQSEAHGLPLHLPGQKSPHRDPAPRGPARFLPLPG | |
| LPPALPEPPGILAPQPPDVGSSDPLSLVGPSQGRSPSYCS | |
| HGEGTFTSDVSSYLEEQAACEFIAWLVKGGGGAQPGAQPGAQPGAQ | SEQ ID NO: 20 |
| PAHPIPDSSPLLQFGGQVRQRYLYTDDAQQTEAHLEIREDGTVGGAA | |
| DQSPESLLQLKALKPGVIQILGVKTSRFLCQRPDGALYGSLHFDPEACS | |
| FRELLLEDGYNVYQSEAHGLPLHLPGQKSPHRDPAPRGPARFLPLPG | |
| LPPALPEPPGILAPQPPDVGSSDPLSLVGGSQGRSPSYAS | |
| HGEGTFTSDVSSYLEEQAAKCFIAWLVKGGGGAQPGAQPGAQPGAQ | SEQ ID NO: 21 |
| PAHPIPDSSPLLQFGGQVRQRYLYTDDAQQTEAHLEIREDGTVGGAA | |
| DQSPESLLQLKALKPGVIQILGVKTSRFLCQRPDGALYGSLHFDPEACS | |
| FRELLLEDGYNVYQSEAHGLPLHLPGQKSPHRDPAPRGPARFLPLPG | |
| LPPALPEPPGILAPQPPDVGSSDPLSLVGGSQGRSPSYAS | |
| HGEGTFTSDVSSYLEEQAAKCFIAWLVKGGGGAQPGAQPGAQPGAQ | SEQ ID NO: 22 |
| PAHPIPDSSPLLQFGGQVRQRYLYTDDAQQTEAHLEIREDGTVGGAA | |
| DQSPESLLQLKALKPGVIQILGVKTSRFLCQRPDGALYGSLHFDPEACS | |
| FRELLLEDGYNVYQSEAHGLPLHLPGQKSPHRDPAPRGPARFLPLPG | |
| LPPALPEPPGILAPQPPDVGSSDPLSLVGGSQGRSPSYES | |
| HGEGTFTSDVSSYLEEQAARCFIAWLVKGGGGAQPGAQPGAQPGAQ | SEQ ID NO: 23 |
| PAHPIPDSSPLLQFGGQVRQRYLYTDDAQQTEAHLEIREDGTVGGAA | |
| DQSPESLLQLKALKPGVIQILGVKTSRFLCQRPDGALYGSLHFDPEACS | |
| FRELLLEDGYNVYQSEAHGLPLHLPGQKSPHRDPAPRGPARFLPLPG | |
| LPPALPEPPGILAPQPPDVGSSDPLSLVGGSQGRSPSYES | |
| HGEGTFTSDVSSYLEEQAAKCFIAWLVKGGGGAQPGAQPGAQPGAQ | SEQ ID NO: 24 |
| PGAQPGAQPGAQPGAQPAHPIPDSSPLLQFGGQVRQRYLYTDDAQQ | |
| TEAHLEIREDGTVGGAADQSPESLLQLKALKPGVIQILGVKTSRFLCQR | |
| PDGALYGSLHFDPEACSFRELLLEDGYNVYQSEAHGLPLHLPGQKSP | |
| HRDPAPRGPARFLPLPGLPPALPEPPGILAPQPPDVGSSDPLSLVGGS | |
| QGRSPSYAS | |
| HGEGTFTSDVSSYLEEQAAKCFIAWLVKGGGGAQPGAQPGAQPGAQ | SEQ ID NO: 25 |
| PGAQPGAQPGAQPGAQPAHPIPDSSPLLQFGGQVRQRYLYTDDAQQ | |
| TEAHLEIREDGTVGGAADQSPESLLQLKALKPGVIQILGVKTSRFLCQR | |
| PDGALYGSLHFDPEACSFRELLLEDGYNVYQSEAHGLPLHLPGQKSP | |
| HRDPAPRGPARFLPLPGLPPALPEPPGILAPQPPDVGSSDPLSLVGGS | |
| QGRSPSYES | |
| HGEGTFTSDVSSYLEEQAAKCFIAWLVKGGGGAQPGAQPGAQPGAQ | SEQ ID NO: 26 |
| PGAQPGAQPGAQPGAQPGAQPGAQPGAQPGAQPGAQPGAQPGAQ | |
| PGAQPAHPIPDSSPLLQFGGQVRQRYLYTDDAQQTEAHLEIREDGTV | |
| GGAADQSPESLLQLKALKPGVIQILGVKTSRFLCQRPDGALYGSLHFD | |
| PEACSFRELLLEDGYNVYQSEAHGLPLHLPGQKSPHRDPAPRGPARF | |
| LPLPGLPPALPEPPGILAPQPPDVGSSDPLSLVGGSQGRSPSYES | |
| HGEGTFTSDVSSYLEEQAAKCFIAWLVKGGGGAQPGAQPGAQPGAQ | SEQ ID NO: 27 |
| PGAQPGAQPGAQPGAQPGAQPGAQPGAQPGAQPGAQPGAQPGAQ | |
| PGAQPGAQPGAQPGAQPGAQPGAQPGAQPGAQPGAQPGAQPGAQ | |
| PGAQPGAQPGAQPGAQPGAQPGAQPAHPIPDSSPLLQFGGQVRQRY | |
| LYTDDAQQTEAHLEIREDGTVGGAADQSPESLLQLKALKPGVIQILGVK | |
| TSRFLCQRPDGALYGSLHFDPEACSFRELLLEDGYNVYQSEAHGLPL | |
| HLPGQKSPHRDPAPRGPARFLPLPGLPPALPEPPGILAPQPPDVGSSD | |
| PLSLVGGSQGRSPSYES | |
| HGEGTFTSDVSSYLEEQAAREFIAWLVKGCGGAQPGAQPGAQPGAQ | SEQ ID NO: 28 |
| PAHPIPDSSPLLQFGGQVRQRYLYTDDAQQTEAHLEIREDGTVGGAA | |
| DQSPESLLQLKALKPGVIQILGVKTSRFLCQRPDGALYGSLHFDPEACS | |
| FRELLLEDGYNVYQSEAHGLPLHLPGQKSPHRDPAPRGPARFLPLPG | |
| LPPALPEPPGILAPQPPDVGSSDPLSLVGGSQGRSPSYES | |
| HGEGTFTSDVSSYLEEQAAKEFIAWLVKGGGGAQPGAQPCGAQPGA | SEQ ID NO: 29 |
| QPAHPIPDSSPLLQFGGQVRQRYLYTDDAQQTEAHLEIREDGTVGGA | |
| ADQSPESLLQLKALKPGVIQILGVKTSRFLCQRPDGALYGSLHFDPEAC | |
| SFRELLLEDGYNVYQSEAHGLPLHLPGQKSPHRDPAPRGPARFLPLP | |
| GLPPALPEPPGILAPQPPDVGSSDPLSLVGGSQGRSPSYAS | |
| HGEGTFTSDVSSYLEEQAAREFIAWLVKGGGGAQPGAQPCGAQPAH | SEQ ID NO: 30 |
| PIPDSSPLLQFGGQVRQRYLYTDDAQQTEAHLEIREDGTVGGAADQS | |
| PESLLQLKALKPGVIQILGVKTSRFLCQRPDGALYGSLHFDPEACSFRE | |
| LLLEDGYNVYQSEAHGLPLHLPGQKSPHRDPAPRGPARFLPLPGLPP | |
| ALPEPPGILAPQPPDVGSSDPLSLVGGSQGRSPSYAS | |
| HGEGTFTSDVSSYLEEQAACEFIAWLVKGGGGAQPGAQPGAQPGAQ | SEQ ID NO: 31 |
| PAHPIPDSSPLLQFGGQVRQRYLYTDDAQQTEAHLEIREDGTVGGAA | |
| DQSPESLLQLKALKPGVIQILGVKTSRFLCQRPDGALYGSLHFDPEACS | |
| FRELLLEDGYNVYQSEAHGLPLHLPGQKSPHRDPAPRGPARFLPLPG | |
| LPPALPEPPGILAPQPPDVGSSDPLSLVGPSQGRSPSYCS | |
| HGEGTFTSDVSSYLEEQAAKCFIAWLVKGGGGAQPGAQPGAQPGAQ | SEQ ID NO: 32 |
| PAHPIPDSSPLLQFGGQVRQRYLYTDDAQQTEAHLEIREDGTVGGAA | |
| DQSPESLLQLKALKPGVIQILGVKTSRFLCQRPDGALYGSLHFDPEACS | |
| FRELLLEDGYNVYQSEAHGLPLHLPGQKSPHRDPAPRGPARFLPLPG | |
| LPPALPEPPGILAPQPPDVGSSDPLSLVGPSQGRSPSYCS | |
| HGEGTFTSDVSSYLEEQAARCFIAWLVKGGGGAQPGAQPGAQPGAQ | SEQ ID NO: 33 |
| PAHPIPDSSPLLQFGGQVRQRYLYTDDAQQTEAHLEIREDGTVGGAA | |
| DQSPESLLQLKALKPGVIQILGVKTSRFLCQRPDGALYGSLHFDPEACS | |
| FRELLLEDGYNVYQSEAHGLPLHLPGQKSPHRDPAPRGPARFLPLPG | |
| LPPALPEPPGILAPQPPDVGSSDPLSLVGPSQGRSPSYCS | |
| HGEGTFTSDVSSYLEEQAAREFIAWLVKGGGGAQPGAQPCGAQPGA | SEQ ID NO: 34 |
| QPAHPIPDSSPLLQFGGQVRQRYLYTDDAQQTEAHLEIREDGTVGGA | |
| ADQSPESLLQLKALKPGVIQILGVKTSRFLCQRPDGALYGSLHFDPEAC | |
| SFRELLLEDGYNVYQSEAHGLPLHLPGQKSPHRDPAPRGPARFLPLP | |
| GLPPALPEPPGILAPQPPDVGSSDPLSLVGPSQGRSPSYCS | |

### Fusion Compounds

The term “fusion compound” refers to a derivative of a fusion protein. In some embodiments, a fusion compound comprises a fusion protein and a substituent. A “GLP-1/FGF21 fusion compound” is a derivative of a GLP-1/FGF21 fusion protein. In some embodiments, the compounds of the invention are GLP-1/FGF21 fusion compounds. The fusion compound may e.g. be referred to with reference to the GLP-1 polypeptides, the spacer, the FGF21 polypeptide, and the substituent. An example of the fusion compound nomenclature used herein is: S{Beta-27}-[2-[2-[[2-[2-[2-[[2-[2-[2-[[(4S)-4-carboxy-4-(17-carboxyheptadecanoyl amino)butanoyl]amino]ethoxy]ethoxy]acetyl]amino]ethoxy]ethoxy]acetyl]amino]-ethylamino]-2-oxoethyl]-[8G, 22E, 270, 36G]GLP-1(7-37)-(GAQP)x32, A-[121Q, 168L, 171G, 180E]FGF21 (1-181). In this example the fusion compound consists of a GLP-1 polypeptide of the formula [8G, 22E, 270, 36G]GLP-1 (7-37), and a FGF21 polypeptide of the formula [121Q, 168L, 171G, 180E]FGF21 (1-181), wherein the GLP-1 polypeptide and FGF21 polypeptide are separated by a spacer of formula (GAQP)x32, A, and wherein a substituent of formula S{Beta-27}-[2-[2-[[2-[2-[2-[[2-[2-[2-[[(4S)-4-carboxy-4-(17-carboxyheptadecanoyl amino)butanoyl]amino]ethoxy]ethoxy]acetyl]amino]ethoxy]ethoxy]acetyl]amino]-ethylamino]-2-oxoethyl] is attached to the Cys residue in position 27 of the GLP-1 polypeptide.

In some embodiments, the fusion compound comprises a fusion protein according to A-B-C(Chem. 1), wherein A is a GLP-1 polypeptide, which is an analogue of GLP-1 (7-37) (SEQ ID NO: 1); B is a peptide spacer comprising or consisting of 1-257 amino acids; and C is a FGF21 polypeptide analogue, which is an analogue of FGF21 (1-180) (SEQ ID NO: 2); and at least of substituent.

In some embodiments, the fusion compound has an improved half-life. In some embodiments, the fusion compound is a bifunctional fusion compound. In some embodiments, the fusion compound comprises a substituent. In some embodiments, the fusion compound comprises one or more substituents, such as two substituents or four substituents. In some embodiments, the fusion compound comprises a substituent attached to the Cys residue at a position corresponding to position 27 of GLP-1(7-37) (SEQ ID NO:1).

In some embodiments, the fusion compound comprises a substituent attached to the Cys residue at a position corresponding to position 26 of GLP-1(7-37) (SEQ ID NO:1). In some embodiments, the fusion compound comprises a substituent attached to the Cys residue at a position corresponding to position 36 of GLP-1(7-37) (SEQ ID NO:1). In some embodiments, the fusion compound comprises a substituent attached to the Cys residue of the spacer. In some embodiments, the fusion compound comprises a substituent attached to the Cys residue at a position corresponding to position 180 of FGF21(1-180) (SEQ ID NO:2). In some embodiment, the fusion compound comprises a first substituent attached to a Cys residue of the GLP-1 polypeptide moiety of the fusion compound, such as to the Cys residue at a position corresponding to position 26 of GLP-1(7-37) (SEQ ID NO:1) or such as to the Cys residue at a position corresponding to position 27 of GLP-1(7-37) (SEQ ID NO:1), and a second substituent attached to the Cys residue at a position corresponding to position 180 of FGF21(1-180) (SEQ ID NO:2). In some embodiment, the fusion compound comprises a first substituent attached to a Cys residue of the spacer moiety of the fusion compound and a second substituent attached to a Cys residue of the FGF21 polypeptide moiety of the fusion compound, where said Cys is at a position corresponding to position 180 of FGF21(1-180) (SEQ ID NO:2)

In some embodiments, the fusion compound is selected from a list consisting of Chem. 8, Chem. 9, Chem. 10, Chem. 11, Chem. 12, Chem. 13, Chem. 14, Chem. 15, Chem. 16, Chem. 17, Chem. 18, Chem. 19, Chem. 20, Chem. 21, Chem. 22, and Chem. 23.

In some embodiments, the fusion compound is selected form the list consisting of

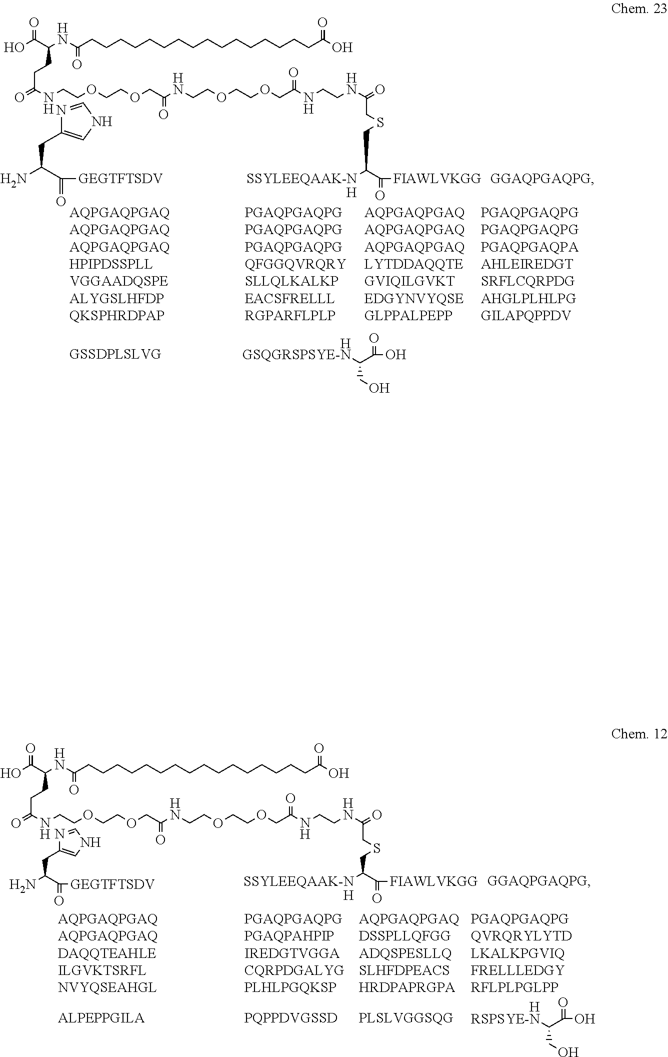

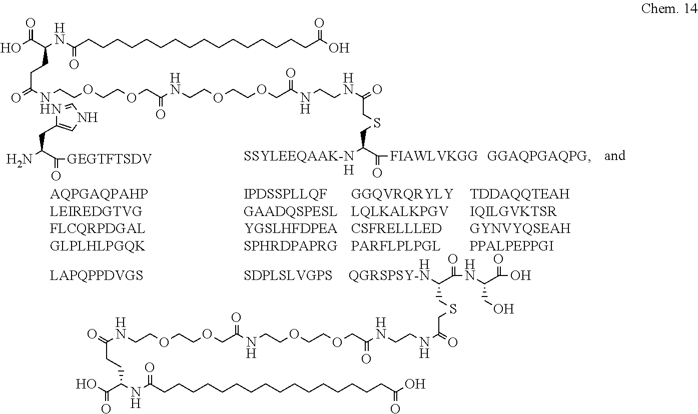

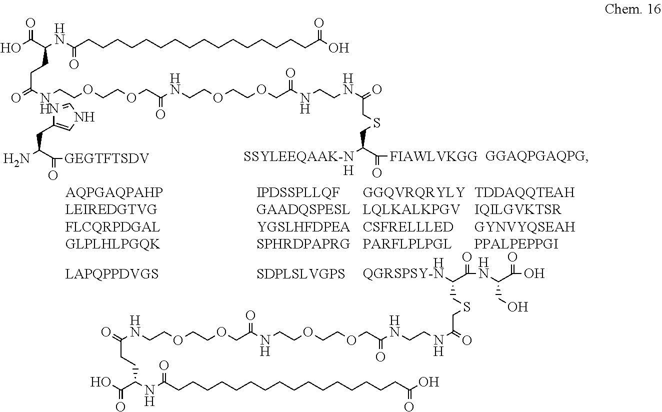

### Pharmaceutically Acceptable Salt, Amide, or Ester

The fusion compound may be in the form of a pharmaceutically acceptable salt, amide, or ester.

Salts are e.g. formed by a chemical reaction between a base and an acid, e.g.: 2NH₃+H₂SO₄→(NH₄)₂SO₄.

The salt may be a basic salt, an acid salt, or it may be neither nor (i.e. a neutral salt). Basic salts produce hydroxide ions and acid salts hydronium ions in water.

The salts of the fusion compound may be formed with added cations or anions between anionic or cationic groups, respectively. These groups may be situated in the polypeptide backbone and/or in the substituent of the fusion compound.

Non-limiting examples of anionic groups of the fusion compounds of the invention include free carboxylic groups in the substituent, if any, as well as in the GLP-1 polypeptide and/or the FGF21 polypeptide. The amino acid sequence often includes a free carboxylic acid group at the C-terminus, and it may also include free carboxylic groups at internal acid amino acid residues such as Asp and Glu.

Non-limiting examples of cationic groups in the fusion compounds include the free amino group at the N-terminus, if present, as well as any free amino group of internal basic amino acid residues such as His, Arg, and Lys. The amino group at the N-terminus of the fusion compounds of the invention may be free or acetylated.

The ester of the derivatives of the invention may, e.g., be formed by the reaction of a free carboxylic acid group with an alcohol or a phenol, which leads to replacement of at least one hydroxyl group by an alkoxy or aryloxy group The ester formation may involve the free carboxylic group at the C-terminus of the polypeptide backbone, and/or any free carboxylic group in the substituent.

The amide of the derivatives of the invention may, e.g., be formed by the reaction of a free carboxylic acid group with an amine or a substituted amine, or by reaction of a free or substituted amino group with a carboxylic acid.

The amide formation may involve the free carboxylic group at the C-terminus of the polypeptide backbone, any free carboxylic group in the substituent, the amino group at the N-terminus of the polypeptide backbone, and/or any amino group in the substituent.

In a one embodiment, the fusion compound is in the form of a pharmaceutically acceptable salt. In one embodiment, the fusion compound is in the form of a pharmaceutically acceptable amide. In one embodiment, the fusion compound is in the form a pharmaceutically acceptable ester.

### GLP-1 Activity

In a functional aspect of the invention, the fusion compound has GLP-1 activity. The term “GLP-1 activity” as used herein, refers to the capability to activate of the GLP-1 receptor, and this activation may also be referred to as “potency”.

In some embodiments, the term refers to the agonistic activity/potency in vivo. In some embodiments, activation of the GLP-1 receptor is determined by measuring the cAMP response of cells stably expressing GLP-1 receptor upon contact with the agonist in vitro. In some embodiments, the cells according to Example 2. In some embodiments, the GLP-1 receptor is human GLP-1 receptor. The GLP-1 activity may be expressed as an EC₅₀ value or an EC₅₀ value relative to that of a reference compound with GLP-1 activity, e.g. Chem. 24. The GLP-1 activity may be measured in the presence of HSA. The GLP-1 activity is preferably determined as described in Example 2.

In some embodiments, the GLP-1 activity is measured in Baby Hamster Kidney (BHK) cells stably expressing the human GLP-1 receptor together with a CRE-luciferase reporter gene enabling indirect measure of cAMP formation by adenylate cyclase. In some embodiments, the GLP-1 activity is measured in the presence of 1% HSA. In some embodiments the GLP-1 activity is measured as the activation of GLP-1R without the presence of HSA as described in “general method for measuring GLP-1 activity”/example 2 and expressed as the EC₅₀ value, wherein the EC₅₀ is below 100 pM, preferably below 70 pM, preferably below 50 pM, preferably below 20 pM.

### FGF21 Activity

In a functional aspect of the invention, the fusion compound has FGF21 activity. The term FGF21 activity, as used herein, refers to the capability to activate a FGFR complex, and this activation may also be referred to as “potency”. The activity may, e.g. be determined in vitro using in an assay HEK293 cells endogenously express several FGF receptors, including FGFR1c, FGFR3c, and BKL. For example, the response of the human FGFR may be measured using HEK (Human Embryonic Kidney cells) overexpressing human beta-klotho (BKL). The FGF21 activity may be expressed as an EC₅₀ value. The FGF21 activity may be measured in the presence of HSA. The FGF21 activity is preferably determined as described in Example 3.

In some embodiment, the FGF21 activity is measured in a HEK293 cell line overexpressing FGFR1c and human beta-klotho receptor (BKL). In some embodiments, the FGF21 activity is measured in the presence of 0.1% HSA. In some embodiments the FGF21 activity is measured without the presence of HSA as the activation of FGFR complex as described in Example 2 and expressed as the EC₅₀ value, wherein the EC₅₀ is below 50 nM, preferably below 10 nM, preferably below 5 nM, preferably 2 nM.

### Pharmacokinetics

In some embodiments, a fusion compound according to the invention may have an increased mean residence time “MRT” compared to native FGF21 and native GLP-1, respectively.

In some embodiments, the MRT may be measured in vivo using mice. The MRT measured in vivo using mice is preferably determined as described in Example 4.1. In some embodiments, the MRT may be measured in vivo using minipigs. The MRT measured in vivo using minipigs is preferably determined as described in Example 4.2. In some embodiments, the MRT may be measured in vivo using cynomolgus monkey. The MRT measured in cynomolgus monkeys is preferably determined as described in Example 4.3.

In some embodiments, the half-life of the fusion compound is calculated using “individual best fit” of the log-linear regression of concentrations versus time. In some embodiments, the MRT is calculated as MRT=AUMC/AUC, where AUC is AUMC is calculated by the trapeziodal methods. In some embodiments, the MRT of the fusion compound is calculated based on a non-compartmental analysis. In some embodiments, the MRT of the fusion compound is at least 2.5 hours, preferably, preferably at least 3 hours, preferably at least 4 hours, preferably at least 5 hours, preferably at least 6 hours, preferably at least 7 hours, more preferably at least 8 hours, preferably at least 9 hours, preferably at least 9.4 hours when measured in mice according to Example 4.1. In some embodiments, the MRT of the fusion compound is at least 50 hours, preferably at least 60 hours, preferably at least 70 hours, preferably at least 80 hours, preferably at least 90 hours when measured in mini pigs according to Example 4.2 In some embodiments, the MRT of the fusion compound is at least 30 hours, preferably at least 40 hours, preferably at least 50 hours, preferably at least 52 hours when measured in cynomolgus monkeys according to Example 4.3.

### Pharmacodynamics

FGF21 is a human hormone synthesised in the liver and involved in glucose, lipid and energy homeostasis. Treatment with FGF21 efficiently lowers triglycerides, LDL-C and VLDL-C, while it increases HDL-C. Thus, a skilled person would expect that a compound with FGF21 activity is capable of lowering plasma levels of triglycerides, LDL-C and VLDL-C. Treatment of obese rodents with FGF21 efficiently lowers body weight, which correlates with the IGF-1, liver triglycerides and liver enzymes as ALT and AST. Thus, the PD effect o FGF21 can be studied in obese animal models

GLP-1 is an incretin secreted in the intestine involved in glucose metabolism and satiety. Treatment with GLP-1 acutely and efficiently reduces food intake and reduces body weight.

In one embodiment the fusion compounds are capable of lowering acute food intake as measured in mice. In one embodiment the fusion compounds are capable of efficiently lowering body weight as measured in mice. In one embodiment fusion compounds are capable of lowering liver triglycerides as measured in DIO mice or DIO-NASH-model (GAN diet). In one embodiment fusion compounds are capable of lowering liver enzymes like ALT as measured in DIO mice or DIO-NASH-model (GAN diet).

In a functional aspect of the invention, the fusion compound may be capable of lowering plasma concentrations of low-density lipoprotein cholesterol (LDL-C). Also or alternatively, the fusions compounds of the invention may be capable of lowering plasma levels of triglycerides. Also or alternatively, the fusions compounds of the invention are may be capable of lowering plasma levels of total cholesterol.

### Pharmaceutical Indication/Medical Uses

The present invention also relates to the fusion compound for use as a medicament. The term “treatment”, as used herein, refers to the medical treatment of any human subject in need thereof. The treatment may be preventive, prophylactic, palliative, symptomatic and/or curative. The timing and purpose of said treatment may vary from one individual to another, according to the status of the subject's health.

According to a second aspect of the invention there is provided a fusion compound, as hereinbefore defined (i.e. a compound as defined in the first aspect of the invention, including all embodiments and particular features thereof), for use as a pharmaceutical (or for use in medicine).

For the avoidance of doubt, references to compounds as defined in the first aspect of the invention will include references to fusion proteins of Chem. 1 (including all embodiments thereof) and pharmaceutically acceptable salts, esters, and amides thereof.

In some embodiments, the fusion compound may be particularly useful in treating and/or preventing eating disorders, cardiovascular diseases, diabetic complications; and/or for improving lipid parameters such as prevention and/or treatment of dyslipidemia, lowering total serum lipids; increasing HDL; lowering small, dense LDL; lowering VLDL; lowering triglycerides; lowering cholesterol; lowering plasma levels of lipoprotein a (Lp(a)) in a human; increasing plasma adiponectin in a human; inhibiting generation of apolipoprotein A (apo(A)), improving β-cell function; and/or for delaying or preventing diabetic disease progression; and/or for of treatment and/or prevention of hepatic steatosis, non-alcoholic fatty liver disease (NAFLD), metabolic dysfunction-associated steatotic liver disease (MAFLD), alcohol related liver disease (ALD), MetALD, and non-alcoholic steatohepatitis (NASH), metabolic dysfunction-associated steatohepatitis (MASH).

MASH is proposed as the replacement term for NASH describing the same diagnostic entity (M. E. Rinella, et al, j.aohep.2023.101133). As used herein the terms “NASH” and MASH mean the same thing and are used interchangeably.

MAFLD is proposed as the replacement term for NAFLD describing the same diagnostic entity (M. E. Rinella, et al, j.aohep.2023.101133). As used herein the terms “NAFDL” and “MAFLD” means the same thing and are used interchangeably.

In a third aspect of the invention, there is provided a compound of the invention, as hereinbefore defined, for use in the treatment in the treatment and/or prevention of a disease. The disease may be selected from the group consisting of diabetes and related diseases, such as eating disorders, cardiovascular diseases, diabetic complications; and/or for improving lipid parameters, improving β-cell function such as prevention and/or treatment of dyslipidemia, lowering total serum lipids; increasing HDL; lowering small, dense LDL; lowering VLDL; lowering triglycerides; lowering cholesterol; lowering plasma levels of lipoprotein a (Lp(a)) in a human; inhibiting generation of apolipoprotein A (apo(A)); and/or for delaying or preventing diabetic disease progression; and/or for of treatment and/or prevention of hepatic steatosis, NAFDL, and NASH.

In an alternative third aspect of the invention, there is provided a method of treating and/or preventing a disease, such as diabetes and related diseases, such as eating disorders, cardiovascular diseases, diabetic complications; and/or for improving lipid parameters such as prevention and/or treatment of dyslipidemia, lowering total serum lipids; increasing HDL; lowering small, dense LDL; lowering VLDL; lowering triglycerides; lowering cholesterol; lowering plasma levels of lipoprotein a (Lp(a)) in a human; inhibiting generation of apolipoprotein A (apo(A)), improving β-cell function; and/or for delaying or preventing diabetic disease progression; and/or for of treatment and/or prevention of hepatic steatosis, NAFDL, and NASH, comprising administering to a patient in need thereof a therapeutically effective amount of a compound of the invention, as hereinbefore defined.

In a further alternative third aspect of the invention, there is provided the use of a compound of the invention, as hereinbefore defined, for the manufacture of a medicament for the treatment or prevention of a disease such as eating disorders, cardiovascular diseases, diabetic complications; and/or for improving lipid parameters such as prevention and/or treatment of dyslipidemia, lowering total serum lipids; increasing HDL; lowering small, dense LDL; lowering VLDL; lowering triglycerides; lowering cholesterol; lowering plasma levels of lipoprotein a (Lp(a)) in a human; inhibiting generation of apolipoprotein A (apo(A)), improving β-cell function; and/or for delaying or preventing diabetic disease progression; and/or for of treatment and/or prevention of hepatic steatosis, NAFDL, and NASH.

In some embodiments, compounds of the invention may be used for the following medical treatments:
(i) prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1C;
(ii) delaying or preventing diabetic disease progression, such as progression in type 2 diabetes, delaying the progression of impaired glucose tolerance (IGT) to insulin requiring type 2 diabetes, delaying or preventing insulin resistance, and/or delaying the progression of non-insulin requiring type 2 diabetes to insulin requiring type 2 diabetes;
(iii) prevention and/or treatment of eating disorders, such as obesity, e.g. by decreasing food intake, reducing body weight, suppressing appetite, inducing satiety; treating or preventing binge eating disorder, bulimia nervosa, and/or obesity induced by administration of an antipsychotic or a steroid; reduction of gastric motility; delaying gastric emptying; increasing physical mobility; and/or prevention and/or treatment of comorbidities to obesity, such as osteoarthritis and/or urine incontinence;
(iv) weight maintenance after successful weight loss (either drug induced or by diet and exercise)—i.e. prevention of weight gain after successful weight loss.
(v) prevention and/or treatment of liver disorders, such as hepatic steatosis, non-alcoholic fatty liver disease (NAFLD), non-alcoholic steatohepatitis (NASH), liver inflammation or fatty liver.

In some embodiments the invention relates to compounds of the invention for use in the prevention and/or treatment acute and/or chronic pancreatitis.

In some embodiment, the invention relates to compounds of the invention for use in the prevention and/or treatment of a disease selected from the group consisting of type 2 diabetes, metabolic syndrome, obesity, insulin resistance, as well as pre diabetes, diabetic retinopathy, diabetic neuropathy, diabetic nephropathy, chronic kidney disease, diabetic kidney disease, diabetic dyslipidemia, fatty liver disease including non-alcoholic fatty liver disease (NASH), and atherosclerosis.

In some embodiments, the invention relates to compounds of the invention for use in the prevention and/or treatment of heptocellular carcinoma (HCC).

In some embodiments, the invention relates to compounds of the invention for use in the prevention and/or treatment of alcohol steathepatitis (ASH) or alcoholic fatty liver disease (AFDL).

In some embodiments, the invention relates to compounds of the invention for use in the prevention and/or treatment of NASH, wherein said use prevents and/or delays increase in the relative liver weight, plasma alanine aminotransferase levels, liver triglyceride content and/or liver cholesterol. The relative liver weight is defined as liver weight as percentage of total body weight. In some embodiments said use reduces the relative liver weight, plasma alanine aminotransferase levels, liver triglyceride content and/or liver cholesterol.

In some embodiments the invention relates to compounds of the invention for use in the prevention and/or treatment of NASH, wherein said use prevents, delays and/or reduces any histopathological signs of steatosis.

In some embodiments the invention relates to compounds of the invention for use in the prevention and/or treatment of NASH, wherein said use prevents, delays and/or reduces inflammation in the liver.

NASH is the most extreme form of NAFLD. NAFLD is one of the types of fatty liver which occurs when fat is deposited in the liver due to causes other than consumption of alcohol. The deposition of fat is also referred to as steatosis. Patients suffering from NASH frequently have obesity, type 2 diabetes mellitus, dyslipidemia, and/or the metabolic syndrome. Symptoms of NASH are fatigue, malaise, or right upper quadrant abdominal discomfort.

In some embodiments the invention relates to compounds of the invention for use in the prevention and/or treatment of NASH, wherein said use prevents, delays and/or reduces fibrogenesis in the liver. In some embodiments the present invention relates to a compound for use in the prevention and/or treatment of NASH, wherein said compound is administered in the form of a pharmaceutical composition comprising 1-50 mg/ml compounds, such as 5-40 mg/ml, such as 10-30 mg/ml compound.

In some embodiments the invention relates to compounds of the invention for use in the prevention and/or treatment of NASH, wherein said compound is subcutaneously administered once weekly.

In some embodiments the invention relates to compounds of the invention for use in the prevention and/or treatment of NASH, wherein said compound is subcutaneously administered every day or every second day or every third day or every fourth day or every fifth day or every sixth day. In some embodiments said compound is administered for at least 12 months.

In some embodiments the invention relates to compounds of the invention for use in the prevention and/or treatment of NASH, wherein said compound is administered in a therapeutically effective amount to a subject in need thereof.

In some embodiments said subject is obese and/or has diabetes. In some embodiments said subject suffers from overweight, obesity, hyperglycemia, type 2 diabetes, impaired glucose tolerance and/or type 1 diabetes.

In some embodiment, the indication may be Type 2 diabetes and/or dyslipidemia and/or obesity. In some embodiments the invention relates to a method for weight management. In some embodiments the invention relates to a method for reduction of appetite. In some embodiments the invention relates to a method for reduction of food intake.

Generally, all subjects suffering from obesity are also considered to be suffering from overweight. In some embodiments the invention relates to a method for treatment or prevention of obesity. In some embodiments the invention relates to use of the derivative of the present invention for treatment or prevention of obesity. In some embodiments the subject suffering from obesity is human, such as an adult human or a paediatric human (including infants, children, and adolescents). Body mass index (BMI) is a measure of body fat based on height and weight. The formula for calculation is BMI=weight in kilograms/height in meters. A human subject suffering from obesity may have a BMI of >30; this subject may also be referred to as obese. In some embodiments the human subject suffering from obesity may have a BMI of >35 or a BMI in the range of >30 to <40. In some embodiments the obesity is severe obesity or morbid obesity, wherein the human subject may have a BMI of >40.

In some embodiments the invention relates to a method for treatment or prevention of overweight, optionally in the presence of at least one weight-related comorbidity. In some embodiments the invention relates to use of the compound of the invention for treatment or prevention of overweight, optionally in the presence of at least one weight-related comorbidity. In some embodiments the subject suffering from overweight is human, such as an adult human or a paediatric human (including infants, children, and adolescents). In some embodiments a human subject suffering from overweight may have a BMI of >25, such as a BMI of >27. In some embodiments a human subject suffering from overweight has a BMI in the range of 25 to <30 or in the range of 27 to <30. In some embodiments the weight-related comorbidity is selected from the group consisting of hypertension, diabetes (such as type 2 diabetes), dyslipidaemia, high cholesterol, and obstructive sleep apnoea.

In some embodiments the invention relates to a method for reduction of body weight. In some embodiments the invention relates to use of the compounds of the invention for reduction of body weight. A human to be subjected to reduction of body weight according to the present invention may have a BMI of >25, such as a BMI of >27 or a BMI of >30. In some embodiments the human to be subjected to reduction of body weight according to the present invention may have a BMI of >35 or a BMI of >40. The term “reduction of body weight” may include treatment or prevention of obesity and/or overweight.

The skilled person will understand that references to the treatment of a particular condition (or, similarly, to treating that condition) will take their normal meanings in the field of medicine. In particular, the terms may refer to achieving a reduction in the severity and/or frequency of occurrence of one or more clinical symptom associated with the condition, as adjudged by a physician attending a patient having or being susceptible to such symptoms. For example, in the case of NASH, the term may refer to increase in liver enzymes, increase in plasma lipids, increase in liver stiffness, increase in hepatic steatosis, loss of hepatic function, increase in liver toxicity.

As used herein, references to a patient (or to patients) will refer to a living subject being treated, including mammalian (e.g. human) patients. In particular, references to a patient will refer to human patients.

For the avoidance of doubt, the skilled person will understand that such treatment [or prevention] will be performed in a patient (or subject) in need thereof. The need of a patient (or subject) for such treatment [or prevention] may be assessed by those skilled the art using routine techniques. As used herein, the terms disease and disorder (and, similarly, the terms condition, illness, medical problem, and the like) may be used interchangeably. As used herein, the term effective amount will refer to an amount of a compound that confers a therapeutic effect on the treated patient. The effect may be observed in a manner that is objective (i.e. measurable by some test or marker) or subjective (i.e. the subject gives an indication of and/or feels an effect). In particular, the effect may be observed (e.g. measured) in a manner that is objective, using appropriate tests as known to those skilled in the art.

### Pharmaceutical Compositions

The present invention also relates to pharmaceutical compositions comprising the fusion compound. In one embodiment the pharmaceutical composition comprising the fusion compound comprises at least one pharmaceutically acceptable excipient. Pharmaceutical compositions/formulations as described herein may be prepared in accordance with standard and/or accepted pharmaceutical practice.

The term “excipient” broadly refers to any component other than the active therapeutic ingredient(s). The excipient may be an inert substance, an inactive substance, and/or a not medicinally active substance. The excipient may serve various purposes, e.g. as a carrier, vehicle, diluent, tablet aid, and/or to improve administration, and/or absorption of the active substance. The formulation of pharmaceutically active ingredients with various excipients is known in the art, see e.g. Remington: The Science and Practice of Pharmacy (e.g. 19th edition (1995), and any later editions). Additional, optional, ingredients of a pharmaceutical composition include, e.g., wetting agents, emulsifiers, antioxidants, bulking agents, metal ions, oily vehicles, proteins. Non-limiting examples of excipients are: Solvents, diluents, buffers, preservatives, tonicity regulating agents, chelating agents, surfactants, and stabilisers.

In a fourth aspect of the invention, there is provided a pharmaceutical composition comprising a fusion compound as hereinbefore defined and optionally one or more pharmaceutically acceptable excipients. Injectable compositions comprising can be prepared using the conventional techniques of the pharmaceutical industry which involve dissolving and mixing the ingredients as appropriate to give the desired end product. Thus, according to one procedure, the fusion compound is dissolved in a suitable buffer at a suitable pH so precipitation is minimised or avoided. In embodiments, the pharmaceutical compositions may include wetting agents, emulsifiers, antioxidants, bulking agents, tonicity modifiers, chelating agents, metal ions, oleaginous vehicles, proteins (e.g., human serum albumin, gelatine or proteins) or a zwitterion (e.g., an amino acid such as betaine, taurine, arginine, glycine, lysine and histidine). In some embodiments, the pharmaceutical composition comprises phosphate. In some embodiments, the pharmaceutical composition comprises propylene glycol and/or Tween 20. In some embodiments, the pharmaceutical composition comprises glycerol.

Parenteral administration may be performed by subcutaneous, intramuscular, intraperitoneal or intravenous injection by means of a syringe, optionally a pen-like syringe. Alternatively, parenteral administration can be performed by means of an infusion pump.

The pharmaceutical composition comprising the fusion compound may be of several dosage forms, e.g. a solution, a suspension, a tablet, and a capsule.

Thus, in a further aspect of the invention there is provided a process for the preparation of a pharmaceutical composition/formulation, as hereinbefore defined, which process comprises bringing into association the fusion compound, as hereinbefore defined, with one or more pharmaceutically-acceptable excipient.

Thus, in a fifth aspect of the invention, there is provided a pharmaceutical composition as defined in the fourth aspect of the invention for use in the treatment or prevention of a disease as defined herein, with reference to the third aspect of the invention and all embodiments thereof.

The pharmaceutical composition comprising the fusion compound may be administered to a patient in need thereof at several sites, e.g. at topical sites such as skin or mucosal sites; at sites which bypass absorption such as in an artery, in a vein, or in the heart; and at sites which involve absorption, such as in the skin, under the skin, in a muscle, orally, or in the abdomen.

The treatment with a fusion compound according to the present invention may also be combined with one or more additional pharmacologically active substances, such as amylin analogues or antifibrotics, such as SGLT2, such as FXR agonists, such as ACCi, such as THR beta agoists.

### Production Processes

The present invention also relates methods for producing the fusion compound. The preparation of polypeptides like GLP-1 analogues, FGF21 analogues, and spacers, is well known in the art. The polypeptides incorporated in the fusion compound (or fragments thereof), may for instance be prepared by classical recombinant methods, viz. by culturing a host cell containing a DNA sequence encoding the analogue and capable of expressing the polypeptide in a suitable nutrient medium under conditions permitting the expression of the polypeptide. Non-limiting examples of host cells suitable for expression of these polypeptides are: Escherichia coli, Saccharomyces cerevisiae, as well as mammalian BHK or CHO cell lines. Also, or alternatively, the polypeptides incorporated in the fusion compound (or fragments thereof), may be prepared by classical solid peptide synthesis, e.g. solid phase peptide synthesis using Boc or Fmoc chemistry or other well established techniques, see, e.g. Greene and Wuts, “Protective Groups in Organic Synthesis”, John Wiley & Sons, 1999, Florencio Zaragoza Dörwald, “Organic Synthesis on solid Phase”, Wiley-VCH Verlag GmbH, 2000, and “Fmoc Solid Phase Peptide Synthesis”, Edited by W. C. Chan and P. D. White, Oxford University Press, 2000. Fusion compound containing non-coded amino acids may be prepared as described in the art, e.g. Hodgson et al: “The synthesis of peptides and proteins containing non-natural amino acids”, Chemical Society Reviews, vol. 33, no. 7 (2004), p. 422-430.

In some embodiments, the fusion compounds of the invention are prepared in a stepwise manner: (i) recombinant preparation of the backbone polypeptide, and (ii) covalent attachment (e.g. by alkylation) of a substituent to the backbone polypeptide. Specific examples of methods of preparing the fusion compound are included in the experimental section.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### PARTICULAR EMBODIMENTS

The following are particular embodiments of the invention:
1. A fusion protein comprising a polypeptide of formula Chem. 1: A-B-C, wherein:
   A is a GLP-1 polypeptide, which is an analogue of SEQ ID NO:1;
   B is a spacer consisting of 1-257 amino acids;
   C is an FGF21 polypeptide, which is an analogue of SEQ ID NO: 2;
   or a pharmaceutically acceptable salt, amide or ester thereof.
2. The fusion protein according to embodiment 1, wherein the C-terminus of the GLP-1 polypeptide is fused to the N-terminus of the peptide spacer, and the C-terminus of the peptide spacer is fused to the N-terminus of the FGF21 analogue.
3. The fusion protein according to one of the preceding embodiments, wherein B consist of 5 to 257 amino acids.
4. The fusion protein according to one of the preceding embodiments, wherein B consist of 9 to 129 amino acids.
5. The fusion protein according to one of the preceding embodiments, wherein B comprises or consists of the formula Chem. 2 (GAQP)x-C_{y}-(GAQP)ₕ-Aⱼ, wherein x is an integer in the range of 0-64, y is an integer in the range of 0-1, h is an integer in the range of 1-64 and j is an integer in the range of 0-1.
6. The fusion protein according to one of the preceding embodiments, wherein B is selected from a list consisting of: SEQ ID NO: 13, SEQ ID NO: 14, SEQ ID NO: 15, SEQ ID NO: 16, SEQ ID NO: 17, and SEQ ID NO: 18.
7. The fusion protein according to one of the preceding embodiments, wherein B is SEQ ID NO: 13, SEQ ID NO: 16, or SEQ ID NO: 17.
8. The fusion protein according to one of the preceding embodiments, wherein A is at least 50%, preferably at least 55%, preferably at least 60%, preferably at least 65%, preferably at least 70%, preferably at least 75%, preferably at least 80%, preferably at least 85%, preferably at least 90%, or most preferably at least 95%, identical to SEQ ID NO: 1.
9. The fusion protein according to one of the preceding embodiments, wherein A comprises a maximum of 15 amino acid changes, preferably a maximum of 14 amino acid changes, preferably a maximum of 13 amino acid changes, preferably a maximum of 12 amino acid changes, preferably a maximum of 11 amino acid changes, preferably a maximum of 10 amino acid changes, preferably a maximum of 9 amino acid changes, preferably a maximum of 8 amino acid changes, preferably a maximum of 7 amino acid changes, preferably a maximum of 6 amino acid changes, preferably a maximum of 5 amino acid changes, and most preferably a maximum of 4 amino acid changes, as compared to SEQ ID NO: 1.
10. The fusion protein according to any preceding embodiment, wherein A comprises of consists of the amino acid sequence SEQ ID NO: 3.
11. The fusion protein according to any preceding embodiment, wherein A has 3-7 amino acid changes as compared to SEQ ID NO: 1.
12. The fusion protein according to any preceding embodiment, wherein A comprises a Cys residue at a position corresponding to position 26 or 27 or 36 of GLP-1(7-37) (SEQ ID NO:1), or optionally wherein A comprises an Arg residue at a position corresponding to position 26 of GLP-1(7-37) (SEQ ID NO:1)
13. The fusion protein according to any preceding embodiment, wherein A comprises the following amino acid changes as compared to GLP-1 (7-37) (SEQ ID NO: 1): [8G, 22E, 26R], [8G, 22E, 26C, 36G], [8G, 22E, 27C, 36G], [8G, 22E, 26R, 27C, 36G], [8G, 22E, 26R, 36C], [8G, 22E, 36G], or [8G, 22E, 26R, 36G].
14. The fusion protein according to any preceding embodiment, wherein A is selected from a list consisting of SEQ ID NO: 4, SEQ ID NO: 5, SEQ ID NO: 6, SEQ ID NO: 7, SEQ ID NO: 8, SEQ ID NO: 9, and SEQ ID NO: 35.
15. The fusion protein according to any preceding embodiment, wherein C is at least 50%, preferably at least 55%, preferably at least 60%, preferably at least 65%, preferably at least 70%, preferably at least 75%, preferably at least 80%, preferably at least 85%, preferably at least 90%, or most preferably at least 95%, identical to SEQ ID NO: 2.
16. The fusion protein according to any preceding embodiment, wherein C is comprises a maximum of 10 amino acid changes, preferably a maximum of 9 amino acid changes, preferably a maximum of 8 amino acid changes, preferably a maximum of 7 amino acid changes, preferably a maximum of 6 amino acid changes, preferably a maximum of 5 amino acid changes, preferably a maximum of 4 amino acid changes, most preferably a maximum of 3 amino acid changes, as compared to SEQ ID NO: 2.
17. The fusion protein according to any preceding embodiment, wherein C has 3 or 4 amino acid changes as compared to SEQ ID NO: 2.
18. The fusion protein according to any preceding embodiment, wherein C comprises 180C or 180E or 180A.
19. The fusion protein according to any preceding embodiment, wherein C comprises the following amino acid changes as compared to FGF21(1-181) (SEQ ID NO: 2): [121Q, 168L, 180C1 or [121Q, 168L, 171G] or [121Q, 168L, 171G, 180E].
20. The fusion protein according to any preceding embodiment, wherein C is selected from a list consisting of SEQ ID NO: 10, SEQ ID NO: 11, and SEQ ID NO: 12.
21. The fusion protein according to any preceding embodiment, wherein C has FGF21 activity.
22. The fusion protein according to any preceding embodiment, wherein C is capable of activating the FGFR1c.
23. The fusion protein according to any preceding embodiment, wherein the components of Chem. 1 are linked via amide bonds.
24. A fusion compound comprising a fusion protein according to any one of embodiments 1-23, wherein the fusion compound comprises one or more substituents.
25. The fusion compound according to embodiment 24, wherein the substituent comprises a protracting moiety.
26. The fusion compound according to embodiment 24 or embodiment 25, wherein the protracting moiety is a functional group (FG) with pKa<7.0.
27. The fusion compound according to any one of embodiments 24-26, wherein the protracting moiety is lipophilic, and/or negatively charged at physiological pH (7.4).
28. The fusion compound according to any one of embodiments 24-27, wherein the protracting moiety is a fatty acid.
29. The fusion compound according to any one of embodiments 24-28, wherein the protracting moiety wherein the protracting moiety is Chem. 3: HOOC—(CH₂)ₓ—CO—*, wherein x is an integer in the range of 10-20
30. The fusion compound according to embodiment 29, wherein the x of Chem. 3 is 12-20.
31. The fusion compound according to embodiment 29 or embodiment 30, wherein the x of Chem. 3 is 14-20
32. The fusion compound according to any one of embodiments 29-31, wherein the x of Chem. 3 is 14-18.
33. The fusion compound according to any one of embodiments 29-32, wherein the x of Chem. 3 is 16-20
34. The fusion compound according to any one of embodiments 29-32, wherein the x of Chem. 3 is 16-18.
35. The fusion compound according to any one of embodiments 29-34, wherein the x of Chem. 3 is 16.
36. The fusion compound according to any one of embodiments 24-35, wherein the substituent comprises a linker.
37. The fusion compound according to any one of embodiments 24-36, wherein the substituent a protracting moiety and a linker.
38. The fusion compound according to any one of embodiments 36-37, wherein the linker comprises at least one of each of Chem. 4, Chem. 5 and Chem. 6;
wherein Chem. 4 is *—NH—CH(COOH)—(CH₂)₂—CO—*,
wherein Chem. 5 is *—NH—(CH₂)₂—[O—(CH₂)₂]ₖ—O—[CH₂]ₘ—CO—*, wherein k is an integer in the range of 1 to 5, wherein m is an integer in the range of 1 to 5,
wherein Chem. 6 is *—NH—(CH₂)n-NH—CO—CH₂—*, wherein n is an integer in the range of 1 to 5,
wherein Chem. 4, Chem. 5, and Chem. 6 are interconnected via amide bonds and in the sequence indicated, connected at its *—NH end to the CO—* end of the protractor (Chem. 3), and at its CH₂—* end to an amino acid of A, B or C.
39. The fusion compound according to embodiment 38, wherein the k of Chem. 5 is 1.
40. The fusion compound according to embodiment 38 or embodiment 39, wherein the m of Chem. 5 is 1.
41. The fusion compound according to any one of embodiments 38-40, wherein wherein the n of Chem 6 is 2.
42. The fusion compound according to any one of embodiments 38-41, wherein the linker elements are linked via amide bonds.
43. The fusion compound according to any one of embodiments 38-42, wherein the linker comprises one element of Chem 4, two elements of Chem 5 and one element of Chem 6.
44. The fusion compound according to any one of embodiments 38-42, wherein the linker consists of one element of Chem 4, two elements of Chem 5 and one element of Chem 6.
45. The fusion compound according to any one of embodiments 38-43, wherein the protracting moiety is attached to Chem 4 of the linker, and wherein Chem 5 of the linker is attached to the polypeptide backbone.
46. The fusion compound according to any one of embodiments 38-44, wherein the substituent is Chem. 7:

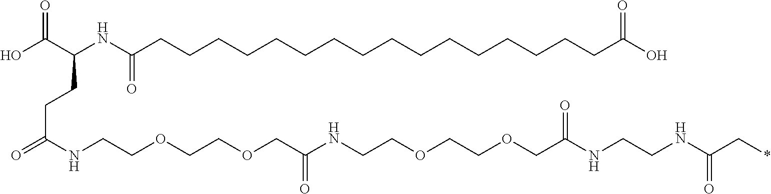 [SMILES: *CC(=O)NCCNC(=O)COCCOCCNC(=O)COCCOCCNC(=O)CC[C@H](NC(=O)CCCCCCCCCCCCCCCCC(=O)O)C(=O)O]

47. The fusion compound according to any one of embodiments 24-46, wherein the substituent is [2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl].
48. The fusion compound according to any one of embodiments 24 to 47, wherein the fusion compound comprises a first and a second substituent.
49. The fusion compound according to any one of embodiments 24 to 48, wherein at least one substituent is attached to any one of the amino acid residues in A, B or C.
50. The fusion compound according to any one of embodiments 24 to 49, wherein at least one substituent is attached to an amino acid residue of A.
51. The fusion compound according to any one of embodiments 24 to 50, wherein at least one substituent is attached to an amino acid residue of B.
52. The fusion compound according to any one of embodiments 24 to 51, wherein at least one substituent is attached to an amino acid residue of C.
53. The fusion compound according to any one of embodiment 48 or embodiment 49, wherein the first substituent is attached to A or B and the second substituent is attached to C.
54. The fusion compound according to any one of embodiments 24-53, wherein the substituent is attached to a Cys residue.
55. The fusion compound according to any one of embodiments 24-54, wherein the substituent is attached to a Cys residue in A or B or C.
56. The fusion compound according to any one of embodiments 24-55, wherein A comprises 26C or 27C or 36C.
57. The fusion compound according to any one of embodiments 24-55, wherein C comprises 180C.
58. The fusion compound according to any one of embodiments 24-56, wherein the substituent is attached to 26C or 27C or 36C in A.
59. The fusion compound according to any one of embodiments 24-55 or 57, wherein the substituent is attached to 180C in C.
60. The fusion compound according to any one of embodiments 24-59, wherein the fusion compound is selected from a list consisting of

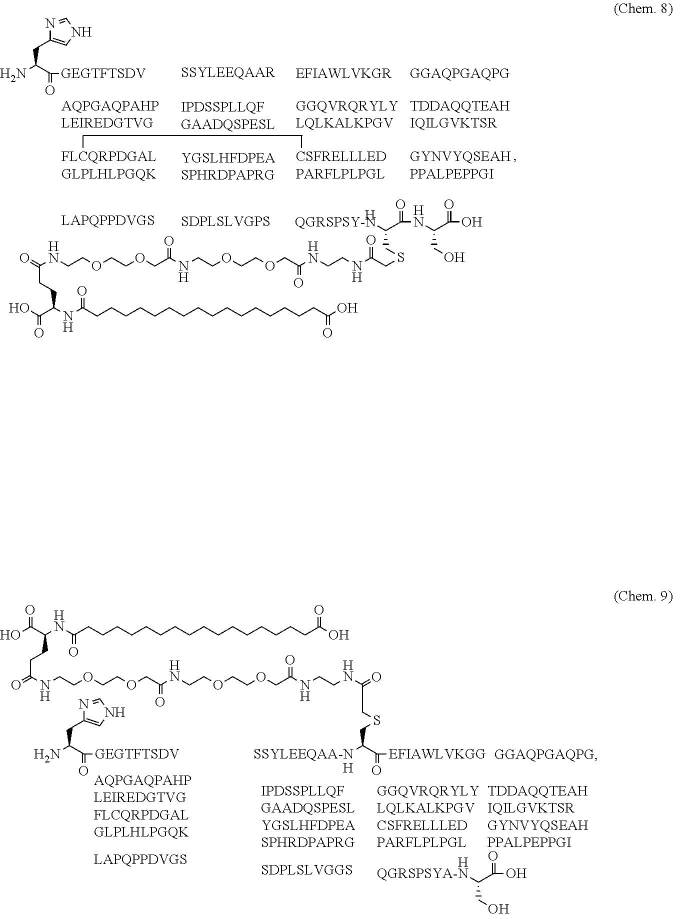

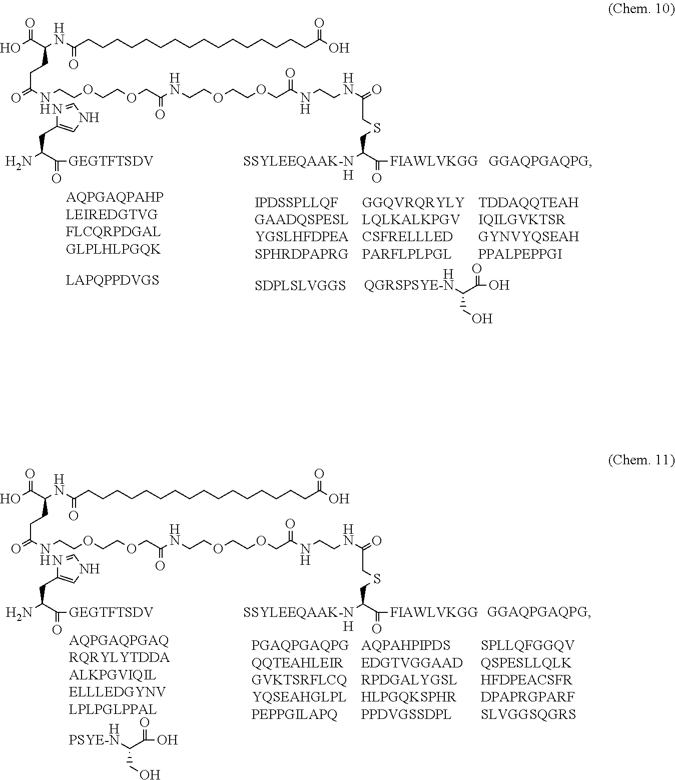

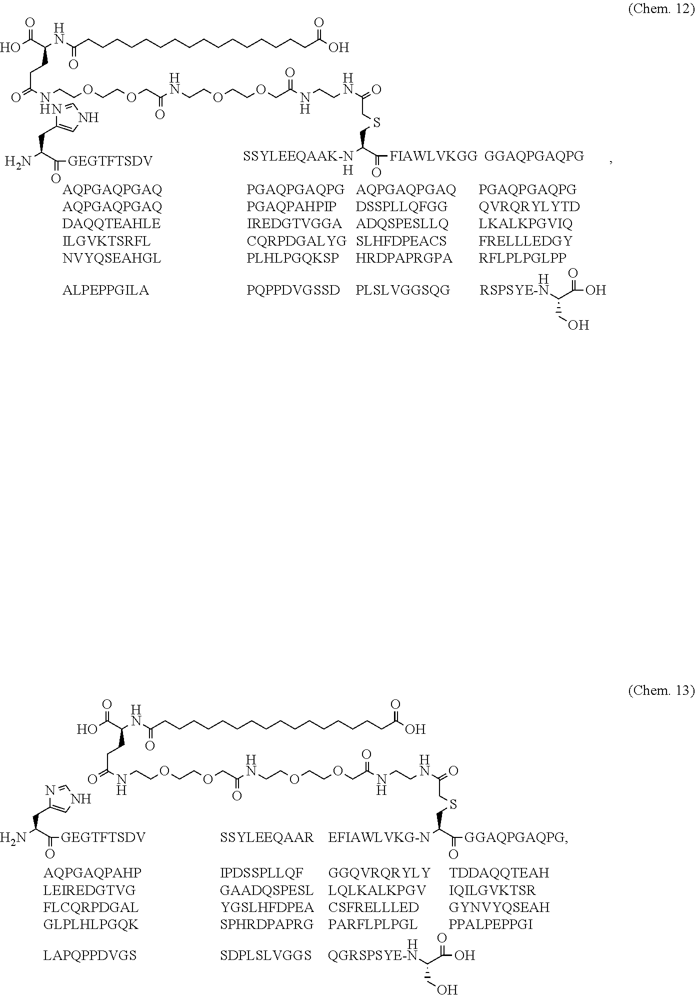

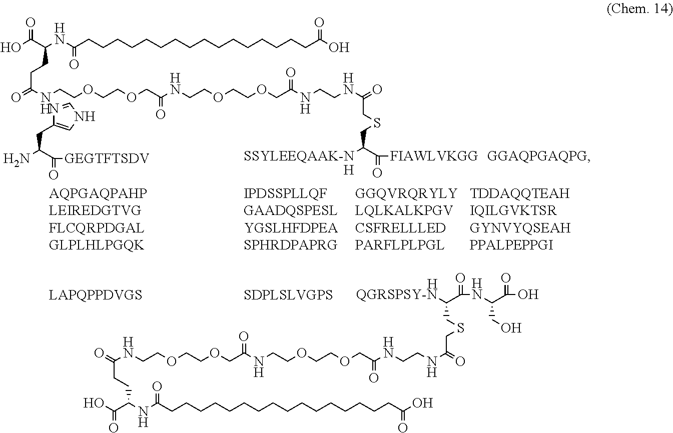

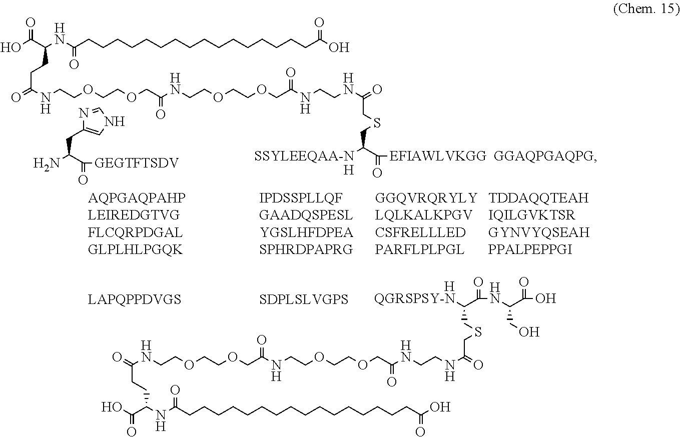

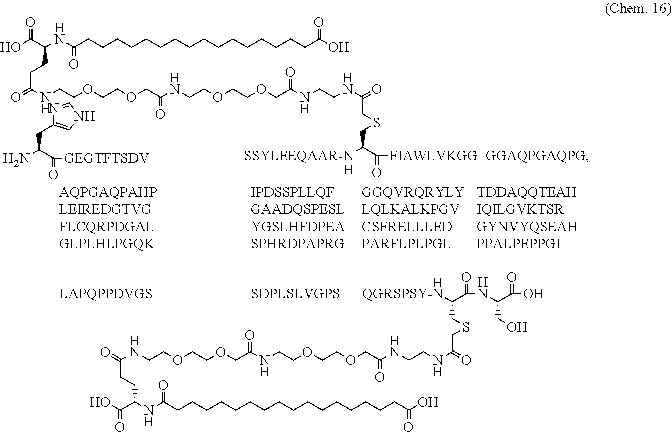

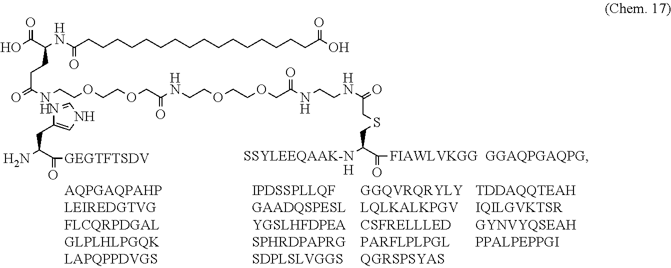

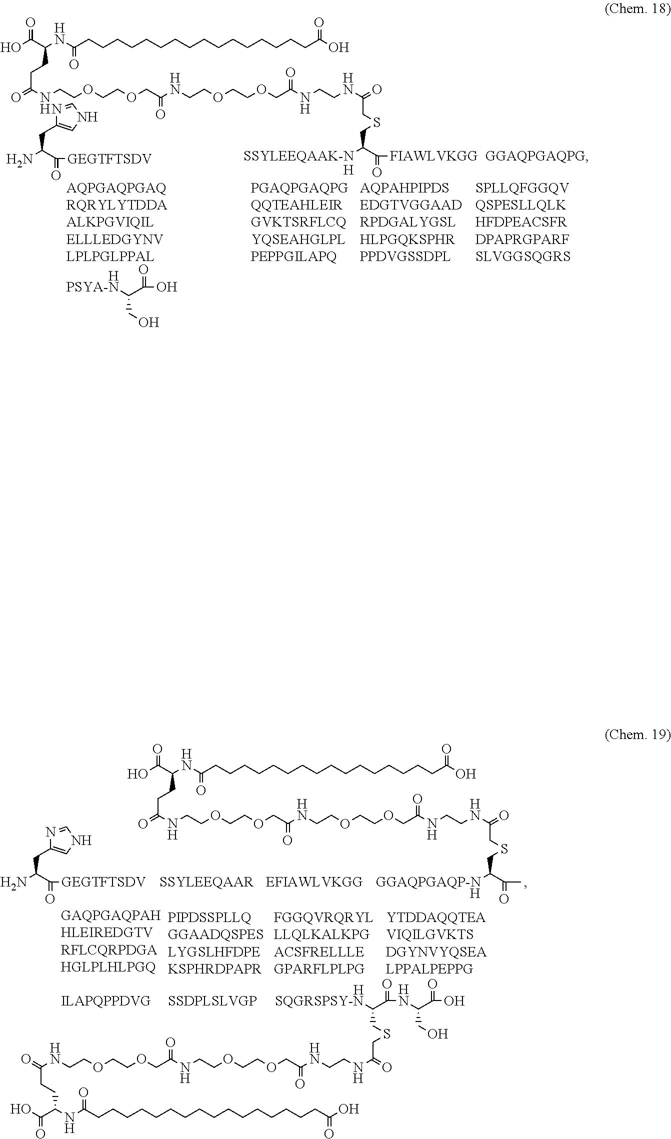

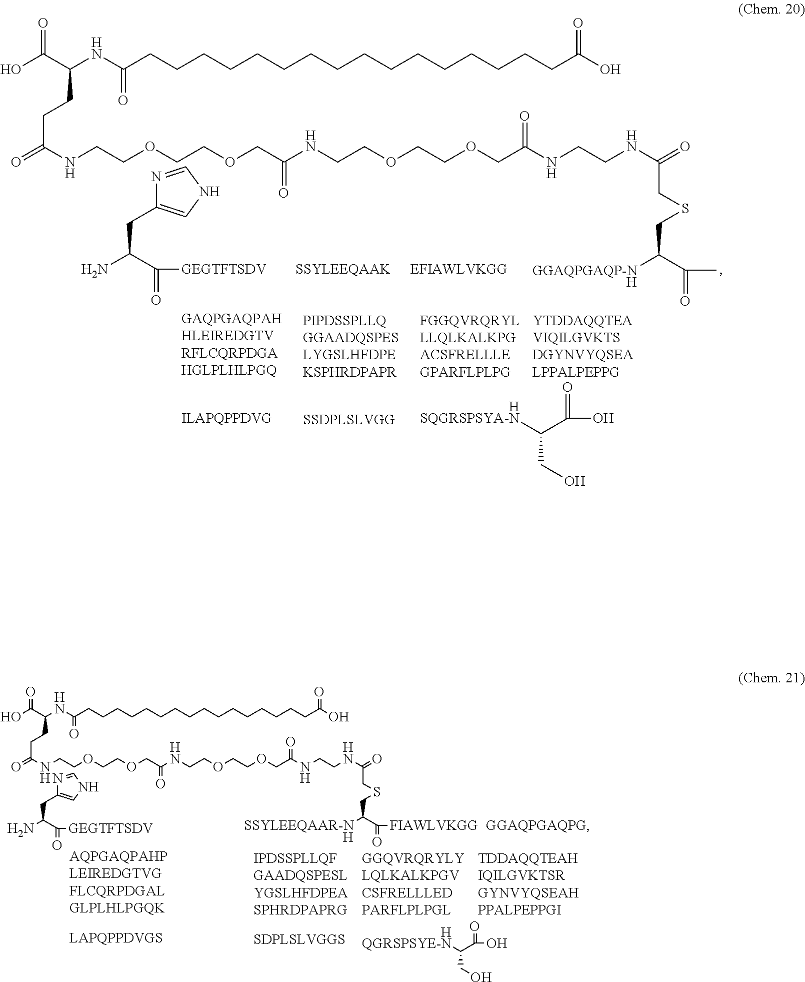

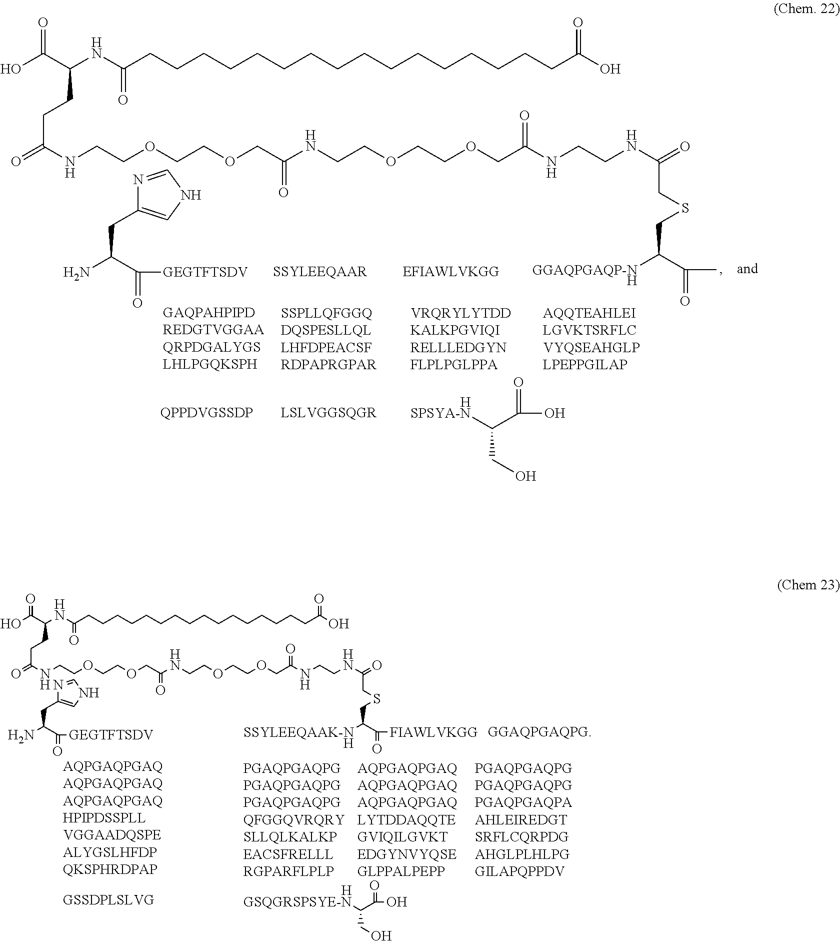

61. The fusion compound according to any one of embodiments 24-60, wherein the fusion compound is selected from a list consisting of Chem. 10, Chem. 11, Chem. 12, Chem. 14, Chem. 16, Chem. 17, Chem. 18, Chem. 21, and Chem. 23.
62. The fusion compound according to any one of embodiments 24-60, wherein the fusion compound is selected from a list consisting of

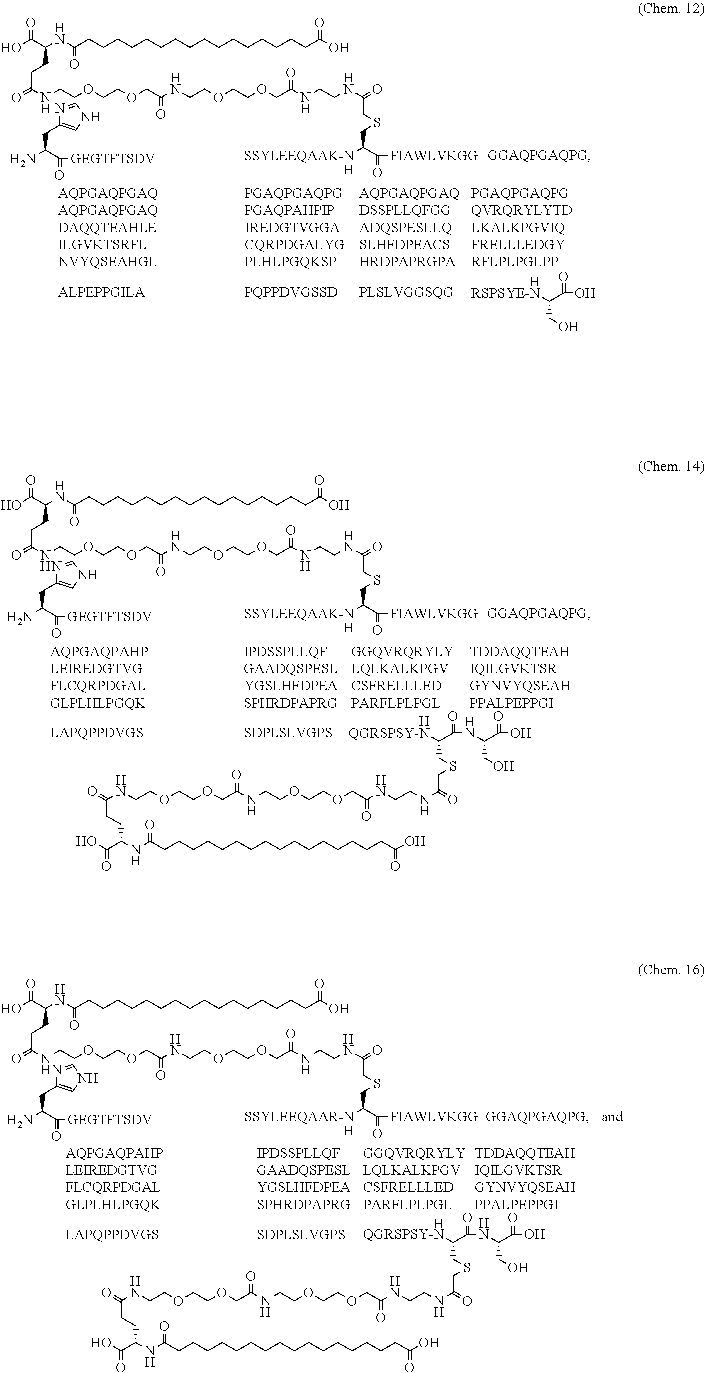

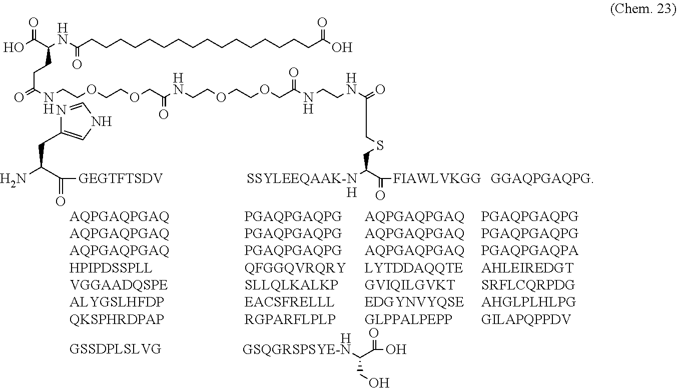

63. The fusion compound according to any one of embodiments 24-61, wherein the fusion compound is

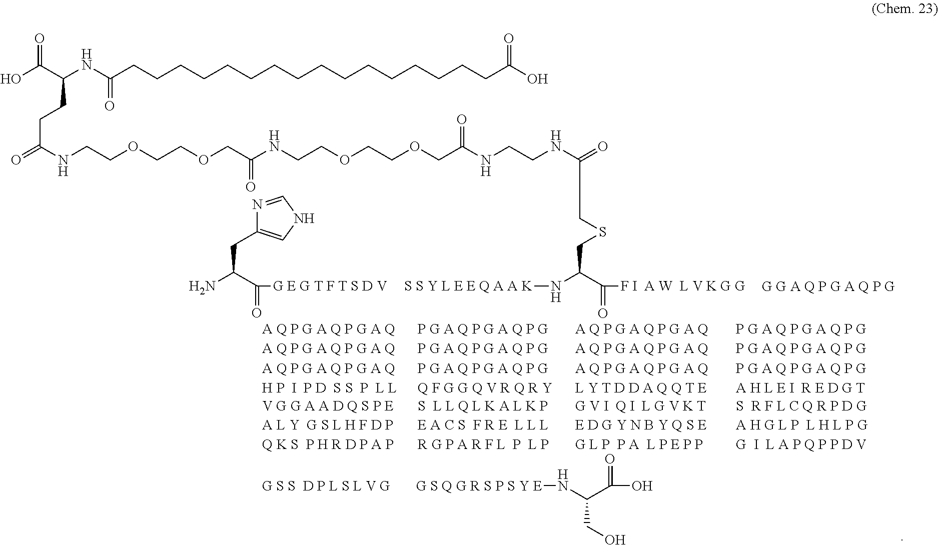

64. The fusion compound according to any one of the embodiments 24-63, wherein the fusion compound has GLP-1 activity.
65. The fusion compound according to any one of the embodiments 24-63, wherein the fusion compound is capable of binding to a GLP-1 receptor.
66. The fusion compound according to any one of the embodiments 24-63, wherein the GLP-1 activity is measured in the absence of HSA.
67. The fusion compound according to any one of the embodiments 24-66, wherein the GLP-1 activity is measured as described in Example 2.
68. The fusion compound according to any one of the embodiments 24-66, wherein the GLP-1 activity is expressed as EC50 value.
69. The fusion compound according to embodiment 70, wherein the EC50 value is measured without the presence of HSA and is below about 100 pM, preferably below about 70 μm, preferably below about 50 pM, preferably below about 20 pM.
70. The fusion compound according to any one of the embodiments 24-66, wherein the fusion compound has FGF21 activity.
71. The fusion compound according to any one of the embodiments 24-70, wherein the fusion compound is measured in the absence of HSA.
72. The fusion compound according to any one of the embodiments 24-70, wherein the fusion compound is measured as described in Example 3.
73. The fusion compound according to any one of the embodiments 24-70, wherein the FGF21 activity is the capability to activate FGFR.
74. The fusion compound according to any one of the embodiments 24-70, wherein the FGF21 activity is the capability to activate FGFR1c.
75. The fusion compound according to any one of the embodiments 24-70, wherein the FGF21 activity is measured in vitro using a whole cell assay.
76. The fusion compound according to any one of the embodiments 24-70, wherein the FGF21 activity is expressed as the EC₅₀ value, and wherein the EC₅₀ is below about 50 nM, preferably below about 10 nM, preferably below about 5 nM, preferably below about 2 nM.
77. The fusion compound according to any one of the embodiments 24-70, wherein the fusion compound has a desirable half-life.
78. The fusion compound according to any one of the embodiments 24-77, wherein the fusion compound has a long half-life.
79. The fusion compound according to any one of the embodiments 24-77, wherein the fusion compound has a half-life which is suitable for once weekly dosing.
80. The fusion compound according to any one of the embodiments 24-79, wherein the fusion compound is protracted.
81. The fusion compound according to any one of the embodiments 24-77, wherein the fusion compound is capable of lowering plasma levels of triglycerides.
82. A pharmaceutical composition comprising the fusion compound according to any one of 24-81, or a pharmaceutically acceptable salt, amide or ester thereof, and one or more pharmaceutically acceptable excipients 83. The fusion compound according to any one of embodiments 24-81, or the pharmaceutical composition according to embodiment 82, for use as a medicament.
84. The fusion compound according to any one of embodiments 24-81, or the pharmaceutical composition according to embodiment 82, for use in
(i) prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1C;
(ii) delaying or preventing diabetic disease progression, such as progression in type 2 diabetes, delaying the progression of impaired glucose tolerance (IGT) to insulin requiring type 2 diabetes, delaying or preventing insulin resistance, and/or delaying the progression of non-insulin requiring type 2 diabetes to insulin requiring type 2 diabetes;
(iii) improving β-cell function, such as decreasing β-cell apoptosis, increasing β-cell function and/or β-cell mass, and/or for restoring glucose sensitivity to β-cells;
(iv) prevention and/or treatment of cognitive disorders and/or neurodegenerative disorders, such as Alzheimer's disease, Parkinson's disease, and/or multiple sclerosis;
(v) prevention and/or treatment of eating disorders, such as obesity, e.g. by decreasing food intake, reducing body weight, suppressing appetite, inducing satiety; treating or preventing binge eating disorder, bulimia nervosa, and/or obesity induced by administration of an antipsychotic or a steroid; reduction of gastric motility; delaying gastric emptying; increasing physical mobility; and/or prevention and/or treatment of comorbidities to obesity, such as osteoarthritis and/or urine incontinence;
(vi) prevention and/or treatment of diabetic complications, such as angiopathy; neuropathy, including peripheral neuropathy; nephropathy; and/or retinopathy;
(vii) improving lipid parameters, such as prevention and/or treatment of dyslipidemia, lowering total serum lipids; increasing HDL; lowering small, dense LDL; lowering VLDL; lowering triglycerides; lowering cholesterol; lowering plasma levels of lipoprotein a (Lp(a)) in a human; inhibiting generation of apolipoprotein a (apo(a)) in vitro and/or in vivo;
(viii) prevention and/or treatment of cardiovascular diseases, such as syndrome X, atherosclerosis, myocardial infarction, coronary heart disease, reperfusion injury, stroke, cerebral ischemia, an early cardiac or early cardiovascular disease, left ventricular hypertrophy, coronary artery disease, hypertension, essential hypertension, acute hypertensive emergency, cardiomyopathy, heart insufficiency, exercise intolerance, acute and/or chronic heart failure, arrhythmia, cardiac dysrhythmia, syncopy, angina pectoris, cardiac bypass and/or stent reocclusion, intermittent claudication (atheroschlerosis oblitterens), diastolic dysfunction, and/or systolic dysfunction; and/or reduction of blood pressure, such as reduction of systolic blood pressure;
(ix) prevention and/or treatment of gastrointestinal diseases, such as inflammatory bowel disease, short bowel syndrome, or Crohn's disease or colitis; dyspepsia, and/or gastric ulcers; and/or inflammation, such as psoriasis, psoriactic arthritis, rheumatoid arthritis, and/or systemic lupus erythematosus;
(x) prevention and/or treatment of critical illness, such as treatment of a critically ill patient, a critical illness poly-nephropathy (CIPNP) patient, and/or a potential CIPNP patient; prevention of development of critical illness or CIPNP; prevention, treatment and/or cure of systemic inflammatory response syndrome (SIRS) in a patient; prevention or reduction of the likelihood of a patient suffering from bacteraemia, septicaemia, and/or septic shock during hospitalisation; and/or stabilising blood glucose, insulin balance and optionally metabolism in intensive care unit patients with acute illness; (xi) prevention and/or treatment of polycystic ovary syndrome (PCOS); (xii) prevention and/or treatment of cerebral disease, such as cerebral ischemia, cerebral haemorrhage, and/or traumatic brain injury;
(xiii) prevention and/or treatment of sleep apnoea; and/or
(xiv) prevention and/or treatment of abuse, such as alcohol abuse and/or drug abuse.
85. The fusion compound according to any one of embodiments 24-81, or the pharmaceutical composition according to embodiment 82, for use in the treatment and/or prevention any one of all forms of diabetes and related diseases, such as obesity, eating disorders, cardiovascular diseases, diabetic complications; and/or for improving lipid parameters, improving β-cell function; and/or for delaying or preventing diabetic disease progression; and/or for of treatment and/or prevention of hepatic steatosis and non-alcoholic fatty liver disease (NAFLD).
86. The fusion compound according to any one of embodiments 24-81, or the pharmaceutical composition according to embodiment 82, for use in the prevention and/or treatment of obesity.
87. The fusion compound according to any one of embodiments 24-81, or the pharmaceutical composition according to embodiment 82, for use in the treatment and/or prevention of hepatic steatosis and non-alcoholic fatty liver disease (NAFLD).
88. The fusion compound according to any one of embodiments 24-81, or the pharmaceutical composition according to embodiment 82, for use in the treatment and/or prevention of NASH.
89. The fusion compound according to any one of embodiments 24-81, or the pharmaceutical composition according to embodiment 82, for use in the prevention or delay in increase in the relative liver weight, plasma alanine aminotransferase levels, liver triglyceride content and/or liver cholesterol.
90. The fusion compound according to any one of embodiments 24-81, or the pharmaceutical composition according to embodiment 82, for use in the reduction of relative liver weight, plasma alanine aminotransferase levels, liver triglyceride content and/or liver cholesterol.
91. The fusion compound according to any one of embodiments 24-81, or the pharmaceutical composition according to embodiment 82, for use in the prevention, delay and/or reduction of inflammation in the liver.
92. The fusion compound according to any one of embodiments 24-81, or the pharmaceutical composition according to embodiment 82, for use in the prevention, delay and/or reduction of fibrogenesis in the liver.
93. Use of a fusion compound according to any one of embodiments 24-81, or the pharmaceutical composition according to embodiment 82, in the manufacture of a medicament for the treatment and/or prevention of all forms of diabetes and related diseases, such as obesity, eating disorders, cardiovascular diseases, diabetic complications; and/or for improving lipid parameters, improving β-cell function; and/or for delaying or preventing diabetic disease progression; and/or for of treatment and/or prevention of hepatic steatosis and non-alcoholic fatty liver disease (NAFLD).
94. Use of a fusion compound according to any one of embodiments 24-81, or a pharmaceutical composition according to embodiment 82, in the manufacture of a medicament for the treatment and/or prevention of NASH.
95. Use of a fusion compound according to any one of embodiments 24-81, or a pharmaceutical composition according to embodiment 82, in the manufacture of a medicament for the treatment of hepatic steatosis and non-alcoholic fatty liver disease (NAFLD).
96. A method for treating or preventing all forms of diabetes and related diseases, such as obesity, eating disorders, cardiovascular diseases, diabetic complications; and/or for improving lipid parameters, improving β-cell function; and/or for delaying or preventing diabetic disease progression; and/or for of treatment and/or prevention of hepatic steatosis and non-alcoholic fatty liver disease (NAFLD) by administering a pharmaceutically active amount of a fusion compound according to any one of embodiments 24-81, or a pharmaceutical composition according to embodiment 82.
97. A method for treating or preventing NASH by administering a pharmaceutically active amount of a fusion compound according to any one of embodiments 24-81, or a pharmaceutical composition according to embodiment 82.
98. A method of treating or preventing hepatic steatosis and non-alcoholic fatty liver disease NAFLD) by administering a pharmaceutically active amount of a compound according to any one of embodiments 24-81, or the pharmaceutical composition according to embodiment 82.
99. A method of producing the fusion compounds according to any one of embodiments 24-81, the method comprising the steps of (i) recombinant preparation of the backbone polypeptide, and (ii) covalent attachment (e.g. by alkylation) of a substituent to the backbone polypeptide.

### FURTHER PARTICULAR EMBODIMENTS

A. A fusion compound comprising a polypeptide of formula Chem. 1: A-B-C, and optionally one or more substituents, wherein:
   A is a GLP-1 polypeptide, which is an analogue of SEQ ID NO:1;
   B is a spacer consisting of 1-257 amino acids;
   C is an FGF21 polypeptide, which is an analogue of SEQ ID NO: 2;
or a pharmaceutically acceptable salt, amide or ester thereof.
B. The fusion compound according to any one of the preceding embodiments, wherein the C-terminus of the GLP-1 polypeptide is fused to the N-terminus of the peptide spacer, and the C-terminus of the peptide spacer is fused to the N-terminus of the FGF21 analogue.
C. The fusion compound according to one of the preceding embodiments, wherein B consist of 5 to 257 amino acids.
D. The fusion compound according to one of the preceding embodiments, wherein B consist of 9 to 129 amino acids.
E. The fusion compound according to one of the preceding embodiments, wherein B comprises or consists of the formula Chem. 2 (GAQP)ₓ—C_{y}-(GAQP)ₕ-Aⱼ, wherein x is an integer in the range of 0-64, y is an integer in the range of 0-1, h is an integer in the range of 1-64 and j is an integer in the range of 0-1.
F. The fusion compound according to one of the preceding embodiments, wherein B is selected from a list consisting of: SEQ ID NO: 13, SEQ ID NO: 14, SEQ ID NO: 15, SEQ ID NO: 16, SEQ ID NO: 17, and SEQ ID NO: 18.
G. The fusion protein according to one of the preceding embodiments, wherein B is SEQ ID NO: 13, SEQ ID NO: 16, or SEQ ID NO: 17.
H. The fusion compound according to one of the preceding embodiments, wherein A is at least 50%, preferably at least 55%, preferably at least 60%, preferably at least 65%, preferably at least 70%, preferably at least 75%, preferably at least 80%, preferably at least 85%, preferably at least 90%, or most preferably at least 95%, identical to SEQ ID NO: 1.
I. The fusion compound according to one of the preceding embodiments, wherein A comprises a maximum of 15 amino acid changes, preferably a maximum of 14 amino acid changes, preferably a maximum of 13 amino acid changes, preferably a maximum of 12 amino acid changes, preferably a maximum of 11 amino acid changes, preferably a maximum of 10 amino acid changes, preferably a maximum of 9 amino acid changes, preferably a maximum of 8 amino acid changes, preferably a maximum of 7 amino acid changes, preferably a maximum of 6 amino acid changes, preferably a maximum of 5 amino acid changes, and most preferably a maximum of 4 amino acid changes, as compared to SEQ ID NO: 1.
J. The fusion compound according to any preceding embodiment, wherein A comprises of consists of the amino acid sequence SEQ ID NO: 3.
K. The fusion compound according to any preceding embodiment, wherein A has 3-7 amino acid changes as compared to SEQ ID NO: 1.
L. The fusion compound according to any preceding embodiment, wherein A comprises a Cys residue at a position corresponding to position 26 or 27 or 36 of GLP-1(7-37) (SEQ ID NO:1), or optionally wherein A comprises an Arg residue at a position corresponding to position 26 of GLP-1(7-37) (SEQ ID NO:1)
M. The fusion compound according to any preceding embodiment, wherein A comprises the following amino acid changes as compared to GLP-1 (7-37) (SEQ ID NO: 1): [8G, 22E, 26R], [8G, 22E, 26C, 36G], [8G, 22E, 27C, 36G], [8G, 22E, 26R, 27C, 36G], [8G, 22E, 26R, 36C], [8G, 22E, 36G], or [8G, 22E, 26R, 36G].
N. The fusion compound according to any preceding embodiment, wherein A is selected from a list consisting of SEQ ID NO: 4, SEQ ID NO: 5, SEQ ID NO: 6, SEQ ID NO: 7, SEQ ID NO: 8, SEQ ID NO: 9, and SEQ ID NO: 35.
O. The fusion compound according to any preceding embodiment, wherein C is at least 50%, preferably at least 55%, preferably at least 60%, preferably at least 65%, preferably at least 70%, preferably at least 75%, preferably at least 80%, preferably at least 85%, preferably at least 90%, or most preferably at least 95%, identical to SEQ ID NO: 2.
P. The fusion compound according to any preceding embodiment, wherein C is comprises a maximum of 10 amino acid changes, preferably a maximum of 9 amino acid changes, preferably a maximum of 8 amino acid changes, preferably a maximum of 7 amino acid changes, preferably a maximum of 6 amino acid changes, preferably a maximum of 5 amino acid changes, preferably a maximum of 4 amino acid changes, most preferably a maximum of 3 amino acid changes, as compared to SEQ ID NO: 2.
Q. The fusion compound according to any preceding embodiment, wherein C has 3 or 4 amino acid changes as compared to SEQ ID NO: 2.
R. The fusion compound according to any preceding embodiment, wherein C comprises 180C or 180E or 180A.
S. The fusion compound according to any preceding embodiment, wherein C comprises the following amino acid changes as compared to FGF21(1-181) (SEQ ID NO: 2): [121Q, 168L, 180C] or [121Q, 168L, 171G] or [121Q, 168L, 171G, 180E].
T. The fusion compound according to any preceding embodiment, wherein C is selected from a list consisting of SEQ ID NO: 10, SEQ ID NO: 11, and SEQ ID NO: 12.
U. The fusion compound according to any preceding embodiment, wherein C has FGF21 activity.
V. The fusion compound according to any preceding embodiment, wherein C is capable of activating the FGFR1c.
W. The fusion compound according to any preceding embodiment, wherein the components of Chem. 1 are linked via amide bonds.

### List of Abbreviations

AUC Area under the curve
BEH Ethylene Bridged Hybrid
BHK Baby hamster kidney
BKL β klotho
BNP Brain natriuretic peptide
BSA Bovine seum albumin
BSPP Bis(p-sulfonatophenyl)phenylphosphine dihydrate dipotassium salt
CETP Cholesteryl ester transfer protein
CHO Chinese hamster ovary
CIP Clean in place
CMV Cytomegalovirus
CV Column volume
CVD Cardiovascular disease
DMEM Dubelocco's Modified Eagle Medium
DPP-IV Dipeptidyl peptidase-IV
DTT Dithiothreitol
E. coli Escherichia coli
EGF(A) Epidermal growth factor-like domain A
ELISA Enzyme-linked immunosorbent assay
EDTA Ethylenediaminetetraacetic acid
ERK Extracellular signal-regulated kinase
FBS Fetal bovine serum
FGF21 Fibroblast growth factor 21
FGF-R Fibroblast growth factor receptor
GLP-1 Glucagon-like peptide 1
HEPES 4-(2-hydroxyethyl)-1-piperazineethanesulfonic acid
HDLc High-density lipoprotein cholesterol
HRP horseradish peroxidase
HSA Human serum albumin
IMAC Immobilized metal affinity chromatography
IPTG Sopropyl β-d-1-thiogalactopyranoside
LB Luria-Bertani (Sambrook, J., E. F. Fritsch, and T. Maniatis. (1989). Molecular cloning: a laboratory manual, 2nd edition. Cold Spring Harbor Laboratory, Cold Spring Harbor, New York.)
LPa Lipoprotein a
LCMS Liquid chromatography mass spectrometry
LDL-C Low-density lipoprotein cholesterol
LDL-R Low-density lipoprotein receptor
LOCI Luminescence Oxygen Channeling Immunoassay
HDL-C High-density lipoprotein cholesterol
MAPK Mitogen-activated protein kinase
MQ Milli-Q
MSD Mass selective detector
MWCO Molweight cut off
NAFLD Non-alcoholic fatty liver disease
NEP Neutral endopeptidase
PBS phosphate buffered saline
PCA Polyclonal antibody
PCSK9 Proprotein Convertase Subtilisin/Kexin type 9
Pen/step Penicillin-streptomycin
SDS-PAGE Sodium dodecyl sulfate-polyacrylamide gel electrophoresis
SGLT2 Selective sodium-glucose co-transporter 2
TB Terrif broth
TFA Trifluoroacetic acid
TIC Total ion current
TOF Time of flight
Tris tris(hydroxymethyl)aminomethane or 2-amino-2-hydroxymethylpropane-1,3-diol
UPLC Ultra performance liquid chromatography
VLDL-C Very-low-density lipoprotein cholesterol

### Methods for Preparation of the Fusion Compounds

Polypeptides prepared for the fusion compounds of the invention may be carried out as common in the art.

General Method for Polypeptide Preparation in E. coli Host System

### Cloning and Expression

The fusion protein backbones with N-terminal, removable extensions, were recombinantly produced. This was done by expressing a DNA sequence encoding the amino acid sequence and sub-cloned into a pET11 d derived vector followed by transformation of a suitable host cell, i.e. E. coli BL21(DE3) or derivatives thereof. The DNA sequences were of synthetic origin and obtained from commercial providers (such as Thermofisher, Genescript).

E. coli Transformation

Transformation of E. coli was performed by standard methods according to Sambrook et al. (1989) [Sambrook J, Fritsch E F, Maniatis T.; Molecular Cloning: A Laboratory Manual, 2^{nd} edn; Cold Spring Harbor Laboratory Press: New York; 1989] or by electroporation with a Bio-Rad Gene Pulser set at 25 μF, 200 ohm, and 2.5 kV in 2-mm cuvettes according to Dower et al. (1988) [Dower, W. J., Miller, J. F., & Ragsdale, C. W. (1988) Nucleic Acids Res. 16, 6127-6145]. Transformed cells were selected on LB media supplemented with the appropriate selective antibiotics, i.e. ampicillin or kanamycin.

E. coli Cultivation

E. coli cells transformed with plasmid DNA were either taken from a frozen stock or directly from fresh transformations on LB plates (with appropriate antibiotics). Cells were inoculated into 500 ml Corning® Disposable Erlenmeyer Flask filled with 100 ml LB medium plus appropriate antibiotics. The cells were grown overnight at 30° C. with shaking at 220 rpm. 40 ml cells from the pre-culture were diluted into 2000 ml of TB medium filled into 5-L Corning® Disposable Erlenmeyer Flask. The cells were cultivated at 37° C. to OD600 2.0. Target protein expression was then induced by addition of 1 mM IPTG and further cultivated at 37° C. Expression samples were analyzed by SDS-PAGE. Inclusion body fractions were isolated and collected with sonication and centrifugation as described in the next paragraph.

### Inclusion Body Recovery

Cell slurry in 20 mM histidine, 150 mM NaCl buffer pH 6.0 was lysed with disruptor (900 bar, four passes), and pelleted by centrifugation (6000×g, for 30 minutes). The inclusion body was washed twice with 20 mM histidine (aq), 1M sodium acetate (aq), 0.1% Triton and once with water (MilliQ) and analyzed by SDS-PAGE.

### SDS-PAGE

SDS-PAGE was carried out as standard in the art using NuPAGE™ 4-12% gels Bis-Tris (Thermofisher) to analyse expression samples according to the supplied protocols.

### Refolding of Polypeptides

The inclusion bodies were solubilized in 6M urea in 20 mM ethanolamine pH 9.0 and 20 mM cysteamine. The solution was diluted into refolding buffer (20 mM Tris, pH 8.0) to the final concentration of 1 mg/ml polypeptide. The refolding process lasted for at least 12 hours at room temperature. Then insoluble impurities were removed by centrifugation (7000×g, for 45 minutes).

### Purification of Refolded Polypeptides

The solution of refolded polypeptides was loaded onto anion exchange chromatography (20 mM Tris pH 8.0, 0-500 mM NaCl) Q Sepharose Big Beads resin (GE Healthcare), as generally described in Protein Purification. Principles and Practice Series: Springer Advanced Texts in Chemistry Scopes, Robert K. 3rd ed., 1994 (Chapters 6 and 8). The N-terminal extension was removed by enterokinase cleavage to obtain the specific N-terminal of the target polypeptide. The resulting polypeptide solution was applied to Capto Phenyl highsub (GE Healthcare) (10 mM Tris pH 8.0, 1.5-0 mM NaCl) to remove enterokinase and low molecular weight impurities. The resulting pool was loaded to SOURSE30Q (GE Healthcare) (20 mM Tris pH 8.0, 0-250 mM NaC) for polishing. The final pool of target polypeptide was concentrated to 5 mg/m and stored frozen.

Capture with Capto Phenyl Highsub (GE Healthcare)

| Parameter | Information |
|---|---|
| Resin Name | Capto Phenyl highsub (GE Healthcare) |
| Resin Bed Height | 20 cm |
| Load Capacity | Aiming at <10 g/L |
| Run Temperature | Room temperature 22 ± 1° C. |
| Buffer | Buffer A: 1.5M NaCl, 20 mM Tris, pH 8.0 |
| | Buffer B: MQ H2O |
| | Buffer C: 1M NaOH (CIP) |
| Application | Conductivity adjusted to 34-34.5 mS/cm with sodium |
| treatment | chloride. pH check as 8.0 |

| | | | Flow Rate (column | Gradient |
|---|---|---|---|---|
| Program | Buffer No. | CV | volume/hour) | (%) |
| CIP | Buffer C | 3 | 6 CV/h | — |
| Wash | Buffer B | 2.5 | 10 CV/h | — |
| Equilibration | Buffer A | 3 | 10 CV/h | — |
| Sample | — | | 10 CV/h | — |
| Application | | | | |
| Column Wash | Buffer A | 4 | 10 CV/h | — |
| Elution | Buffer A + B | 3 | 10 CV/h | 99% B |

### Polishing SOURSE300 (GE Healthcare)

| Parameter | Information |
|---|---|
| Resin Name | SOURSE30Q (GE Healthcare) |
| Resin Bed Height | 20 cm |
| Load Capacity | Aiming at <10 g/L |
| Run Temperature | Room temperature 22 ± 1° C. |
| Buffer | Buffer A: 20 mM Tris, pH 8.0 |
| | Buffer B: 400M NaCl 20 mM Tris, pH 8.0 |
| | Buffer C: 1M NaOH |
| Application | Conductivity adjusted to below 4.0 mS/cm with |
| treament | Millipore water. pH check as 8.0 |

| | | | Flow Rate | |
|---|---|---|---|---|
| | | | (column | Gradient |
| Program | Buffer No. | CV | volume/hour) | (%) |
| CIP | Buffer C | 3 | 6 CV/h | — |
| Wash | Buffer B | 2.5 | 12 CV/h | — |
| Equilibration | Buffe rA | 3 | 12 CV/h | — |
| Sample Application | — | | 12 CV/h | — |
| Column Wash1 | Buffe rA | 4 | 12 CV/h | — |
| Column Wash2 | Buffer A + B | 3 | 12 CV/h | 15% B |
| Elution 1 | Buffer A + B | 10 | 12 CV/h | 15-40% B |
| | | | | over |
| | | | | 10 CV |
| Elution 2 | Buffer A + B | 3 | 12 CV/h | 40% B |

### General Method for Introduction of the Substituent on the Polypeptide and Purification of the Fusion Compound

The reagent (Chem. 7) needed to introduce the substituent on the polypeptide was prepared as described in WO2016/102562:

### 17-{(S)-1-carboxy-3-[2-(2-{[2-(2-{[2-(2-bromoacetyl-amino)ethylcarbamoyl]methoxy}ethoxy)ethylcarbamoyl]methoxy}ethoxy)ethylcarbamoyl]propylcarbamoyl}heptadecanoic acid

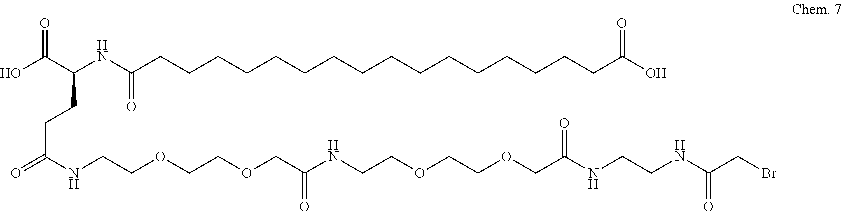 [SMILES: O=C(O)CCCCCCCCCCCCCCCCC(=O)N[C@@H](CCC(=O)NCCOCCOCC(=O)NCCOCCOCC(=O)NCCNC(=O)CBr)C(=O)O]

### Alkylation

The frozen polypeptide solution was thawed and then concentrated to above 1 mg/ml using spin filters MWCO 10 KDa (30 min, 3000 rpm). The pH was adjusted to 8.5 with aq. NaOH, and 5 eq. BSPP per capped cysteine was added. After 2-3 h of stirring, 4-5 eq. of Chem. 7 in 0.1 M NaHCO₃ (aq.) per free cysteine were added. The mixture was stirred gently in the dark for 1.5-16 hours. The reaction mixture was diluted with water before purification by anion exchange using an Äkta system:

| | |
|---|---|
| Column | Sourse 30Q, 26 ml |
| Buffer A | 20 mM Tris, 50 mM NaCl, pH = 7.8 |
| Buffer B1 | 20 mM Tris, 500 mM NaCl, pH = 7.8 |
| Buffer B2 | 20 mM Tris, 1000 mM NaCl, pH = 7.8 |
| Equilibration | 2 CV of buffer A |
| Loading flow | 5 ml/min |
| Wash | 2 CV buffer A |
| Gradient | Typically 30 CV from 10% B up to 90% buffer B |
| Flow | 5 ml/min |

The pure fractions were pooled and buffer-exchanged to either Buffer B1: 8 mM Phosphate, 240 mM Propylene Glycol, 0.007% Tween 20, pH=8.2 or Buffer B2: 10 mM Phosphate, 2% (w/vol) Glycerol, pH 8.2 using an Akta system:

| | |
|---|---|
| Column | HiPrep 26/10, 53 ml |
| Buffer | B1: 8 mM Phosphate, 240 mM Propylene Glycol, |
| | 0.007% Tween 20, pH = 8.2 |
| | B2: 10 mM Phosphate, 2% (w/vol) Glycerol, pH 8.2 |
| Equilibration | 2 CV |
| Loading flow | 10 ml/min |
| Flow | 10 ml/min |
| Gradient | 1.5 CV, Isocratic |

If needed, the pooled fractions were concentrated to 3-5 mg/ml using spin filters (30 min, 3000 rpm).

### General Method for Detection and Characterisation

Sample is diluted to approx. 1 mg/ml and injected to a LC-MS system ( ), e.g. 1 μl. The analogues are desalted. The instrument should be calibrated and if possible by use of lock mass spray. MS spectrum over main chromatographic peak is generated and the intact mass is reconstructed using a deconvolution algorithm.

### LC-MS Method 1 (LCMS 29)

| | |
|---|---|
| System | System: Agilent 1290 infinity series UPLC |
| | Column: Phenomenex Aeris widepore 3.6μ C4 50 × |
| | 2.1 mm |
| | Detector: Agilent Technologies LC/MSD TOF |
| | 6230 (G6230A) |
| Detector setup | Ionisation method: Agilent Jet Stream source |
| | Scanning range: m/z min. 100, m/z max. 3200 |
| | Linear reflector mode |
| | Positive mode |
| Conditions: | Step gradient: 5% to 90% B |
| | Gradient run-time: 10 minutes: 0-1 min 5-20% B, |
| | 1-7 min 20-90% B, 7-8 min 90% B, 8-8.5 min 90-5% |
| | B, 8.5-10 min 5% B |
| | Flow rate: 0.40 ml/min fixed |
| | Column temperature: 40° C. |
| Eluents | Solvent A: 99.98% H₂O, 0.02% TFA |
| | Solvent B: 99.98% CH₃CN, 0.02% TFA |
| Results | Mass found was either m/z ((m + z)/z) of the compound |
| specification | for compounds with m < 4000 or mass (average) as the |
| and validation | result of a deconvolution using Masshunter Workstation |
| | Software Version B.05.00 Build 5.0.519.13 SP1 |
| | (Agilent) |
| | Calculated Mass is the average molecular weight of the |
| | desired compound |
| | Calculated m/z is the molecular weight (m + z)/z of |
| | the desired compound |

### LC-MS Method 2 (LCMS 36)

| | |
|---|---|
| System | LC-system: Waters Acquity UPLC H Class |
| | Column: Phenomenex Aeris, C-4, 3.6 μm widepore, 2.1 mm × 50 mm |
| | Detector: Waters Xevo G2-XS QTof |
| Detector setup | Ionisation method: ES Scanning range: 50-4000 amu |
| | Operating mode: MS resolution mode positive/ne: positive mode |
| | Voltage: Capillary 3.00 kV |
| | Sample cone 40 V |
| | Source 80 V; |
| | Temperature: Source 150° C. |
| | Desolvation 500° C., |
| | Scantime: 0.500 s |
| | Interscan delay: 0.014 s |
| Conditions: | Step gradient: 5% to 95% B |
| | Gradient run time: 7.5 min; total run time: 8 min: A 90-70% 1 min, |
| | 70-30% 6 min, 30-0% 0.5 min, 0% 0.5 min; B 5-25% 1 min, 25-65% 6 |
| | min, 65-95% 0.5 min, 95% 0.5 min; C 5% 8 min |
| | Flow rate: 0.4 ml/min |
| | Column temperature: 40° C. |
| Eluents | Solvent A: 99.90% MQ-water, 0.1% formic acid |
| | Solvent B: 99.90% acetonitrile, 0.1% formic acid |
| | Solvent C: 99.90% MQ water 0.1% TFA |
| Results | Results specification and validation: Mass found was the mass found |
| specification | of the compound. M/z found was the molecular ion found ((M + z)/z) of |
| and validation | the compound |
| | Calculated Mass is the molecular weight of the desired compound. |
| | Calculated M/z is the molecular weight (M + z)/z of the desired |
| | compound |
| | Purity: Total ion current (TIC) AUC of analyte peak, in percent of total |
| | AUC excl solvent peak, as reported by system software. |
| | Identity: Mass of each analyte mass peak expressed as m/z from |
| | highest to lowest. |

### LC-MS Method 3 (LCMS 47)

| | |
|---|---|
| System | Waters Acquity UPLC H Class |
| | Column: Waters Aquity UPLC Protein BEH, C4, 300 Å, 1.7 um, 2.1 mm × |
| | 50 mm |
| | Detector: Waters Xevo G2-XS QTof |
| Detector setup | Ionisation method: ES Scanning range: 50-4000 amu |
| | Operating mode: MS resolution mode positive/ne: positive mode |
| | Voltage: Capillary 3.00 kV |
| | Sample cone 40 V |
| | Source 80 V |
| | Temperature: Source 150° C. |
| | Desolvation 500° C. |
| | Scantime: 0.500 s |
| | Interscan delay: 0.014 s |
| | Cone gas: 100 L/h |
| | Desolvation gas: 600 L/h |
| Conditions: | Linear gradient in steps: 5% to 95% B |
| | Gradient run-time: 8.5 minutes Total run-time: 9.0 minutes Gradient: |
| | A 90-70% 1 min 70-30% 7 min, 30-0% 0.5 min 0% 0.5 min; B 5-25% |
| | 1 min 25-65% 7 min 65-95% 0.5 min 95%; 0.5 min C 5% |
| | Flow rate: 0.4 ml/min |
| | Column temperature: 60° C. |
| Eluents | Eluents: Solvent A: MQ-water Solvent B: Acetonitrile (Optima) Solvent |
| | C: 0.1% TFA, 2% formic acid in MQ water |
| Results | Mass found was the mass found of the compound. M/z found was the |
| specification | molecular ion found ((M + z)/z) of the compound |
| and validation | Calculated Mass is the molecular weight of the desired compound |
| | Calculated M/z is the molecular weight (M + z)/z of the desired |
| | compound |
| | Purity: Total ion current (TIC) AUC of analyte peak, in percent of total |
| | AUC excluding solvent peak, as reported by system software. |
| | Identity: Mass of each analyte mass peak expressed as m/z from |
| | highest to lowest. |

### Examples 1.1-1.17

TABLE 5
Fusion compounds Chem. 8-Chem. 23

| | SEQ ID NO of the | |
|---|---|---|
| Compound: | polypeptideback | |
| Chem X | bone | Compound name |
| Chem. 8 | 19 | [8G,22E,26R]GLP-1(7-37)-(GAQP)x4,A-S{Beta-180}- |
| | | [2-[2-[[2-[2-[2-[[2-[2-[2-[[(4S)-4-carboxy-4-(17-carboxyheptadecanoyl- |
| | | amino)butanoyl]amino]ethoxy]ethoxy]acetyl]amino]ethoxy]ethoxy]acetyl]amino]- |
| | | ethylamino]-2-oxoethyl]-[121Q,168L,180C]FGF21(1-181) |
| Chem. 9 | 20 | S{Beta-26}-[2-[2-[[2-[2-[2-[[2-[2-[2-[[(4S)-4-carboxy-4-(17- |
| | | carboxyheptadecanoyl- |
| | | amino)butanoyl]amino]ethoxy]ethoxy]acetyl]amino]ethoxy]ethoxy]acetyl]amino]- |
| | | ethylamino]-2-oxoethyl]-[8G,22E,26C,36G]GLP-1(7-37)-(GAQP)x4, A- |
| | | [121Q,168L,171G]FGF21(1-181) |
| Chem. 10 | 22 | S{Beta-27}-[2-[2-[[2-[2-[2-[[2-[2-[2-[[(4S)-4-carboxy-4-(17- |
| | | carboxyheptadecanoyl- |
| | | amino)butanoyl]amino]ethoxy]ethoxy]acetyl]amino]ethoxy]ethoxy]acetyl]amino]- |
| | | ethylamino]-2-oxoethyl]-[8G,22E,27C,36G]GLP-1(7-37)-(GAQP)x4,A- |
| | | [121Q,168L,171G,180E]FGF21(1-181) |
| Chem. 11 | 25 | S{Beta-27}-[2-[2-[[2-[2-[2-[[2-[2-[2-[[(4S)-4-carboxy-4-(17- |
| | | carboxyheptadecanoyl- |
| | | amino)butanoyl]amino]ethoxy]ethoxy]acetyl]amino]ethoxy]ethoxy]acetyl]amino]- |
| | | ethylamino]-2-oxoethyl]-[8G,22E,27C,36G]GLP-1(7-37)-(GAQP)x8,A- |
| | | [121Q,168L,171G,180E]FGF21(1-181) |
| Chem. 12 | 26 | S{Beta-27}-[2-[2-[[2-[2-[2-[[2-[2-[2-[[(4S)-4-carboxy-4-(17- |
| | | carboxyheptadecanoyl- |
| | | amino)butanoyl]amino]ethoxy]ethoxy]acetyl]amino]ethoxy]ethoxy]acetyl]amino]- |
| | | ethylamino]-2-oxoethyl]-[8G,22E,27C,36G]GLP-1(7-37)-(GAQP)x16,A- |
| | | [121Q,168L,171G,180E]FGF21(1-181) |
| Chem. 13 | 28 | S{Beta-36}-[2-[2-[[2-[2-[2-[[2-[2-[2-[[(4S)-4-carboxy-4-(17- |
| | | carboxyheptadecanoyl- |
| | | amino)butanoyl]amino]ethoxy]ethoxy]acetyl]amino]ethoxy]ethoxy]acetyl]amino]- |
| | | ethylamino]-2-oxoethyl]-[8G,22E,26R,36C]GLP-1(7-37)-(GAQP)x4,A- |
| | | [121Q,168L,171G,180E]FGF21(1-181) |
| Chem. 14 | 32 | S{Beta-27}-[2-[2-[[2-[2-[2-[[2-[2-[2-[[(4S)-4-carboxy-4-(17- |
| | | carboxyheptadecanoyl- |
| | | amino)butanoyl]amino]ethoxy]ethoxy]acetyl]amino]ethoxy]ethoxy]acetyl]amino]- |
| | | ethylamino]-2-oxoethyl]-[8G,22E,27C,36G]GLP-1(7-37)-(GAQP)x4,A- |
| | | S{Beta-180}-[2-[2-[[2-[2-[2-[[2-[2-[2-[[(4S)-4-carboxy-4-(17- |
| | | carboxyheptadecanoyl- |
| | | amino)butanoyl]amino]ethoxy]ethoxy]acetyl]amino]ethoxy]ethoxy]acetyl]amino]- |
| | | ethylamino]-2-oxoethyl]-[121Q,168L,180C]FGF21 |
| Chem. 15 | 31 | S{Beta-26}-[2-[2-[[2-[2-[2-[[2-[2-[2-[[(4S)-4-carboxy-4-(17- |
| | | carboxyheptadecanoyl- |
| | | amino)butanoyl]amino]ethoxy]ethoxy]acetyl]amino]ethoxy]ethoxy]acetyl]amino]- |
| | | ethylamino]-2-oxoethyl]-[8G,22E,26C,36G]GLP-1(7-37)-(GAQP)x4,A- |
| | | S{Beta-180}-[2-[2-[[2-[2-[2-[[2-[2-[2-[[(4S)-4-carboxy-4-(17- |
| | | carboxyheptadecanoyl- |
| | | amino)butanoyl]amino]ethoxy]ethoxy]acetyl]amino]ethoxy]ethoxy]acetyl]amino]- |
| | | ethylamino]-2-oxoethyl]-[121Q,168L,180C]FGF21 |
| Chem. 16 | 33 | S{Beta-27}-[2-[2-[[2-[2-[2-[[2-[2-[2-[[(4S)-4-carboxy-4-(17- |
| | | carboxyheptadecanoyl- |
| | | amino)butanoyl]amino]ethoxy]ethoxy]acetyl]amino]ethoxy]ethoxy]acetyl]amino]- |
| | | ethylamino]-2-oxoethyl]-[8G,22E,26R,27C,36G]GLP-1(7-37)-(GAQP)x4,A- |
| | | S{Beta-180}-[2-[2-[[2-[2-[2-[[2-[2-[2-[[(4S)-4-carboxy-4-(17- |
| | | carboxyheptadecanoyl- |
| | | amino)butanoyl]amino]ethoxy]ethoxy]acetyl]amino]ethoxy]ethoxy]acetyl]amino]- |
| | | ethylamino]-2-oxoethyl]-[121Q,168L,180C]FGF21 |
| Chem. 17 | 21 | S{Beta-27}-[2-[2-[[2-[2-[2-[[2-[2-[2-[[(4S)-4-carboxy-4-(17- |
| | | carboxyheptadecanoyl- |
| | | amino)butanoyl]amino]ethoxy]ethoxy]acetyl]amino]ethoxy]ethoxy]acetyl]amino]- |
| | | ethylamino]-2-oxoethyl]-[8G,22E,27C,36G]GLP-1(7-37)-(GAQP)x4,A- |
| | | [121Q,168L,171G]FGF21(1-181) |
| Chem. 18 | 24 | S{Beta-27}-[2-[2-[[2-[2-[2-[[2-[2-[2-[[(4S)-4-carboxy-4-(17- |
| | | carboxyheptadecanoyl- |
| | | amino)butanoyl]amino]ethoxy]ethoxy]acetyl]amino]ethoxy]ethoxy]acetyl]amino]- |
| | | ethylamino]-2-oxoethyl]-[8G,22E,27C,36G]GLP-1(7-37)-(GAQP)x8,A- |
| | | [121Q,168L,171G]FGF21(1-181) |
| Chem. 19 | 34 | 8G,22E,26R,36G]GLP-1(7-37)-(GAQP)x2,S{Beta}-[2- |
| | | [2-[[2-[2-[2-[[2-[2-[2-[[(4S)-4-carboxy-4-(17-carboxyheptadecanoyl- |
| | | amino)butanoyl]amino]ethoxy]ethoxy]acetyl]amino]ethoxy]ethoxy]acetyl]amino]- |
| | | ethylamino]-2-oxoethyl]C,GAQPx2,A-S{Beta-180}-[2-[2-[[2-[2-[2-[[2-[2-[2- |
| | | [[(4S)-4-carboxy-4-(17-carboxyheptadecanoyl- |
| | | amino)butanoyl]amino]ethoxy]ethoxy]acetyl]amino]ethoxy]ethoxy]acetyl]amino]- |
| | | ethylamino]-2-oxoethyl]-[121Q,168L,180C]FGF21 |
| Chem. 20 | 29 | 8G,22E,36G]GLP-1(7-37)-(GAQP)x2,S{Beta}-[2-[2-[[2- |
| | | [2-[2-[[2-[2-[2-[[(4S)-4-carboxy-4-(17-carboxyheptadecanoyl- |
| | | amino)butanoyl]amino]ethoxy]ethoxy]acetyl]amino]ethoxy]ethoxy]acetyl]amino]- |
| | | ethylamino]-2-oxoethyl]C,GAQPx2,A[121Q,168L,171G]FGF21 |
| Chem. 21 | 23 | S{Beta-27}-[2-[2-[[2-[2-[2-[[2-[2-[2-[[(4S)-4-carboxy-4-(17- |
| | | carboxyheptadecanoyl- |
| | | amino)butanoyl]amino]ethoxy]ethoxy]acetyl]amino]ethoxy]ethoxy]acetyl]amino]- |
| | | ethylamino]-2-oxoethyl]-[8G,22E,26R,27C,36G]GLP-1(7-37)-(GAQP)x4,A- |
| | | [121Q,168L,171G,180E]FGF21(1-181) |
| Chem. 22 | 30 | 8G,22E,26R,36G]GLP-1(7-37)-(GAQP)x2,S{Beta}-[2- |
| | | [2-[[2-[2-[2-[[2-[2-[2-[[(4S)-4-carboxy-4-(17-carboxyheptadecanoyl- |
| | | amino)butanoyl]amino]ethoxy]ethoxy]acetyl]amino]ethoxy]ethoxy]acetyl]amino]- |
| | | ethylamino]-2-oxoethyl]C,GAQPA,[121Q,168L,171G]FGF21 |
| Chem. 23 | 27 | {S27-[(28S)-(28,46-Dicarboxy-2,7,16,25,30-pentaoxo-9,12,18,21-tetraoxa- |
| | | 3,6,15,24,29-pentaazahexatetracontan-1-yl)]-[Gly8,36,Glu22,Cys27]- |
| | | GLP-1(human)-(7-37)-peptidyl}-(Gly-Ala-Gln-prolinyl)32- |
| | | Ala-[Gln121,Leu168,Gly171,Glu180]-FGF21(human)-(1-181)-peptide |

### Example 1.1

### [8G, 22E, 26R]GLP-1(7-37)-(GAQP)x4, A-S{Beta-180}-[2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl]-[121Q, 168L, 180C]FGF21(1-181)

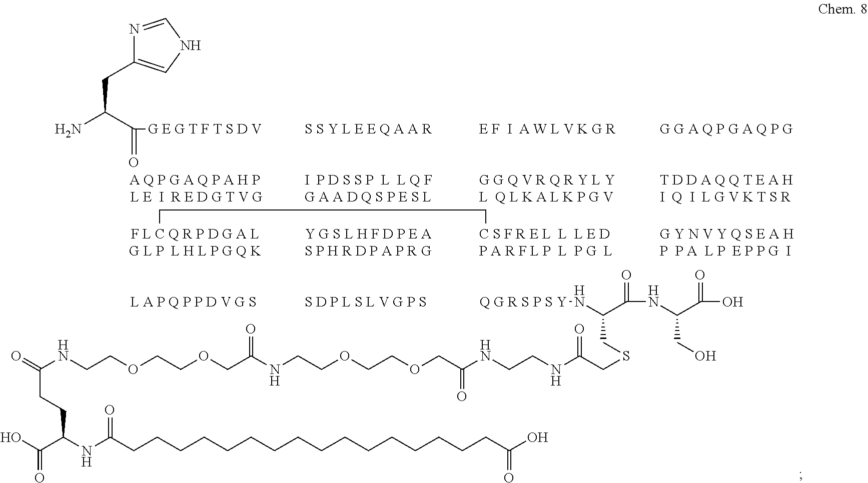

Purification using buffer B1. LCMS method 1: Calc. mass: 25161.1; Found mass: 25161.9.

### Example 1.2

### S{Beta-26}-[2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl]-[8G, 22E, 26C, 36G]GLP-1(7-37)-(GAQP)x4, A-[121Q, 168L, 171G]FGF21(1-181)

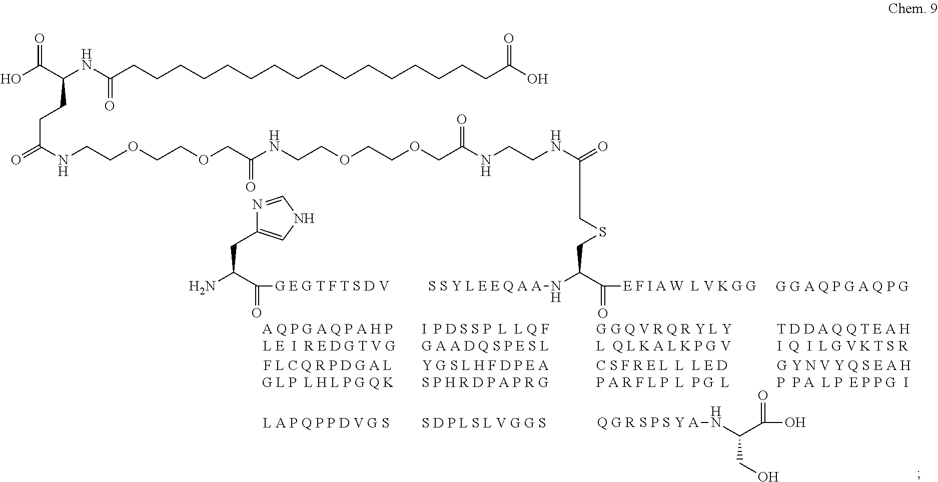

Purification using buffer B1. LCMS36: Calc. mass: 24936.6; Found mass: 24936.0

### Example 1.3

### S{Beta-27}-[2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl]-[8G, 22E, 27C, 36G]GLP-1(7-37)-(GAQP)x4, A-[121Q, 168L, 171G, 180E]FGF21(1-181)

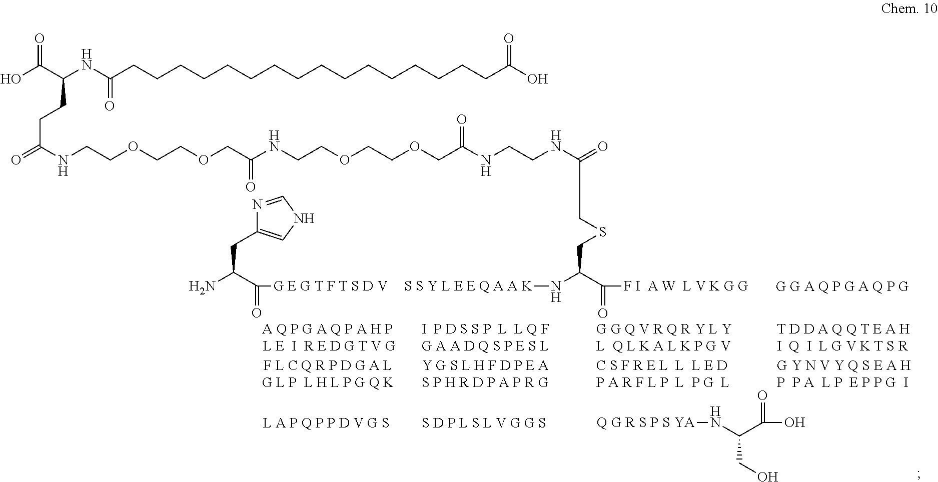

Purification using buffer B1. LCMS47: Calc. mass: 24993.7; Found mass: 24994.0

### Example 1.4

### S{Beta-27}-[2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl]-[8G, 22E, 27C, 36G]GLP-1(7-37)-(GAQP)x8, A-[121Q, 168L, 171G, 180E]FGF21(1-181)

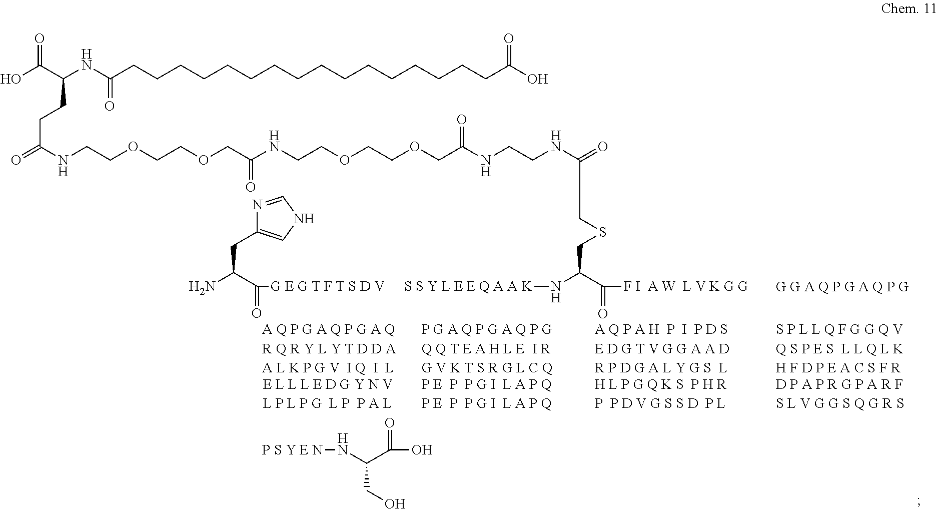

Purification using buffer B1. LCMS47: Calc. mass: 26407.2; Found mass: 26408.0

### Example 1.5

### S{Beta-27}-[2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl]-[8G, 22E, 27C, 36G]GLP-1(7-37)-(GAQP)x16, A-[121Q, 168L, 171G, 180E]FGF21(1-181)

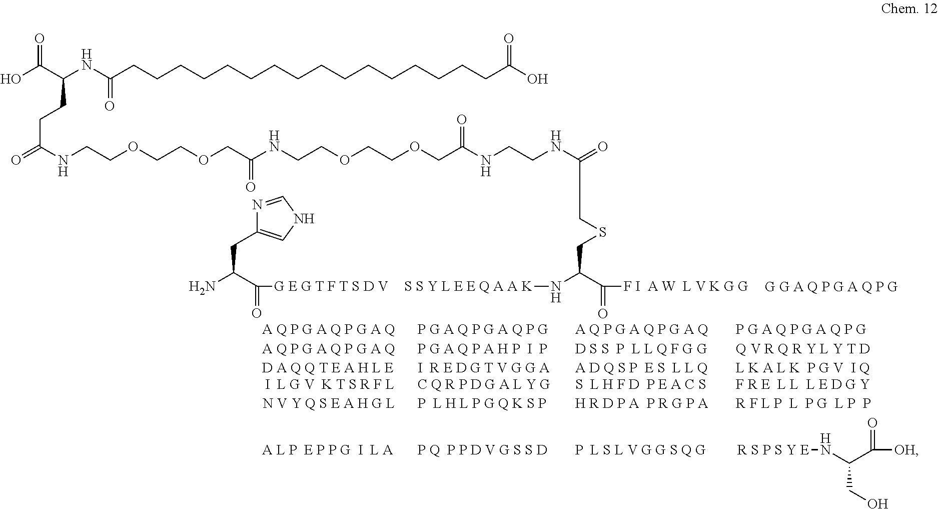

Purification using buffer B1 or B2. LCMS36: Calc. mass: 29234.2; Found mass: 29234.0

### Example 1.6

### S{Beta-36}-[2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl]-[8G, 22E, 26R, 36C]GLP-1(7-37)-(GAQP)x4, A-[121Q, 168L, 171G, 180E]FGF21 (1-181)

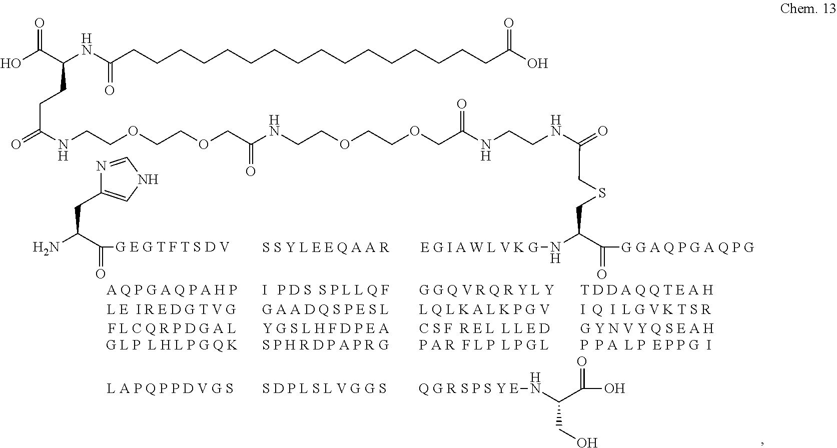

Purification using buffer B1. LCMS36: Calc. mass: 25093.8; Found mass: 25094.0

### Example 1.7

### S{Beta-27}-[2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl]-[8G, 22E, 27C, 36G]GLP-1(7-37)-(GAQP)x4, A-S{Beta-180}-[2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl]-[121Q, 168L, 180C]FGF21

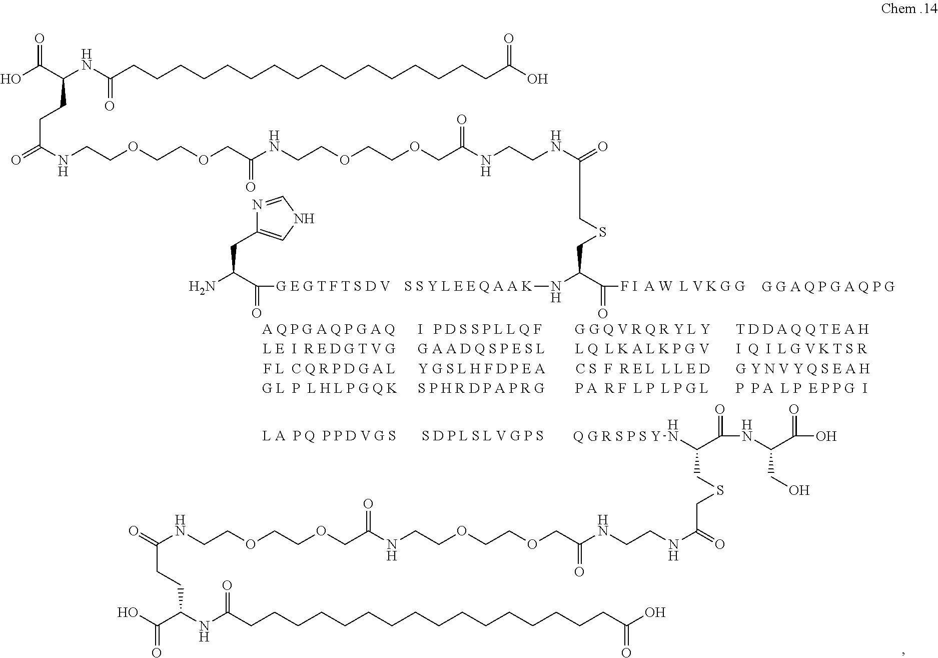

Purification using buffer B1 or B2. LCMS47: Calc. mass: 25823.8; Found mass: 25824.0

### Example 1.8

### S{Beta-26}-[2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl]-[8G, 22E, 26C, 36G]GLP-1(7-37)-(GAQP)x4, A-S{Beta-180}-[2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl]-[121Q, 168L, 180C]FGF21

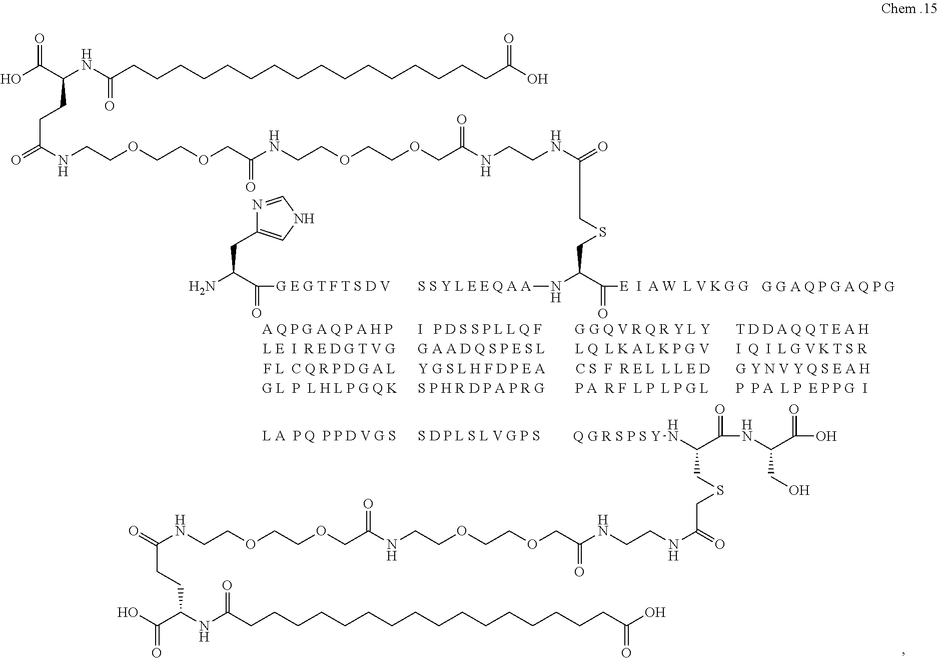

Purification using buffer B2. LCMS47: Calc. mass: 25824.7; Found mass: 25826.0

### Example 1.9

### S{Beta-27}-[2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl]-[8G, 22E, 26R, 27C, 36G]GLP-1(7-37)-(GAQP)x4, A-S{Beta-180}-[2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl]-[121Q, 168L, 180C]FGF21

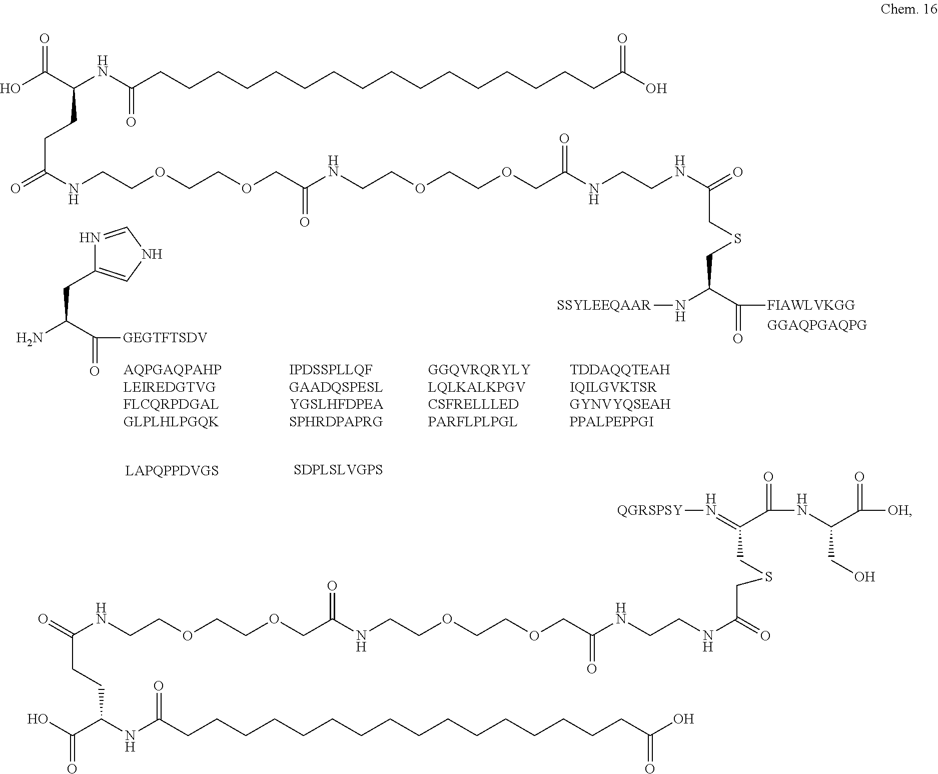

Purification using buffer B1 or B2. LCMS47: Calc. mass: 25851.8; Found mass: 25854.0

### Example 1.10

### S{Beta-27}-[2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl]-[8G, 22E, 27C, 36G]GLP-1(7-37)-(GAQP)x4, A-[121Q, 168L, 171G]FGF21(1-181)

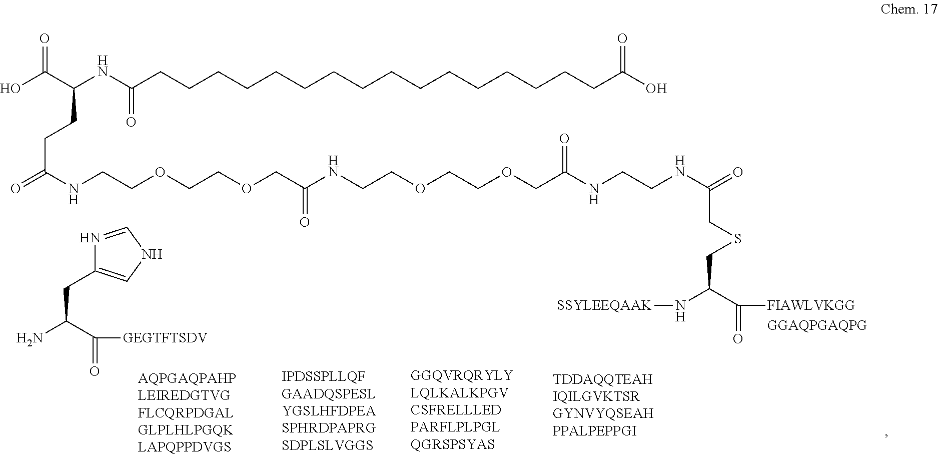

Purification using buffer B1. LCMS47: Calc. mass: 24935.7; Found mass: 24937.0

### Example 1.11

### S{Beta-27}-[2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl]-[8G, 22E, 27C, 36G]GLP-1(7-37)-(GAQP)x8, A-[121Q, 168L, 171G]FGF21(1-181)

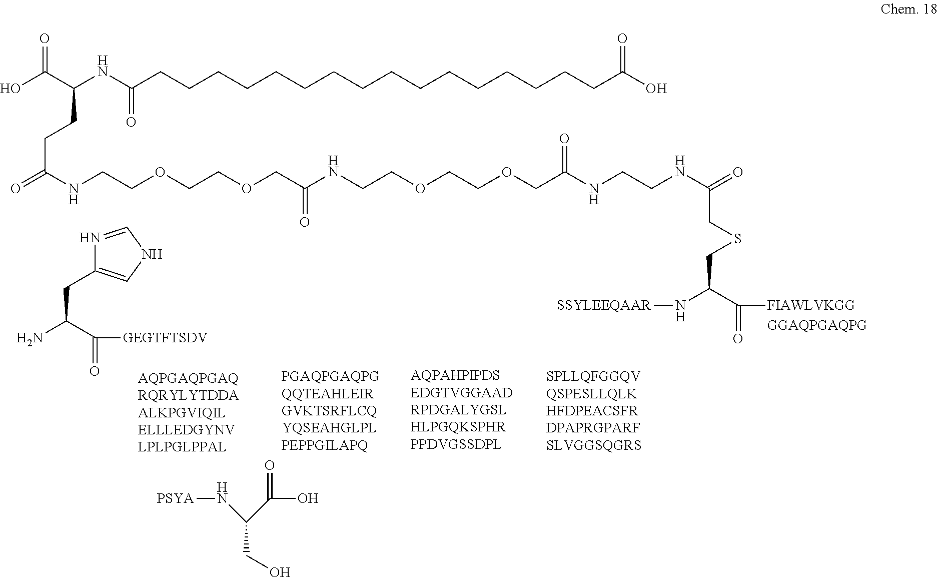

Purification using buffer B1. LCMS47: Calc. mass: 26351.2; Found mass: 26351.0

### Example 1.12

### [8G, 22E, 26R, 36G]GLP-1(7-37)-(GAQP)x2, S{Beta}-[2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl]C, GAQPx2, A-S{Beta-180}-[2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl]-[121Q, 168L, 180C]FGF21

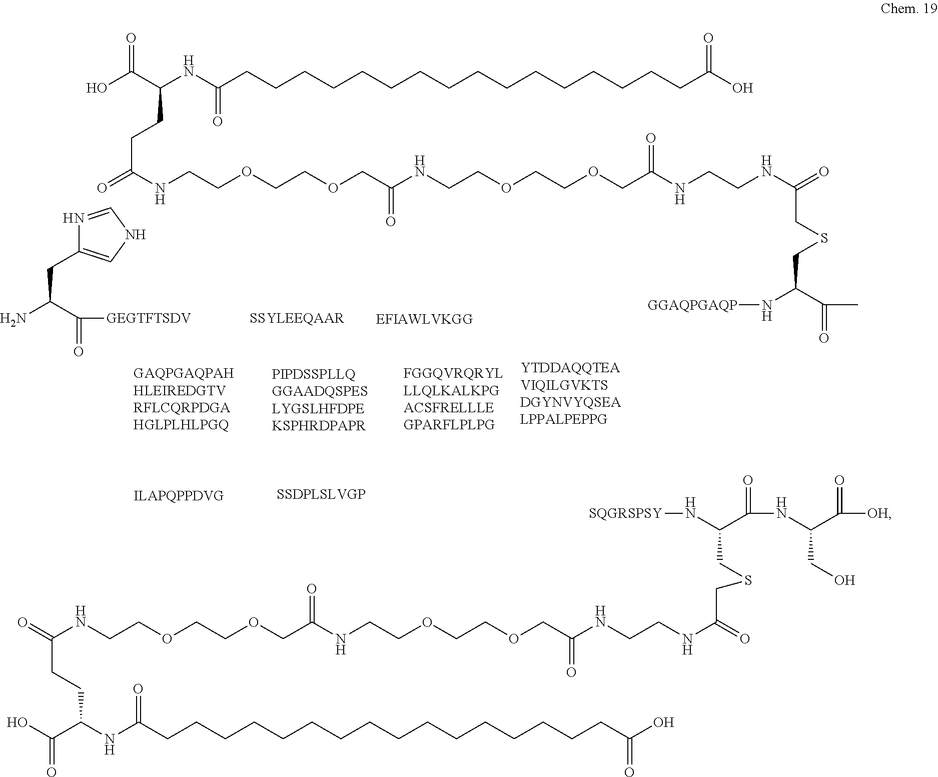

Purification using buffer B1 or B2. LCMS47: Calc. mass: 25980.9; Found mass: 25982.0

### Example 1.13

### [8G, 22E, 36G]GLP-1(7-37)-(GAQP)x2, S{Beta}-[2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl]C, GAQPx2,A [121Q, 168L, 171G]FGF21

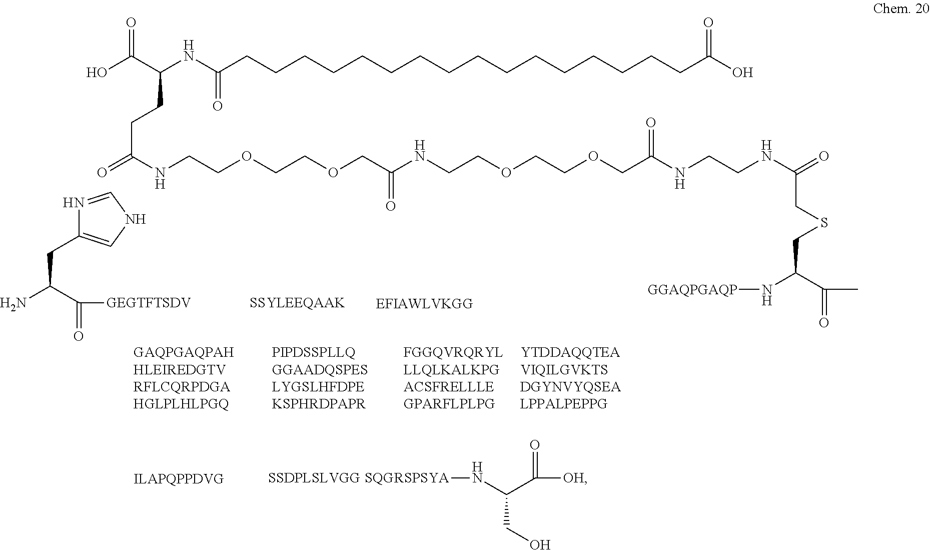

Purification using buffer B1. LCMS47: Calc. mass: 25064.8; Found mass: 25067.0

### Example 1.14

### S{Beta-27}-[2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl]-[8G, 22E, 26R, 27C, 36G]GLP-1(7-37)-(GAQP)x4, A-[121Q, 168L, 171G, 180E]FGF21(1-181)

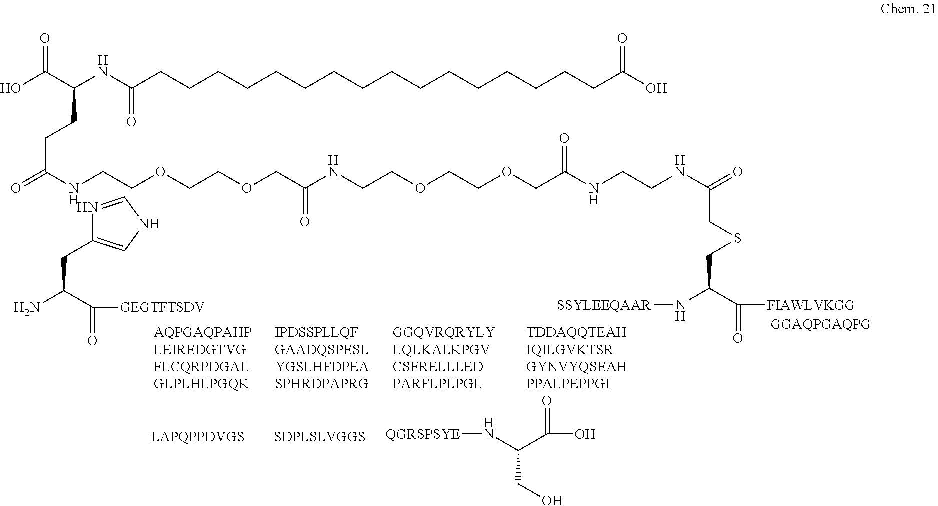

Purification using buffer B1. LCMS36: Calc. mass: 25021.7; Found mass: 25023.0

### Example 1.15

### [8G, 22E, 26R, 36G]GLP-1(7-37)-(GAQP)x2, S{Beta}-[2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl]C, GAQPA, [121Q, 168L, 171G]FGF21

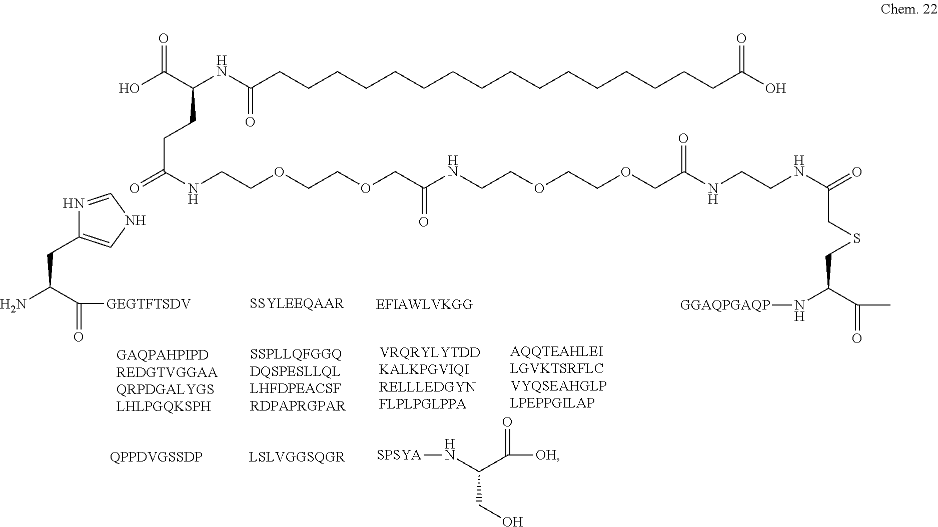

Purification using buffer B1. LCMS47: Calc. mass: 24739.4; Found mass: 24741.0

### Example 1.16

### {S27-[(28S)-(28,46-Dicarboxy-2,7,16,25,30-pentaoxo-9,12,18,21-tetraoxa-3,6,15,24,29-pentaazahexatetracontan-1-yl)]-[Gly8,36,Glu22,Cys27]-GLP-1(human)-(7-37)-peptidyl}-(Gly-Ala-Gln-prolinyl)32-Ala-[Gln121,Leu168,Gly171,Glu180]-FGF21(human)-(1-181)-peptide

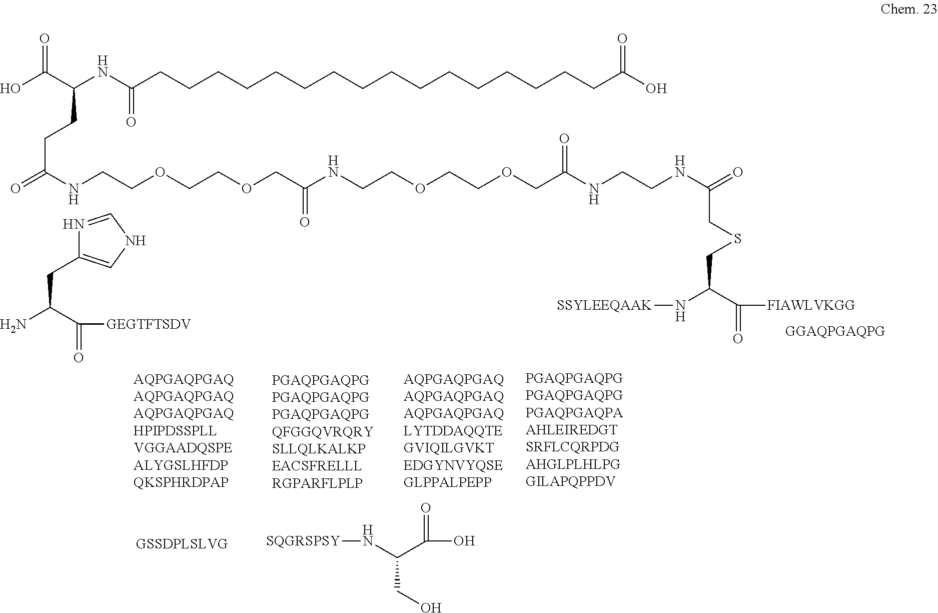

Purification using buffer B2. LCMS47: Calc. mass: 34888.2; Found mass: 34888.0

### Example 1.17—Reference Compound 1

### N-epsilon26-[2-(2-[2-(2-[2-(2-[4-(17-Carboxyheptadecanoylamino)-4(S)-carboxybutyrylamino]ethoxy)ethoxy]acetylamino)ethoxy]ethoxy)acetyl][Aib8,Arg34]GLP-1-(7-37)peptide

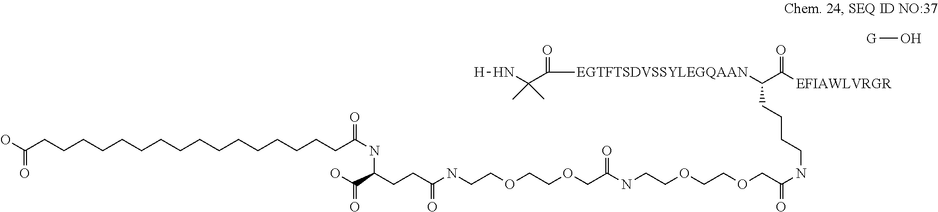

The compound was prepared as described in WO06097537.

### Example 1.18—Reference Compound 2

### S{Beta-180}-[2-[2-[[2-[2-[2-[[2-[2-[2-[[(4S)-4-carboxy-4-(17-carboxyheptadecanoyl-amino)butanoyl]amino]ethoxy]ethoxy]acetyl]amino]ethoxy]ethoxy]acetyl]amino]-ethylamino]-2-oxoethyl]-Ala[Gln121,Leu168,Cys180]FGF21(1-181)

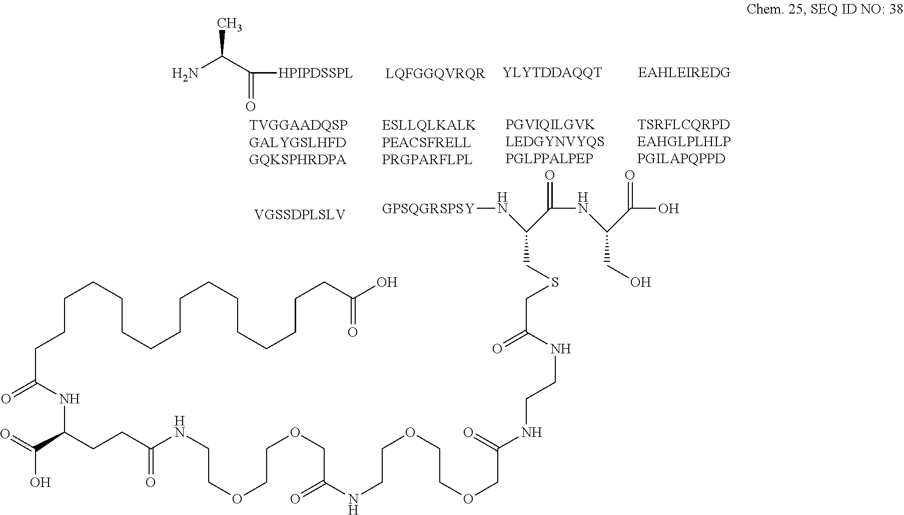

The compound was prepared as described in WO1016102562.

General Method for Measuring GLP-1 Activity To determine the GLP-1 receptor (GLP-1R) activity, or potency, of the GLP-1 moiety of the GLP-1/FGF21 fusion compounds and to assess how the activation of the receptor is potentially influenced by the presence of serum albumin, in vitro potency assays in cells expressing the human GLP-1 receptor were performed in the absence and presence of 1% (w/v) human serum albumin (HSA) as described below.

An increase in EC50 value (decrease in potency) in the presence of serum albumin indicates binding to serum albumin and represents a method to predict a protracted pharmacokinetic profile of the test substance in animal models.

The more potent the compound, the lower the EC50 value. A compound is considered a highly potent GLP-1 receptor agonist when its EC50 is below approximately 50 pM, such as 20 pM, wherein the EC50 is measured using an assay as described herein without the addition of HSA. A compound is considered to have medium potency when its EC50 value is 50-250 pM. A compound is considered to have a poorer potency when its EC50 value is 250-1000 pM. A compound is considered impotent when its EC50 is above 1000 pM.

### Assay Principle

Activation of GLP-1 receptors leads to increased cellular concentrations of cyclic AMP (cAMP). Consequently, transcription is activated by promotors containing multiple copies of the cAMP response element (CRE). It is thus possible to measure GLP-1 receptor activity using a CRE-luciferase reporter gene introduced into Baby Hamster Kidney (BHK) cells co-expressing the GLP-1 receptor.

### Cells and Assay Reagents

Cell stocks were prepared by culturing of a stably transfected cell line expressing the human GLP-1 receptor and the CRE responsive luciferase (CRE-Luc) reporter gene (BHK 467-12A KZ-10 prepared according to methods known to the person skilled in the art) in growth medium consisting of DMEM (Gibco, 61965-026) supplemented with 10% FBS (Gibco, 16140-071), 1% Pen/Strep (Gibco, 15140-122), 1 mM Na-Pyrovate (Gibco, 11360-039), 1 mg/mL G418 (Gibco, 10131-027) and 240 nM MTX (Pfizer, 15936). Cells at approximately 80-90% confluence were washed once in PBS and loosened from the cell flasks with Versene (Gibco, 15040-033). After centrifugation, the cell pellet was dissolved and diluted to 1.5×10E6 cells/mL in medium consisting of DMEM (Gibco, 61965-026) supplemented with 20% FBS (Gibco, 16140-071), 1% Pen/Strep (Gibco, 15140-122), 1 mM Na-Pyrovate (Gibco, 11360-039), 1 mg/mL G418 (Gibco, 10131-027), 240 nM MTX (Pfizer, 15936) and 10% DMSO (Sigma, D2650). Cells were aliquoted and stored at −180° C. until use.

The assay buffer consisted of DMEM without phenol red (Gibco, 11880-028) supplemented with 1× GlutaMAX (Gibco, 35050-038), 10 mM HEPES (Gibco, 15630-056), 1% (w/v) ovalbumin (Sigma, A5503) and 0.1% (v/v) Pluronic F-68 (Gibco, 24040-032).

### Procedure

To perform the assay, serial dilutions (10-fold dilutions, 8 concentrations pr. compound) of reference compounds and GLP-1/FGF21 fusion compounds were performed in assay buffer without HSA often starting from approximately 100-200 nM in a 96-well plate. Frozen stocks of human GLP-1R/CRE-Luc cells were thawed in a 37° C. water bath, washed once in PBS and diluted to 100.000 cells/mL in assay buffer with or without 2% (w/v) HSA (Sigma, A9511). For each dilution, 50 μL aliquots of reference compounds or GLP-1/FGF21 co-agonists were transferred to two 96-well assay plates (ThermoFisher, 237105) to which 50 μL of the cell suspension with or without 2% (w/v) HSA was added (5.000 cells/well). The assay plates were incubated for 3 hours at 37° C. in 5% CO₂, left at room temperature for 5 minutes after which 100 μL SteadyLite Plus (PerkinElmer, 6066759) was added to each well. Plates were sealed and incubated at room temperature with gentle shaking for 30 minutes while protected from light. Luminescence was detected on a luminescence plate reader e.g. a Synergy 2 (BioTek). The EC₅₀-values [pM] were calculated by non-linear curve fitting applying a four-parameter logistic model (Hill slope=1) using GraphPad Prism or by means of TIBCO Enterprise Runtime for R (TIBCO Software, Palo Alto, CA, USA).

### Example 2

The GLP-1 activity of the example compounds was investigated using the general method. The results are presented Table 6 (average of at least two separate concentration response curves). All Example compounds exhibited GLP-1 activity.

TABLE 6
GLP-1 potency of GLP-1/FGF21 fusion compounds.

| | GLP-1 receptor potency | GLP-1 receptor potency |
|---|---|---|
| Chem. no. | [EC50] pM 0% HSA | [EC50] pM 1% HSA |
| Chem. 24 | 5.6 | 169.8 |
| (comparator) | | |
| Chem. 9 | 67.4 | 396.6 |
| Chem. 10 | 13.8 | 78.7 |
| Chem. 11 | 9.8 | 66.6 |
| Chem. 12 | 9.5 | 102.8 |
| Chem. 13 | 54.6 | 393.8 |
| Chem. 14 | 18.0 | 390.7 |
| Chem. 15 | 223.5 | 4410.0 |
| Chem. 16 | 11.4 | 388.6 |
| Chem. 17 | 16.9 | 75.4 |
| Chem. 18 | 15.7 | 106.5 |
| Chem. 19 | 29.5 | 450.0 |
| Chem. 20 | 92.3 | 393.8 |
| Chem. 21 | 17.2 | 104.4 |
| Chem. 22 | 28.2 | 221.5 |
| Chem. 23 | 9.8 | 203.4 |

All fusion compounds showed activity on the GLP-1 receptor, which were comparable or higher in the presence of HSA. In vitro GLP-1R potency was dependent on the alkylation position. For both mono and double alkylated compounds the highest potency was found for alkylation at position 27C followed by position 36C, spacer C and 26C. In the presence of albumin, the same pattern was observed, however, the double alkylated compounds lost significantly more potency compared to mono alkylated compounds. For the mono alkylated 27C derivatives, a decrease in potency in the presence of albumin increased with increasing spacer length, although the longest spacer, 32xGAQP, in Chem. 23 had a potency comparable to Chem. 24 in the presence of albumin. 26R did not appear to impact the in vitro potency. As Chem. 24 has the protractor on position 26 it is surprising that in a fusion context the potency loss is more than 10 fold in the absence of HSA for the mono-alkylated compound and in the double-alkylated version a 40 fold loss in absence of HSA and 26 fold in the presence of HSA was observed.

### General Method for Investigating FGF21 Activity

The purpose of this example was to test the activation of FGF21 receptors of the Example fusion compounds. The in vitro FGF21 receptor potency was measured of FGF receptor activation in a whole cell assay.

The potencies of the GLP-1/FGF21 fusion compounds of Example 2 were determined in HEK (Human Embryonic Kidney cells) overexpressing human beta-klotho (BKL) as described further below.

In order to test the binding of the GLP-1/FGF21 fusion derivatives to albumin, the assay was performed in the absence of serum albumin as well as in the presence of human serum albumin (HSA) (0.1% final assay concentration). An increase in EC50 value (decrease in potency) in the presence of serum albumin for FGF21 derivatives would indicate binding to serum albumin and represents a method to predict a protracted pharmacokinetic profile of the test substance in animal models.

The results for GLP-1/FGF21 fusion compounds are shown in table 7. Chem. 25 is included for reference.

### Assay Principles

HEK293 cells endogenously express several FGF receptors, including FGFR1c, FGFR3c and FGFR4. These cells are unresponsive to FGF21 until transfected with the co-receptor beta-klotho (BKL). Activation of the FGF receptor/BKL complex leads to activation of the MAPK/ERK signalling pathway and phosphorylation of ERK. The level of phosphorylated ERK (pERK) at a given time point increases with increasing concentrations of FGF21. As described below the level of pERK was measured after 12 minutes of stimulation with a range of test compound concentrations.

### Assay Description

1) Day 1: The HEK293/beta-klotho cells were seeded with 50.000 cells/well in 96 well plates in DMEM w. 4500 mg/l glucose (Gibco #31966-021), supplemented with 10% HI FBS (Gibco #16140-71), 1% penicillin/streptomycin (Gibco #15140-122), 100 μg/ml Hygromycin B, (Gibco, #10687-010).
2) Day 2: 2 hours before addition of test compound the cell medium was exchanged with 100 μl basal medium (DMEM w. 4500 mg/l glucose (Gibco #31966-021)).
3) The test compounds were diluted in 2× assay medium (DMEM w. 4500 mg/l glucose (Gibco #31966-021) supplemented with 0.01% Tween20), warmed to 37° C., added to the cells with basal medium (100 μl) and incubated at 37° C. for 12 minutes. The test compounds were tested in the presence of and in the absence of 0.1% HSA (Sigma—A1887).
4) All medium was quickly removed and 50 μl lysis buffer was added pr. well. The plate was shaken for 10 minutes and the lysate was ready for measurement of pERK. 5) pERK is measured in 384 well plates with the AlphaScreen SureFire kit (PerkinElmer #TGRESB10K). This kit is based on ERK and pERK specific antibodies coupled to donor and acceptor beads. The presence of pERK will bring acceptor and donor beads in close proximity and a signal is generated that is read on EnVision plate reader.

The data were analysed using GraphPad Prism software. EC₅₀ values were calculated by the software, using non-linear regression, and reported in nM.

### Example 3

The FGF21 activity of the example compounds was investigated using the general method. The results are presented Table 7. (average of at least two independent experiments). All Example compounds exhibited FGF21 activity.

TABLE 7
FGF21 potency of GLP-1/FGF21 fusion
compounds in HEK293/BKL cells.

| | FGF21 receptor potency | FGF1 receptor potency |
|---|---|---|
| Chem. no | [EC50] nM 0% HSA | [EC50] nM 0.1% HSA |
| Met-FGF21 | 0.52 | 0.44 |
| (Comparator) | | |
| SEQ ID NO: 36 | | |
| Chem. 25 | 2.5 | 10.1 |
| (comparator) | | |
| Chem. 9 | 0.43 | 0.51 |
| Chem. 10 | 0.45 | 0.64 |
| Chem. 11 | 0.41 | 0.87 |
| Chem. 12 | 0.47 | 1.14 |
| Chem. 13 | 0.41 | 0.73 |
| Chem. 14 | 0.20 | 7.57 |
| Chem. 15 | 0.24 | 8.97 |
| Chem. 16 | 1.2 | 8.0 |
| Chem. 17 | 0.13 | 0.38 |
| Chem. 18 | 0.24 | 0.45 |
| Chem. 19 | 0.22 | 18.02 |
| Chem. 20 | 0.19 | 1.36 |
| Chem. 21 | 0.17 | 0.43 |
| Chem. 22 | 0.32 | 0.57 |
| Chem. 23 | 0.67 | 1.42 |

As can be seen from Table 7, all fusion compounds showed FGF21 receptor potency that was higher as compared to the FGF21 moiety alone (including the protractor substituent) In general, the FGF21 potency was improved compared to Chem. 25. In case of the mono-alkylated compounds the potency in presence of HSA only had a minor effect, which is most likely due to the absence of an albumin binder in the FGF21 part of the fusions. The double alkylated compounds had an improved potency compared to Chem. 25 in the absence of HSA, however, in the presence of HSA the potency was similar to Chem. 25 and presented a larger shift in the presence of albumin compared to Chem. 25.

### Example 4: General Method for Investigating Mean Residence Time (Pharmacokinetics)

The purpose of this study was to determine the mean residence time of the compounds in different species. Mice, minipigs and cynomolgus monkeys were selected to this end.

### Mean Residence Time (MRT)

MRT was decided to be used instead of T_{1/2} as initial fast decline in the plasma concentrations was observed, which could indicate a fast distribution phase and a very slow terminal elimination phase of the PK profile. In order not to emphasise too much on the slow terminal elimination phase, which would only represent few data point and a very low AUC and therefore most likely low efficacy, we decided to us the MRT that is a more robust parameter base on the total AUC of the PK profile involving all data of the profile. Results are shown in Table 8, 11 and 12.

### Example 4.1: Study in Mice

Dosing and sampling: Mice (male C57BL/6J (Mus musculus), Janvier Labs, Le Genest-Saint-Isle, France) age between 7 and 8 weeks of age were used in the study. The study used n=15 mice per test compound, using sparse sampling methodology with two or three blood samples from each mouse. n=3 per time-point. The animals were dosed intravenously at a dose of 5 nmol/kg of test compound (in buffer of 8 mM phosphate; 240 mM propylene glycol; 0.007% polysorbate 20; pH=8.2) at a concentration of 1 nmol/ml. Approximately 100 μl blood was sampled from the sublingual plexus of conscious mice according to the following regimen: at 5 min, 15 min, 30 min, 1 hour, 2 hours, 3 hours, 4 hours, 6 hours, 18 hours, 24 hours, 30 hours, 48 hours, and 72 hours after dosing. Blood was collected in a tube coated with EDTA (Sarstedt microvette 1293) and placed on ice until centrifuged at 4° C., 5 min, 6000G. Plasma (app. 50 μl) was transferred to 0.7 ml Micronic tubes and stored at −20° C. until analysis

### Bioassays:

Due to lack of an immunobased assay for detection of GLP-1/FGF21 compounds in plasma, using a combination of antibodies reacting to the GLP1 part and the FGF21 part, it was decided to use an assay to detect GLP1 and FGF21 separately. The chosen GLP1 assay was able to detect GLP1 with an intact N-terminus and the chosen FGF21 assay was able to detect the intact C-terminus of FGF21.

### FGF21 Assay

Sample analysis: Samples were analysed using Luminescence Oxygen Channeling Immunoassay (LOCI or AlphaLISA). Donor beads were coated with streptavidin (lot #2298681, PerkinElmer, USA) according to standard protocols, while acceptor beads were conjugated (lot #2356893, PerkinElmer, USA) with an in-house generated monoclonal antibody according to standard procedures (as exemplified by Lu et al (Journal of Biomedical Science (2020) 27:1)), specific for an epitope in the C-terminal of the FGF21 polypeptide of the test compound. The secondary antibody, a commercially available polyclonal antibody for human FGF21 (lot. BAF2539, R&D systems, USA), was biotinylated according to standard procedures. The three reactants were combined with the respective analyte and formed a two-sited immuno-complex. Illumination of the complex released singlet oxygen atoms from the donor beads. They were channeled into the acceptor beads and triggered a chemiluminescence response, which was measured in an EnVision plate reader (PerkinElmer, USA). The amount of light was proportional to the concentration of the test compound.

The protocol was as follows: 5 μL of plasma sample, and assay accuracy-controls (high, mid and low concentrations within the calibrator range) calibrators were applied to the appropriate wells of the white 384-well plates followed by a 15 μL mixture of acceptor beads coated with the in house monoclonal antibody described above (0.5 pg/well) and biotinylated PCA Human FGF21 (4.5 nM/well). The plates were incubated for 1 hour at room temperature. Then 30 μL streptavidin-coated donor-beads (2 pg/well) were added to each well and incubated for 30 min at room temperature. The plates were read in an Envision plate reader (Perkin Elmer) at room temperature using a filter having a bandwidth of 520-645 nm and excitation by a 680 nm laser. The total measurement time per well was 210 ms including a 70 ms excitation time.

Data analysis and reporting: Plasma concentration vs. time data after single IV dosing was subjected to a non-compartmental analysis in Phoenix™, WinNonlin®, (Pharsight®, St. Louis, Missouri, USA). The area under the plasma concentration-time profile was calculated by means of the log-linear trapezoidal rule. The area under the first moment curve was also calculated by the log-linear trapezoidal methods. The mean residence time was calculated as the ratio between the area under the first moment curve and the area under the plasma concentration-time curve. Terminal half-life was calculated using “individual best fit” of the log-linear regression of concentrations versus time and reported as harmonic mean and pseudo standard deviation.

### GLP-1 Assay

The assay and calculations were performed as described above for the FGF21 assay, using a biotin labelled inhouse produced monoclonal antibody against the N-terminus of GLP-1 together with an inhouse produced monoclonal antibody against the middle part of GLP-1 conjugated to acceptor beads.

TABLE 8
MRT obtained by GLP-1 assay and FGF21 assay.

| Chem. no. | GLP-1 MRT (h) | FGF21 intact MRT(h) |
|---|---|---|
| Chem. 9 | 2.8 | 2.6 |
| Chem. 10 | 2.5 | 2.9 |
| Chem. 11 | 3.2 | 3.2 |
| Chem. 12 | 6.05 | 5.59 |
| Chem. 13 | 2.9 | 2.7 |
| Chem. 14 | 13.3 | 19.0 |
| Chem. 16 | 15.3 | 19.6 |
| Chem. 17 | 2.9 | 2.6 |
| Chem. 18 | 4.0 | 3.6 |
| Chem. 19 | 17.0 | 21.1 |
| Chem. 20 | 5.11 | 4.1 |
| Chem. 21 | 2.5 | 2.4 |
| Chem. 22 | 3.8 | 3.2 |
| Chem. 23 | 9.8 | 9.4 |

The mean residence time found by the GLP-1 assay and the FGF21 assay were comparable and ranged from 2.5 h to 21 h (2.5 h to 17 h using the GLP-1 assay and 2.4 h to 21.1 h using the FGF21 assay). The MRT of the mono-alkylated compounds increased with increasing spacer length from 2.5 h (GLP-1 assay, Chem. 21) to 9.8 h (GLP-1 assay, Chem. 23). The double alkylated compounds showed significant longer MRT with compound Chem. 19 having the longest MRT of 17.0 h and 21.1 h using the GLP-1 and the FGF21 assay respectively.

### Example 4.2: Study in Mini Pigs

The overall aim is to verify that the compounds Chem. 16, Chem. 19 and Chem. 23 have a clearance and terminal half-life/MRT consistent with a once weekly dosing in humans.

### Dosing

The study was performed in eighteen (18) female Gottingen minipigs from Ellegaard Gottingen Minipigs A/S, Soro Landevej 302, DK-4261 Dalmose. At start of the acclimatisation period, the body weight of the pigs was approximately 15-20 kg and the age of 7-9 mounts. The animals were dosed i.v. or s.c. according to following schedule:

### Dosing Schedule

TABLE 9
Dosing schedule.

| | | Route of | Number | | Dosing | Number |
|---|---|---|---|---|---|---|
| | Chem. | adminis- | of | Dose | volume | of |
| Group | no. | tration | animals | (nmol/kg) | (ml/kg) | doses |
| 1 | Chem. | i.v. | 3 | 4 | 0.04 | 1 |
| 2 | 16 | s.c. | 3 | 4 | 0.04 | 1 |
| 3 | Chem. | i.v. | 3 | 4 | 0.04 | 1 |
| 4 | 19 | s.c. | 3 | 4 | 0.04 | 1 |
| 5 | Chem. | i.v. | 3 | 3.2 | 0.04 | 1 |
| 6 | 23 | s.c. | 3 | 3.2 | 0.04 | 1 |

i.v. administration: Intravenous (i.v.) injections were administered through ear-vein catheter. After the compound was dosed, the catheter was flushed with 10 mL of sterile saline
s.c. administration: The compound was dosed with a 1 mL or 2 mL syringe and a 25G × ⅝ (orange needle) needle in 5 mm depth (with a stopper on the needle) or a 21G × ¾ butterfly needle in 5 mm depth (with a stopper on the needle). The minipigs were s.c. dosed on the side of the neck. The needle was kept in the skin for approximately 10 sec. after dosing

### Blood Sampling

A full plasma concentration-time profile was obtained from each animal.

Blood samples were taken through the ear-vein catheter according to the schedule below.

Blood (1.3 ml) was collected in EDTA tubes (1.3 ml tube containing K3EDTA to yield 1.6 mg K3EDTA/ml blood (Sarstedt, Germany)). After each blood sample the catheter was flushed with 10 ml of sterile 0.9% NaCl and 10 IE/ml Heparin.

Samples were kept on wet ice for maximum 30 minutes until centrifugation (10 min, 4° C., 2000×g) and was transferred into Micronic tubes for measurement of exposure.

TABLE 10
Blood sampling schedule.

| Day 0 (dosing day) | Week 1 | Week 2 | Week 3 |
|---|---|---|---|
| Pre-dose | 24 hours | 10 days (240 h) | 17 days (408 h) |
| 5 min | 2 days (48 h) | 14 days (336 h) | 21 days (504 h) |
| 30 min | 4 days (96 h) | | |
| 1 h (60 min) | 7 days (168 h) | | |
| 2 h (120 min) | | | |
| 4 h (240 min) | | | |
| 8 h (480 min) | | | |

### Bioanalysis

The GLP-1/FGF21 fusion compounds were assayed in plasma from pigs by immunocapture, digestion by proteases and liquid chromatography mass spectrometry (LC-MS). The GLP-1/FGF21 fusion compounds were quantified using surrogate peptides covering the N- and C-terminal part of the compounds

In short, calibrators were prepared by spiking blank plasma with the relevant fusion compound in the range from 1 to 200 nM. 25 μL of calibrators, blank plasma or study samples were mixed with 25 μL of blank pig plasma (containing 50 nM internal standard), 195 ul PBS-buffer and 5.5 uL biotinylated monoclonal antibody (in-house produced, specific to the middle region of FGF21 (PGQKSPHRDPAPRGP)). The mixture was incubated at 37° C. for two hours. After incubation, 25 μL of Dynabeads MyOne Streptavidin T1 magnetic beads (10 mg/ml, ThermoFisher Scientific) were added and the mixture was incubated for one hour at room temperature. After incubation, the beads were washed three times with PBS-buffer and the GLP-1/FGF21 fusion compound was eluted from the beads by 100 uL elution buffer containing 10% acetonitrile, 1% formic acid and 0.005% Tween 20 in Milli-Q water. After elution, 50 μL of trypsin or LysC (0.02 ug/uL in 1 M TRIS-buffer (pH 9)) was added to the elution buffer. After 18 hours at 37° C., the digestion was terminated by adding 4.5 uL of formic acid. The mixture was centrifuged, and the supernatant was transferred to a microtiterplate (coated with BSA). The mixture was analysed by LC-MS using either an Accucore 150-C4 column from Thermo (100×2.1 mm ID; 2.6 m) operated at 60° C. or an Acquity UPLC Peptide BEH C18 from Waters (300A, 1.7 μm, 2.1*100 mm) operated at 80° C. A gradient elution was conducted with a Nexera UHPLC system (Shimadzu) using mobile phase A (consisting of milli-Q water with 0.1% formic acid and 5% acetonitrile) and mobile phase B (consisting of acetonitrile with 0.1% formic acid and 5% Milli-Q water). The flow was 0.6 ml/min. A TripleTOF 5600 mass spectrometer (Sciex) was used as detector and operated in positive electrospray ionisation mode. Calibration curves were used for calculating the concentration in the plasma samples. Quality control samples were included and the deviation between nominal and calculated concentration was below 20% (25% at the LLOQ).

### Pharmacokinetic Analysis

Plasma concentration-time data were analysed by non-compartmental pharmacokinetics using Phoenix 8 (Certara, Princeton, NJ, 08540 USA).

Calculations were performed using individual concentration-time values from each animals at each time point.

The following pharmacokinetic parameters were calculated at each occasion: AUC, AUC/Dose, AUC_{% Extrapol}, C₀, Cₘₐₓ, λ_{z}, tₘₐₓ, t_{1/2}, CL, CL/f, V_{z}, V_{z}/f, Vₛₛ, MRT and f.

### Treatment of Concentration Values Below the Lower Limit of Quantification (LLOQ)

Plasma concentrations below the LLOQ were treated as follows:

Pre-dose (0 h) plasma concentrations below the limit of detection appeared as “<LLOQ” in the data file. The values were automatically taken to be zero by Phoenix.

If one post dose concentration value was quantifiable and another value at the same dose and time was <LLOQ, the latter was set to ½LOD for calculation of mean concentrations. If the resulting mean value <LLOQ, this value was replaced by “<LLOQ” in the data file and consequently, it was not included in the calculations.

If one post dose (mean) concentration value was <LLOQ and the subsequent (mean) value was quantifiable, the former was set to ½LLOQ for calculation of pharmacokinetic parameters.

TABLE 11
Pharmacokinetic profiles of GLP-1 -
FGF21 fusion compounds in minipigs.

| | PK-LCMS | PK-LCMS | PK-LCMS | PK-LCMS |
|---|---|---|---|---|
| | MRT iv (h) | MRT iv (h) | MRT sc (h) | MRT sc (h) |
| Chem. no. | N-term | C-term | N-term | C-term |
| Chem. 23 | 92.4 | 90.1 | 151 | 108 |
| Chem. 16 | 116 | 117 | 149 | 156 |
| Chem. 19 | 109 | 109 | 134 | 167 |

The results show that after iv administration the same MRT (h) was obtained for the N-terminal peptide and the C-terminal peptide. After s.c. administration MRT for the N-terminal part of the mono-alkylated compound Chem. 23 was longer than MRT's for the C-terminal part. The opposite was the case in the double alkylated compound with one protractor in the spacer. In general the double alkylated compounds showed the longest MRT. All the compounds were evaluated to have MRT's in pigs long enough to support low dosing in humans.

### Example 4.3: Study in Cynomolgus Monkeys

Dosing and sampling: Non-naïve (but not previously dosed with FGF21 or GLP-1 products) female Vietnamese cynomolgus monkeys (Macaca fascicularis, Nafovanny/KHI Group, Vietman), aged at least 2 years at start of treatment and weights between 2.0-3.0 kg were used in the study. The study used n=3 monkeys per group. One group were dosed intravenously and one group were dosed subcutaneously at a dose of 5 nmol/kg of test compound (in aqueous solution comprising 10 mM phosphate; 2% (w/vol) glycol; pH=8.15) at a concentration of 1 mg/ml. Approximately 600 μl blood was sampled from the femoral vein according to the following regimen: Pre-dose, 5 min, 30 min, 1 hour, 2 hours, 4 hours, 8 hours, 24 hours, 48 hours, 96 hours, 168 hours, 240 hours, 336 hours, 408 hours and 504 hours post dosing. Blood was collected in a Teklab K3EDTA tubes containing 1.75 mg of EDTA/mL blood (part No 3K200PP) and placed on ice for a maximum of 30 min. until centrifuged at 2-8° C., 10 min, 2000 g. Plasma (at least 100 μl) was transferred to Micronic tubes and stored at −30-−10° C. until analysis.

The bioanalysis and pharmacokinetic analysis were performed as in the minipig study. Pharmacokinetic parameters are presented in Table 12.

TABLE 12
Pharmacokinetic profile of Chem. 23 in cynomolgus monkeys.

| | PK-LCMS | PK-LCMS | PK-LCMS | PK-LCMS |
|---|---|---|---|---|
| | MRT iv (h) | MRT iv (h) | MRT sc (h) | MRT sc (h) |
| Chem. no. | N-term | C-term | N-term | C-term |
| Chem. 23 | 52.9 | 44.9 | 63.2 | 56.0 |

A mean residence time of 56 h in monkeys was observed for the C-terminal part of compound Chem. 23 after s.c. dosing and was evaluated to be long enough to support ow dosing in humans.

### Example 5: Evaluation of the GLP-1 PD Effect of the Fusion Compounds

The purpose of this study was to investigate the GLP-1 effect of GLP-1/FGF21 fusion compounds by investigation their effect on acute food intake after single dose i.v administration in lean mice

FGF21 does not have an effect on food intake (FI) 24 hours after a single injection. Conversely, GLP-1 is known to reduce FI. Hence, a model of acute FI was used to investigate the pharmacodynamic effective dose and in vivo potency of the GLP-1 part of the GLP-1/FGF21 bifunctional molecules.

### Methods:

56 male C57BL6J mice age 8 weeks were used to measure acute FI in single housed mice in the BioDaq system (New Brunswick, NJ, USA) and kept in a reverse light/dark cycle (dark 11 am to 11 pm) with ad lib access to chow diet Altromin1324. The mice were allowed to acclimatize to the BioDaq cages for 14 days and then randomized on body weight into 7 groups of 8 mice/group. A baseline measurement of FI was collected for 24 hours prior to dosing. The mice were intravenously injected with 10 nmol/kg of Chem. 23 or the control compounds Chem. 24, Chem. 12, Chem. 14, Chem. 16, Chem. 19 or vehicle in the tail vein an hour before the onset of darkness. FI data was collected for 24 hours post dose. One animal in the Chem. 16 group was excluded due to technical issues.

Statistics was done in Graph Pad Prism 8.0.2 using Oneway ANOVA with Dunnett's correction, p<0.05. ***=p<0.001, ****=p<0.0001, ns=not significant

### Results

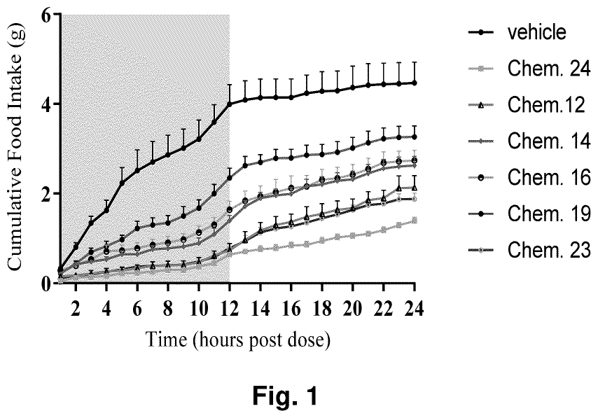

There were no significant differences in food intake between groups before administration of the compounds. After administration of the active compounds, food intake was significantly reduced in all groups compared to vehicle, with Chem. 23 reducing FI by 58%, followed by Chem. 12 (−52%), Chem. 14 (−41%), Chem. 16 (−39%), and Chem. 19 (−27%), with the positive control Chem. 24 displaying the largest reduction (−70%) over 24 hours FIG. 1.

All tested dual GLP-1/FGF21 compounds significantly reduced food intake with Chem. 23 and Chem. 12 displaying similar in vivo efficacy on the GLP-1 receptor as Chem. 24.

### Example 6: Evaluation of Effect on Body Weight of the Fusion Compounds

Evaluation of GLP-1/FGF21 fusion compounds in the low density lipoprotein receptor deficient mouse model (LDLr−/−) body weight lowering efficacy

The aim of following study was to evaluate the body weight (BW) on selected GLP−1/FGF21 fusion compounds in the LDLr−/− mouse model, a pro-atherosclerotic mouse model with severe dyslipidaemia and increased BW.

### Study Design

Eighty (80) male LDLr−/− mice (JAX, USA; STOCK:2207) were put on a high fat and cholesterol diet (WD; D12049B, Research Diets, USA) for at least 10 weeks before entering the study protocol. Two days prior to start of experiment, mice were randomized into eight groups based on their morning BW. The animals were dosed subcutaneously with test article or vehicle daily for 21 days at 12 PM. The dose of test article was adjusted daily based on BW. For group 3 (Chem. 24) the dose was up-titrated over five days starting with 1.0 ml/kg equal to 2 nmol/kg and ending up with 10 nmol/kg.

### Body Weight Data

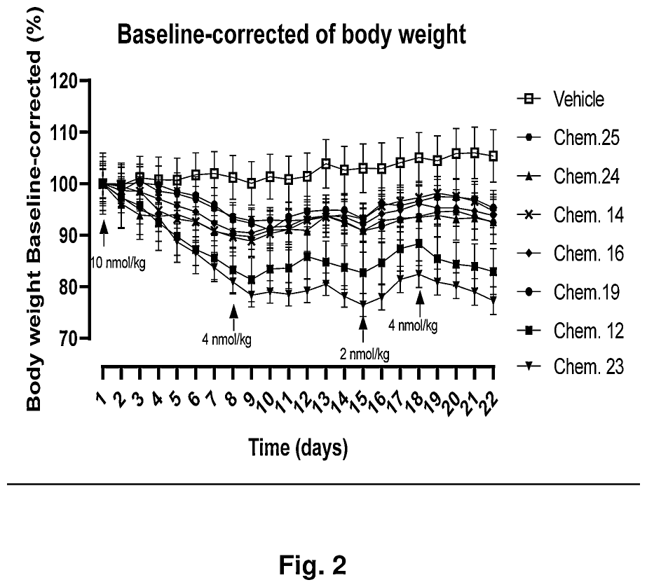

Daily baseline corrected body weight is shown in FIG. 2. All compounds showed a significant reduction in BW. The analogues Chem. 12 and Chem. 23 showed the most efficient BW lowering reaching to a reduction of more than 20% from baseline values after 7 days of dosing. This BW reduction by Chem. 12 and Chem. 23 was judged to be very powerful and due to ethical reasons the doses were lowered in all groups. The very efficient BW reduction continued for the Chem. 23 analogues thus the dose was further reduced to 2 nmol/kg. However, this dose was judged to be too low and the dose was once again adjusted to 4 nmol/kg which continued through out the study. Notably Chem. 25 (FGF21) and Chem. 24 showed similar significant BW reduction.

### Example 7: Chemical Stability: IsoAsp Formation

FGF21 has several aspartic acids, which are prone to isoAsp formation (D5, D24, D25, D38 and D102). A stability study was performed to evaluate the degree of isoAsp formation.

### Procedure Stability Study

Chem. 12, Chem. 14, Chem. 16, Chem. 19 and Chem. 23 (20 mg/ml) were prepared in 10 mM phosphate buffer, 2% glycerol, pH 8.2; filtered and aliquoted into dust free sterile HPLC vials and stored at quiescent temperatures (5C, 25, 37C), sampling every week for 4 weeks for chemical and physical stability

### Data Analysis by Peptide Mappinq

IsoAsp formation at the above mentioned positions was determined by peptide mapping and LC-MS. Samples were digested by trypsin (E:S 1:20 w/w; pH7.5; 2 hours@37° C., Barocycler: 72 cycles 90 sec/10 sec) and reduced (0.25 M DTT, pH7.5, 0.5 hour@37° C.) before LC-UV215-MS/MSMS analysis. The amount of isobaric tryptic peptides with IsoAsp (%) relative to the un-modified tryptic peptides was based on the areas using the extraction ion chromatogram (XIC). The following IsoAsp sites were identified directly in the isobaric tryptic peptides using electron-transfer dissociation (ETD) MS/MS i.e. 25IsoAsp, 38IsoAsp, and 102IsoAsp. No ETD reported ions was observed for the isobaric peptides with IsoAsp24 and IsoAsp25 (numbering of amino acid position according to SEQ ID NO: 2).

TABLE 13
102 isoAsp content at time zero and after 33 days at 37° C.

| Chem. no. | Tz (%) | 33 days at 37° C. (%) |
|---|---|---|
| Chem. 16 | 0.55 | 15.37 |
| Chem. 14 | 0.84 | 12.81 |
| Chem. 19 | 0.36 | 12.2 |
| Chem. 12 | 0.48 | 7.68 |
| Chem. 23 | 0.38 | 6.45 |
| Chem. 25 | 0.20* | 5.97 |

*21 days at 5° C.

### Results

IsoAsp formation was identified in position D5, D24, D25, D38 and D102 (numbering of amino acid position according to SEQ ID NO: 2) for all analogues analysed including the reference compound Chem. 25. Even though the isoAsp content was similar at time zero, the compounds containing two side chains (Chem. 14, Chem. 16 and Chem. 19) showed a higher level of all isoAsp derivatives than the compounds with one side chain (Chem. 12 and Chem. 23). The isoAsp formation at position 102 are shown in Table 13. 102 IsoAsp formation for compound Chem. 12 and Chem. 23 was on par with the isoAsp formation of the reference compound Chem. 25 after 33 days at 37° C.

1. A fusion protein comprising a polypeptide of Chem. 1: A-B-C,
wherein:
i. A is a GLP-1 polypeptide, which is an analogue of GLP-1 (7-37) (SEQ ID NO:1) with a maximum of 5 amino acid changes;
ii. B is a spacer consisting of 9 to 129 amino acids;
iii. C is an FGF21 polypeptide, which is an analogue of FGF21 (1-181) (SEQ ID NO: 2) with a maximum of 5 amino acid changes;
and one or more substituents, wherein the substituent is Chem 7: 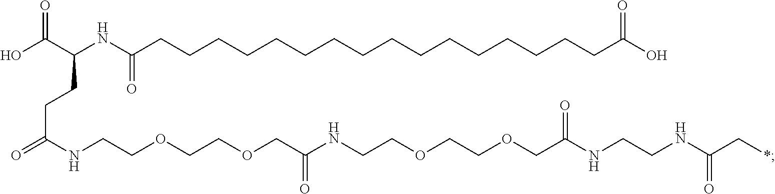
or a pharmaceutically acceptable salt thereof.

2. The fusion protein according to claim 1, wherein the spacer comprises at least two segments of GAQP.

3. The fusion protein according to claim 1, wherein B consists of SEQ ID NO: 13, SEQ ID NO:14, SEQ ID NO: 15, SEQ ID NO: 16, SEQ ID NO: 17 or SEQ ID NO: 18.

4. The fusion protein according to claim 1, wherein A comprises the following amino acid changes as compared to GLP-1 (7-37) (SEQ ID NO:1): 8G, 22E, 27C, 36G or 8G, 22E, 26R, 27C, 36G.

5. The fusion protein according to claim 1, wherein C comprises the following amino acid changes as compared to FGF21(1-181) (SEQ ID NO: 2): 121Q, 168L, 180C or 121Q, 168L, 171G, 180E.

6. The fusion compound according to claim 1 wherein the compound is Chem 16, Chem 19, or Chem. 23.

7. The fusion compound according to claim 1 having the structure of Chem. 23: 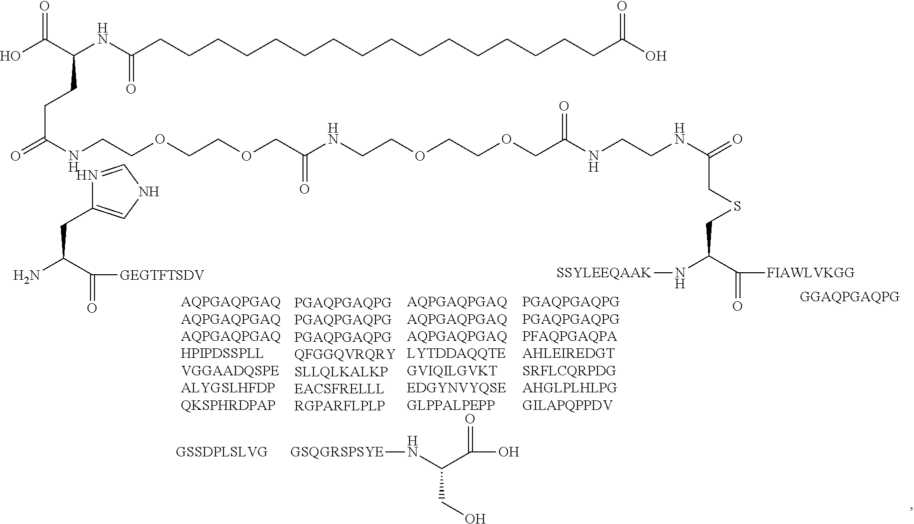
or
a pharmaceutically acceptable salt thereof.

8. The fusion compound according to claim 1 having the structure of Chem. 12: 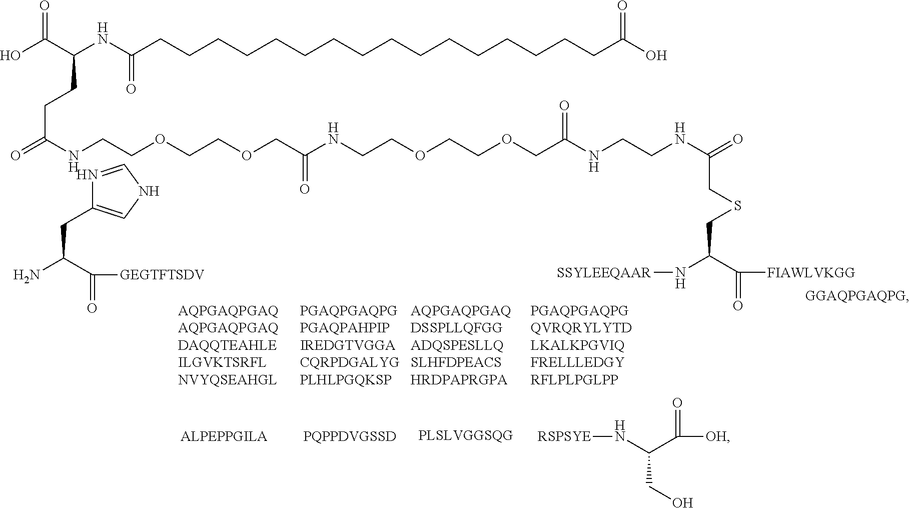
a pharmaceutically acceptable salt thereof.

9. The fusion compound according to claim 1 having the structure of Chem. 14: 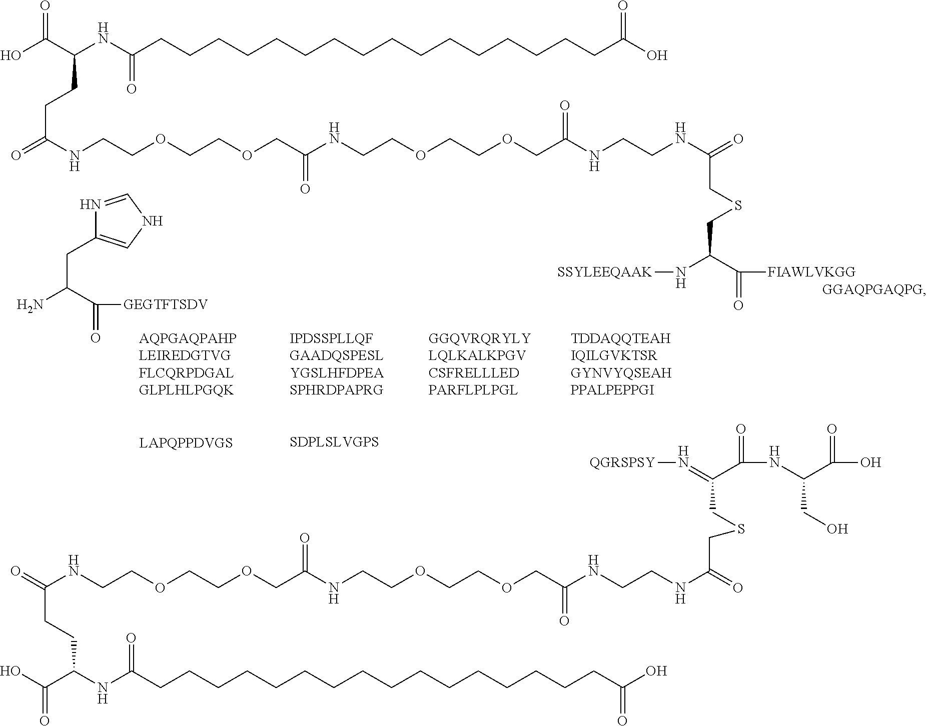
or
a pharmaceutically acceptable salt thereof.

10. The fusion compound according to claim 1 having the structure of Chem. 16: 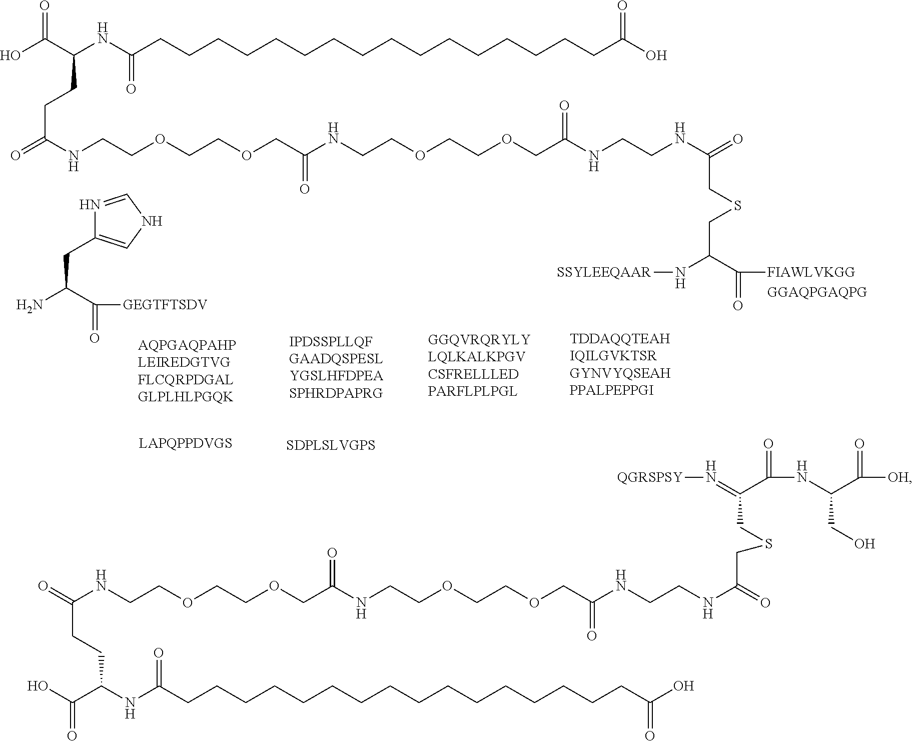
or a
pharmaceutically acceptable salt thereof.

11. The fusion compound according to claim 1 having the structure of Chem 19: 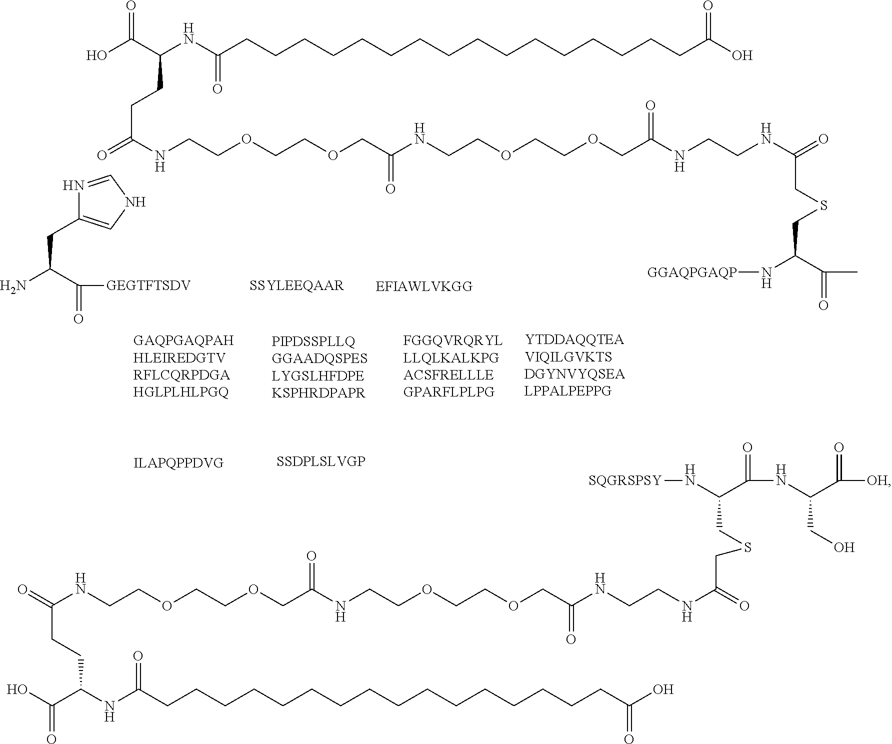
or
pharmaceutically acceptable salt thereof.

12. A pharmaceutical composition comprising a compound of claim 1, or a pharmaceutically acceptable salt thereof, with one or more pharmaceutically acceptable carriers, diluents, or excipients.

13. The pharmaceutical composition according to claim 12 with one or more additional pharmacologically active substances.

14. A method of treating obesity, diabetes mellitus, dyslipidemia, or the metabolic syndrome comprising administering a compound of claim 1, or a pharmaceutically acceptable salt thereof, to a patient in need thereof.

15. The method according to claim 14 wherein diabetes mellitus is Type 1 Diabetes.

16. A method of treating non-alcoholic fatty liver disease (NAFLD) comprising administering a compound of claim 1, or a pharmaceutically acceptable salt thereof, to a patient in need thereof.

17. The method according to claim 16 wherein NAFLD is non-alcoholic steatohepatitis (NASH).

18. A method of treating chronic kidney disease comprising administering a compound of claim 1, or a pharmaceutically acceptable salt thereof, to a patient in need thereof.
